(19) United States
(12) Patent Application Publication
(10) Pub. No.: US 2009/0202534 A1
(43) Pub. Date: Aug. 13, 2009

(54) COMPOSITIONS AND METHODS FOR MODULATING IMMUNE RESPONSES

(76) Inventors: Steven D. Levin, Seattle, WA (US); Frederick J. Ramsdell, Bainbridge Island, WA (US); Edward D. Howard, Seattle, WA (US); David W. Taft, Kirkland, WA (US); Mark W. Rixon, Issaquah, WA (US); LuAnne Hebb, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/260,335

(22) Filed: Oct. 29, 2008

Related U.S. Application Data
(60) Provisional application No. 60/983,664, filed on Oct. 30, 2007.

(51) Int. Cl. A61K 39/395; C07K 16/18; A61P 35/00

(52) U.S. Cl. 424/133.1; 530/387.9; 530/387.3; 424/139.1

(57) ABSTRACT

Disclosed is a newly identified CD28 family member that functions as lymphocyte inhibitory receptor termed pG6b, which is expressed on T cells. Methods and compositions for modulating pG6b-mediated negative signaling and interfering with the interaction of its counter-receptor for therapeutic, diagnostic, and research purposes are also disclosed.

### CROSS-REFERENCE TO RELATED APPLICATIONS

The present application claims the benefit of U.S. Patent Application Ser. No. 60/983,664, filed Oct. 30, 2007, which is incorporated by reference herein in its entirety.

### BACKGROUND OF THE INVENTION

Positive and negative costimulatory signals play critical roles in the modulation of T cell activity, and the molecules that mediate these signals have proven to be effective targets for immunomodulatory agents. Positive costimulation, in addition to T cell receptor (TcR) engagement, is required for optimal activation of naive T cells, whereas negative costimulation is believed to be required for the acquisition of immunologic tolerance to self, as well as the termination of effector T cell functions. Upon interaction with B7-1 or B7-2 on the surface of antigen-presenting cells (APC), CD28, the prototypic T cell costimulatory molecule, emits signals that promote T cell proliferation and differentiation in response to TcR engagement, while the CD28 homologue cytotoxic T lymphocyte antigen-4 (CTLA-4) mediates inhibition of T cell proliferation and effector functions (Chambers et al., Ann. Rev. Immunol, 19:565-594, 2001; Egen et al., Nature Immunol, 3:611-618, 2002).

Many autoimmune disorders are known to involve autoreactive T cells and autoantibodies. Agents that are capable of inhibiting or eliminating autoreactive lymphocytes without compromising the immune system's ability to defend against pathogens are highly desirable. Conversely, many cancer immunotherapies, such as adoptive immunotherapy, expand tumor-specific T cell populations and direct them to attack and kill tumor cells (Dudley et al., Science 298:850-854, 2002; Pardoll, Nature Biotech., 20:1207-1208, 2002; Egen et al., Nature Immunol, 3:611-618, 2002). Agents capable of augmenting tumor attack are highly desirable. In addition, immune responses to many different antigens (e.g., microbial antigens or tumor antigens), while detectable, are frequently of insufficient magnitude to afford protection against a disease process mediated by agents (e.g., infectious microorganisms or tumor cells) expressing those antigens. It is often desirable to administer to the subject, in conjunction with the antigen, an adjuvant that serves to enhance the immune response to the antigen in the subject. It is also desirable to inhibit normal immune responses to antigen under certain circumstances. For example, the suppression of normal immune responses in a patient receiving a transplant is desirable, and agents that exhibit such immunosuppressive activity are highly desirable.

Costimulatory signals, particularly positive costimulatory signals, also play a role in the modulation of B cell activity. For example, B cell activation and the survival of germinal center B cells require T cell-derived signals in addition to stimulation by antigen. CD40 counter-receptor present on the surface of helper T cells interacts with CD40 on the surface of B cells, and mediates many such T-cell dependent effects in B cells. Interestingly, negative costimulatory receptors analogous to CTLA-4 have not been identified on B cells. This suggests fundamental differences may exist in the way T cells and B cells are induced to respond to antigen, which has implications for mechanisms of self-tolerance as well as the inhibition of B cell effector functions, such as antibody production. Were a functional CTLA-like molecule to be found on B cells, the finding would dramatically shift our understanding of the mechanisms of B cell stimulation. Further, the identification of such receptors could provide for the development of novel therapeutic agents capable of modulating B cell activation and antibody production, and useful in the modulation of immunologic responses.

Accordingly, there is a need in the art for the identification of additional CD28 family members, and molecules derived therefrom, that have either or both a T cell costimulatory activity and/or a B cell costimulatory activity . This need is based largely on their fundamental biological importance and the therapeutic potential of agents capable of affecting their activity. Such agents capable of modulating costimulatory signals would find significant use in the modulation of immune responses, and are highly desirable.

The present invention provides such polypeptides for these and other uses that should be apparent to those skilled in the art from the teachings herein.

### BRIEF SUMMARY OF THE INVENTION

In one aspect, the present invention provides methods for modulating lymphocyte activity. Such methods generally include contacting a pG6b-positive lymphocyte with a bioactive agent capable of modulating pG6b-mediated signaling in an amount effective to modulate at least one lymphocyte activity. In certain embodiments, the lymphocyte is a T lymphocyte. Typical T lymphocyte activities that can be modulated in accordance with the present invention include, for example, activation, differentiation, proliferation, survival, cytolytic activity, and cytokine production. In a specific embodiment, the lymphocyte activity includes a host immune response to a target antigen (e.g., a pathogen antigen, a vaccine antigen, or a tumor-associated antigen other than a pG6b counter-receptor).

In some variations of the method, the agent includes an antagonist of pG6b-mediated signaling, such that contacting of the lymphocyte with the agent inhibits the attenuation of lymphocyte activity mediated by pG6b signaling. Typically, the antagonist includes a blocking agent capable of interfering with the functional interaction of pG6b and its counter-receptor. Particularly suitable blocking agents include, for example, anti-pG6b antibodies capable of specifically binding to the extracellular domain of a pG6b protein (e.g., to an amino acid sequence as set forth in SEQ ID NO:3 [extracellular domain of human pG6b] or SEQ ID NO:6 [extracellular domain of mouse pG6b]), and soluble pG6b fusion proteins. In some embodiments, contacting the lymphocyte with the antagonist of pG6b mediated signaling increases lymphocyte activity.

In other variations, the agent includes an agonist of pG6b-mediated signaling, such that contacting of the lymphocyte with the agent inhibits lymphocyte activity. Typically, the agonist comprises a mimicking agent capable of mimicking or augmenting the functional interaction of pG6b and its counter-receptor. Particularly suitable mimicking agents include, for example, anti-pG6b antibodies capable of specifically binding to the extracellular domain of a pG6b protein (e.g., to an amino acid sequence as set forth in SEQ ID NO:3 or SEQ ID NO:6), where binding of the antibody mimics or augments the functional interaction of pG6b and its counter-receptor.

In another aspect, a method for treating cancer in a patient is provided. Generally, the method for treating cancer includes administering to the patient an antagonist of pG6b-mediated signaling, where the administration is effective to increase a host immune response against tumor cells in the subject. In some embodiments, the tumor cells of the patient express a pG6b counter-receptor. Typically, the antagonist of pG6b-mediated signaling includes a blocking agent capable of interfering with the functional interaction of pG6b and its counter-receptor. Particularly suitable blocking agents include, for example, anti-pG6b antibodies capable of specifically binding to the extracellular domain of pG6b, such that binding interferes with the interaction of pG6b and its counter-receptor.

In yet another aspect, the present invention provides a method for treating a patient having an autoimmune disease characterized by the presence of autoreactive pG6b-positive lymphocytes. Generally, the method includes administering to the patient an agonist of pG6b-mediated signaling, where the administration is effective to inhibit an autoreactive immune response against non-lymphoid, non-tumor host cells expressing pG6b. Typically, the agonist includes a mimicking agent capable of mimicking or augmenting the functional interaction of pG6b and its counter-receptor. Particularly suitable mimicking agents include anti-pG6b antibodies capable of specifically binding to the extracellular domain of a pG6b protein (e.g., SEQ ID NO:3 or SEQ ID NO:6), where binding of the antibody mimics or augments the functional interaction of pG6b and its counter-receptor.

Also provided are isolated anti-pG6b antibodies. In some embodiments, an isolated anti-pG6b antibody of the invention is characterized in that (a) the antibody specifically binds to the extracellular domain of a pG6b protein (e.g., SEQ ID NO:3 or SEQ ID NO:6) and (b) the antibody, when covalently coupled to a microbead to yield an immobilized form the antibody, is capable of inhibiting TcR-mediated activation in a T cell in vitro, where the TcR-mediated activation includes contacting the T cell with an agonistic anti-CD3 antibody also coupled the microbead. In certain variations, the anti-pG6b antibody is further characterized in that the immobilized form of the antibody is capable of inhibiting the TcR-mediated activation by at least about 50% relative to a control T cell that is contacted with the anti-CD3 covalently coupled to a microbead in the absence of the anti-pG6b antibody. In yet other variations, the anti-pG6b antibody is further characterized in that the immobilized form of the antibody is capable of inhibiting TcR-mediated activation comprising contacting the T cell with the anti-CD3 antibody covalently coupled to the microbead and a soluble, agonistic anti-CD28 antibody. The anti-pG6b antibodies can be, e.g., polyclonal or monoclonal antibodies.

In some embodiments, an anti-pG6b antibody having one or more of the above characteristics is an isolated anti-pG6b monoclonal antibody that competes for binding to the extracellular domain of human pG6b (SEQ ID NO:3) with an antibody selected from the following: (a) the antibody produced by the hybridoma of clone designation number 337.1.4 (ATCC Patent Deposit Designation PTA-8730); (b) the antibody produced by the hybridoma of clone designation number 337.3.3 (ATCC Patent Deposit Designation PTA-8731); (c) the antibody produced by the hybridoma of clone designation number 337.6.5.1 (ATCC Patent Deposit Designation PTA-8728); and (d) the antibody produced by the hybridoma of clone designation number 337.8.35.3 (ATCC Patent Deposit Designation PTA-8729). In some such variations, the anti-pG6b antibody includes at least one, at least two, or at least three complementarity determining region(s) (CDRs) of the antibody selected from (a), (b), (c), and (d). For example, the anti-pG6b antibody can include a heavy chain variable region having CDRs H1, H2, and H3 of the antibody selected from (a), (b), (c), and (d); and/or can include a light chain variable region having CDRs L1, L2, and L3 of the antibody selected from (a), (b), (c), and (d). In more particular variations, the anti-pG6b antibody includes the heavy chain variable region, light chain variable region, or both the heavy and light chain variable regions, of the antibody selected from (a), (b), (c), and (d). In specific embodiments, the anti-pG6b antibody is the antibody selected from (a), (b), (c), and (d). In other embodiments, the anti-pG6b antibody is a humanized antibody comprising (i) a heavy chain variable region having CDRs H1, H2, and H3 of the antibody selected from (a), (b), (c), and (d); and (ii) a light chain variable region having CDRs L1, L2, and L3 of the antibody selected from (a), (b), (c), and (d). In some variations, the anti-pG6b antibody is a single-chain antibody such as, for example, a single-chain Fv (scFv).

In certain embodiments, an isolated anti-pG6b antibody of the invention is characterized in that (a) the antibody specifically binds to the extracellular domain of a pG6b protein (e.g., SEQ ID NO:3 or SEQ ID NO:6) and further has at least one of the following properties: (b) the antibody, in a soluble form, is capable of inhibiting TcR-mediated activation in a T cell in vitro, where the TcR-mediated activation includes contacting the T cell with a soluble, agonistic anti-CD3 antibody in the absence CD28-mediated co-stimulation; and (c) the antibody, in a soluble form, is capable of enhancing TcR-mediated activation in a T cell in vitro, where the TcR-mediated activation includes contacting the second T cell with the soluble anti-CD3 antibody and a soluble, agonistic anti-CD28 antibody. Typically, where the soluble form of the anti-pG6b antibody is capable of inhibiting TcR-mediated activation in an anti-CD3-stimulated T cell in vitro, the soluble antibody is capable of inhibiting the TcR-mediated activation by at least about 50% relative to a control T cell that is contacted with the soluble anti-CD3 antibody in the absence of CD28-mediated co-stimulation and in the absence of the anti-pG6b antibody. Where the soluble anti-pG6b antibody is capable of enhancing TcR-mediated activation in an anti-CD3/anti-CD28-stimulated T cell in vitro, where the TcR-mediated activation includes contacting the second T cell with the soluble anti-CD3 antibody and a soluble, agonistic anti-CD28 antibody, the soluble anti-pG6b antibody is typically capable of enhancing the TcR-mediated activation by at least about 20% relative to a control T cell that is contacted with both the soluble anti-CD3 antibody and the soluble anti-CD28 antibody in the absence of the anti-pG6b antibody. The anti-pG6b antibodies can be, e.g., polyclonal or monoclonal antibodies.

Such anti-pG6b antibodies are useful, for example, in various methods for modulating T cell activation. Accordingly, in still another aspect, methods for inhibiting or enhancing T cell activation using anti-pG6b antibodies as discussed above are provided. In some embodiments, a method for inhibiting TcR-mediated T cell activation includes contacting a T cell, in the absence of CD28-mediated T cell co-stimulation, with an effective amount of an antibody having the following properties: (a) the antibody specifically binds to the extracellular domain of a pG6b protein (e.g., SEQ ID NO:3 or SEQ ID NO:6) and (b) the antibody, in a soluble form is capable of inhibiting TcR-mediated activation in a T cell in vitro, where the TcR-mediated activation includes contacting the T cell with an agonistic anti-CD3 antibody in the absence CD28-mediated co-stimulation. Such methods can be performed, for example, in vitro, ex vivo, or in vivo. In some variations, the method is a method for treating an inflammatory or autoimmune condition in a subject; in some such variations, the anti-pG6b antibody is used under conditions in which CD28/B7 co-stimulatory pathways are suppressed, such as, e.g., in combination with a second agent having CD28/B7 inhibitory activity.

In other embodiments, a method for enhancing TcR-mediated T cell activation includes contacting a T cell, in the presence of CD28-mediated T cell co-stimulation, with an effective amount of an antibody having the following properties: (a) the antibody specifically binds to the extracellular domain of a pG6b protein (e.g., SEQ ID NO:3 or SEQ ID NO:6) and (b) the antibody, in a soluble form, is capable of enhancing TcR-mediated activation in a T cell in vitro, where the TcR-mediated activation includes contacting the T cell with the anti-CD3 antibody and an agonistic anti-CD28 antibody. Such methods can be performed, for example, in vitro, ex vivo, or in vivo. In some variations, the method is a method for treating cancer in a subject; in some such variations, the anti-pG6b antibody is in combination with a second agent having CD28/B7 stimulatory activity. Such variations are effective for increasing a subject's immune response against tumor cells, including, for example, tumor cells expressing a pG6b counter-receptor.

In yet other embodiments, a method for enhancing TcR-mediated T cell activation includes enhancing a T cell-mediated host immune response against a target antigen in a subject. Some such methods for enhancing a T cell-mediated host immune response against a target antigen in a subject include administering to the subject an effective amount of an antibody having the following properties: (i) the antibody specifically binds to the extracellular domain of a pG6b protein (e.g., SEQ ID NO:3 or SEQ ID NO:6) and (ii) the antibody, when covalently coupled to a microbead to yield an immobilized form the antibody, is capable of inhibiting TcR-mediated activation in a T cell in vitro, where the TcR-mediated activation includes contacting the T cell with an agonistic anti-CD3 antibody also coupled the microbead. Such methods can be performed, for example, in vitro, ex vivo, or in vivo. In preferred variations of such methods, the antibody is an isolated anti-pG6b monoclonal antibody that competes for binding to the extracellular domain of human pG6b (SEQ ID NO:3) with an antibody selected from the following: (a) the antibody produced by the hybridoma of clone designation number 337.1.4; (b) the antibody produced by the hybridoma of clone designation number 337.3.3; (c) the antibody produced by the hybridoma of clone designation number 337.6.5.1; and (d) the antibody produced by the hybridoma of clone designation number 337.8.35.3. Such antibodies can include any of the particular embodiments as described herein, including the monoclonal antibodies selected from (a), (b), (c), and (d), as well as those competitively binding antibodies comprising one or more CDRs from such a monoclonal antibody (e.g., humanized antibodies).

In some embodiments, enhancement of TcR-mediated T cell activation with an anti-pG6b antibody is used to treat cancer by increasing a host immune response against tumor cells in a subject. Some such methods for treating cancer in a patient include administering to the patient an antibody having the following properties: (i) the antibody specifically binds to the extracellular domain of pG6b and (ii) the antibody, when covalently coupled to a microbead to yield an immobilized form the antibody, is capable of inhibiting TcR-mediated activation in a T cell in vitro, where the TcR-mediated activation includes contacting the T cell with an agonistic anti-CD3 antibody also coupled the microbead. In certain variations, the anti-pG6b antibody is further characterized in that the immobilized form of the antibody is capable of inhibiting the TcR-mediated activation by at least about 50% relative to a control T cell that is contacted with the anti-CD3 covalently coupled to a microbead in the absence of the anti-pG6b antibody. In yet other variations, the anti-pG6b antibody is further characterized in that the immobilized form of the antibody is capable of inhibiting TcR-mediated activation comprising contacting the T cell with the anti-CD3 antibody covalently coupled to the microbead and a soluble, agonistic anti-CD28 antibody. In a specific variation, the anti-pG6b exhibits bead-coupled, in vitro activity against T cell activation similar or approximately equal to such in vitro T cell modulatory activity of a bead-coupled anti-CTLA-4 antibody. In certain preferred variations, the antibody is an isolated anti-pG6b monoclonal antibody that competes for binding to the extracellular domain of human pG6b (SEQ ID NO:3) with an antibody selected from the following: (a) the antibody produced by the hybridoma of clone designation number 337.1.4; (b) the antibody produced by the hybridoma of clone designation number 337.3.3; (c) the antibody produced by the hybridoma of clone designation number 337.6.5.1; and (d) the antibody produced by the hybridoma of clone designation number 337.8.35.3. Such antibodies can include any of the particular embodiments as described herein, including the monoclonal antibodies selected from (a), (b), (c), and (d), as well as those competitively binding antibodies comprising one or more CDRs from such a monoclonal antibody (e.g., humanized antibodies).

In another aspect, the present invention provides isolated cells that produce an antibody as described herein. In particular embodiments, the isolated cell is a hybridoma producing a monoclonal antibody. In specific variations, such a hybridoma is selected from the following hybridomas deposited with the American Type Tissue Culture Collection (ATCC; Manassas, Va.) patent depository: (a) the hybridoma of clone designation number 337.1.4 (ATCC Patent Deposit Designation PTA-8730); (b) the hybridoma of clone designation number 337.3.3 (ATCC Patent Deposit Designation PTA-8731); (c) the hybridoma of clone designation number 337.6.5.1 (ATCC Patent Deposit Designation PTA-8728); and (d) the hybridoma of clone designation number 337.8.35.3 (ATCC Patent Deposit Designation PTA-8729).

### BRIEF DESCRIPTION OF THE DRAWINGS

FIG. 1 depicts profiling of CD28 family member gene structure, including exon patterns and exon phasing. “S” denotes a first exon encoding a signal or leader sequence; “IgV” denotes a second exon encoding an IgV domain; and “TMD” denotes a third exon encoding a transmembrane domain. “C1” and “C2” denote, respectively, fourth and optional fifth exons encoding cytoplasmic domain(s). The numbers 0 and 2 denote the exon phasing between the exons.

FIGS. 2A-2D depict pG6b expression on pG6b-transfected P815 cells. Hybridoma pools positive for specific binding to pG6b-mFc in ELISA antibody were analyzed for ability to bind via FACS analysis to p815/pG6b cells (2A and 2C) but not parental p815 cells (2B and 2D) using a FITC-conjugated rat anti-mouse secondary antibody. Relative pG6b expression levels are shown on the y-axis as either mean fluorescence intensity (2A and 2B) or percentage of cells positive for anti-pG6b binding (2C and 2D).

FIGS. 3A-3D depict pG6b expression on resting CD4⁺ cells (3A and 3C) and CD8⁺ cells (3B and 3D) in human PBMCs derived from two different donors (Donor #1—3A and 3B; Donor # 2—3C and 3D).

FIG. 4 depicts increase in pG6b expression by T cell activation. Human PBMCs were stimulated with anti-CD3+ anti-CD28 monoclonal antibodies and analyzed for pG6b expression on CD4⁺ cells (4A) and CD8⁺ cells (4B) at 24, 48, and 72 hours by FACS with mAb anti-pG6b (337.8.35) or a control IgG mAb coupled to A647 dye. Untreated cells were similarly analyzed by FACS as a 0 time point.

FIGS. 5A and 5B depict increased expression of pG6b on CD4⁺ naïve cells (CD45RA⁺; FIG. 5B) relative to CD4⁺ memory cells (CD45RO⁺; FIG. 5A). Cells were analyzed by FACS with mAb anti-pG6b (337.8.35) coupled to A647 dye. CD4⁺ naïve cells were 37% positive for pG6b expression as compared to 23% of CD4⁺ memory cells.

FIGS. 6A and 6B depict effects of bead-coupled anti-pG6b antibodies on CD4⁺ T cells. Anti-pG6b antibodies were covalently coupled to tosylactivated 4.5μ beads together with anti-CD3 mAb. A control IgG1 were each also coupled to beads together with anti-CD3. T cells were cultured in the presence of antibody-coupled beads either in the absence (6A) or presence (6B) of soluble CD28 mAb and assessed for proliferation after 3 days on an LSRII (Becton Dickinson). Anti-pG6b mAbs (337.1, 337.2, 337.3, 337.4, 337.5, 337.6, 337.1.7, and 337.3.3) as well as anti-pG6b polyclonal antibody E9194 inhibited anti-CD3-induced proliferation (FIG. 6A). Anti-pG6b mAbs also inhibited anti-CD3/CD28-induced proliferation, while anti-pG6b polyclonal antibody E9194 slightly enhanced proliferation under these conditions (FIG. 6B).

FIGS. 7A and 7B depict effects of bead-coupled anti-pG6b antibodies on CD4⁺ (7A) and CD8⁺ (7B) T cells. Anti-pG6b mAbs 337.1.7 and 337.8.35.3 and pAb E9194 were each covalently coupled to tosylactivated 4.5μ beads together with anti-CD3 mAb. CTLA-4 mAb and a control IgG1 were each also coupled to beads together with anti-CD3. T cells from three different donors were cultured in the presence of antibody-coupled beads and assessed for proliferation after 3 days on an LSRII (Becton Dickinson). Anti-pG6b antibodies inhibited T cell proliferation relative to control mouse IgG1, with mAb 337.1 showing a greater effect than mAb 337.8.35.3 and pAb E9194 and an effect approximately equal to that observed for anti-CTLA4 mAb.

FIGS. 8A and 8B depict effects of bead-coupled anti-pG6b antibodies on CD4⁺ (8A) and CD8⁺ (8B) T cells. Anti-pG6b mAbs 337.1.7 and 337.8.35.3 and pAb E9194 were each covalently coupled to tosylactivated 4.5μ beads together with anti-CD3 mAb. CTLA-4 mAb and a control IgG1 were each also coupled to beads together with anti-CD3. T cells from three different donors were cultured in the presence of antibody-coupled beads and soluble anti-CD28 mAb and assessed for proliferation after 3 days on an LSRII (Becton Dickinson). Anti-pG6b mAbs 337.1.7 and 337.8.35.3 inhibited T cell proliferation relative to control mouse IgG1, with mAb 337.1 showing a slightly greater effect than mAb 337.8.35.3 and an effect approximately equal to that observed for anti-CTLA4 mAb. pAb E9194 enhanced T cell proliferation relative to control IgG1.

FIGS. 9A-9D depict effects of bead-coupled anti-pG6b antibodies on the expression of the early activation markers CD69 and CD25 on CD4⁺ (9A and 9B) and CD8⁺ (9C and 9D) T cells. T cells treated as described for FIGS. 7A and 7B above were analyzed for CD69 and CD25 expression by FACS. Anti-pG6b mAbs 337.1.7 and 337.8.35.3 inhibited anti-CD3-induced expression of CD25 (FIGS. 9A [CD4⁺] and 9C [CD8⁺]) and CD69 (FIGS. 9B [CD4⁺] and 9D [CD8⁺]) relative to control mouse IgG1, with mAb 337.1 showing a slightly greater effect in some donors than mAb 337.8.35.3 and an effect approximately equal to that observed for anti-CTLA4 mAb.

FIGS. 10A-10D depict effects of soluble anti-pG6b antibodies on the CD4⁺ (10A and 10B) and CD8⁺ (10C and 10D) T cells. T cells were separately treated with soluble anti-pG6b mAbs (337.1, 337.2, 337.3, 337.4, 337.5, 337.6, 337.7, 337.1.7, 337.3.3) and pAb E9194, together with soluble anti-CD3 mAb, either in the absence (10A and 10C) or presence (10B and 10D) of soluble anti-CD28 mAb. Proliferation was assessed after 3 days on an LSRII (Becton Dickinson). Anti-pG6b pAb E9194 inhibited the response of human CD4⁺ and CD8⁺ cells stimulated with soluble anti-CD3 (FIGS. 10A and 11B.) Inclusion of anti-CD28 to the cultures containing polyclonal anti-pG6b E9194 resulted in an increased proliferative response above that observed with anti-CD3 alone. (FIGS. 10B and 11B).

### DETAILED DESCRIPTION OF THE INVENTION

### 1. Overview

The present invention is generally directed to the identification and characterization of pG6b as a member of the CD28 family of lymphocytic receptors. Thus, the present invention provides a receptor newly identified as a member of the CD28 family that is expressed on T lymphocytes. The receptor of the present invention is denominated “pG6b,” and is distinct from CD28, CTLA-4, ICOS, PD-1, and BTLA. Methods and compositions for modulating pG6b-mediated lymphocyte signaling such as, e.g., modulating the natural interaction of pG6b and its counter-receptor are also provided, having multiple therapeutic applications for immunological tolerance, autoimmunity, immunosuppression, and immunotherapy including cancer immunotherapy.

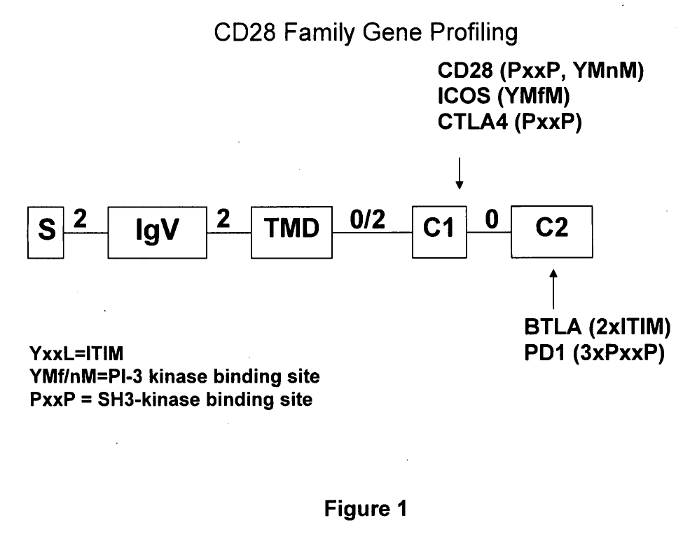

pG6b was identified by the present inventors as a CD28 family member based, at least in part, on CD28 family gene profiling. The present inventors appreciated that the CD28 family gene structure includes characteristic exon patterns, in which the first exon encodes a leader sequence, the second exon encodes an IgV domain, the third exon encodes a transmembrane domain, and that either one or two exons encode the cytoplasmic domain(s). (See FIG. 1.) Another characteristic feature of the CD28 family gene structure is the phasing of the exons, with a conserved phasing of 2 between exons 1 and 2 and between exons 2 and 3; a phasing of 0 or 2 between exons 3 and 4; and, wherein exon 5 is present, a conserved phasing of 0 between exons 4 and 5. (See id.)

As disclosed for the first time herein, pG6b is expressed on T cells and acts as a negative regulator of T lymphocyte activity, wherein signaling mediated by pG6b results in the inhibition of pG6b-positive lymphocyte activity. In pG6b-positive T cells pG6b signaling could, for instance, inhibit TcR-induced T cell responses, such as cell cycle progression, differentiation, survival, cytokine production and cytolytic activation. Further, in pG6b-positive B cells, pG6b signaling could inhibit B cell antigen receptor-induced B cell responses, such as cell cycle progression, differentiation, survival, antigen presentation and antibody production. These findings enable the use of therapeutic agents capable of interfering with or mimicking the interaction of pG6b and its counter-receptor to modulate lymphocyte activity for the purpose of treating, among other conditions, cancer and autoimmune diseases.

Accordingly, the present invention provides novel uses for pG6b modulators, such as pG6b agonists or antagonists. These modulators could be a soluble receptor or antibodies to pG6b or its receptor. The present invention also provides soluble pG6b polypeptide fragments and fusion proteins, for use in human inflammatory and autoimmune diseases. The pG6b antibodies and soluble pG6b receptors of the present invention can be used to modulate, agonize, block, increase, inhibit, reduce, antagonize or neutralize the activity of either pG6b or its counter-receptor(s) in the treatment of specific human diseases such as rheumatoid arthritis, psoriasis, psoriatic arthritis, arthritis, endotoxemia, inflammatory bowel disease (IBD), colitis, multiple sclerosis, and other inflammatory conditions disclosed herein.

An illustrative nucleotide sequence that encodes human pG6b (also interchangeably known as pG6bx1 is provided by SEQ ID NO:1; the encoded polypeptide is shown in SEQ ID NO:2. Analysis of a human cDNA clone encoding pG6b (SEQ ID NO:1) revealed an open reading frame encoding 242 amino acids (SEQ ID NO:2) comprising an extracellular domain of approximately 125 amino acid residues (residues 18-142 of SEQ ID NO:2; SEQ ID NO:3), a transmembrane domain of approximately 23 amino acid residues (residues 143-165 of SEQ ID NO:2), and an intracellular domain of approximately 76 amino acid residues (residues 166 to 241 of SEQ ID NO:2). pG6b also has an IgV domain of approximately 102 amino acid residues (residues 22-123 of SEQ ID NO:2). Within pG6b, there are two ITIM domains, LLYADL (amino acid residues 209-214 of SEQ ID NO:2) and TIYAVV (amino acid residues 235-240). The presence of an ITIM domain is an indication that pG6b can have an inhibitory effect. Within pG6b, there are also four SH-3-kinase binding domains, PPQP (amino acid residues 170-173 of SEQ ID NO:2), PIRP (amino acid residues 173-176 of SEQ ID NO:2), PQRP (amino acid residues 188-191 of SEQ ID NO:2) and PKIP (amino acid residues 199-200).

A variant soluble receptor is shown in SEQ ID NO:7.

An illustrative nucleotide sequence that encodes a murine pG6b is provided by SEQ ID NO:4; the encoded polypeptide is shown in SEQ ID NO:5. The extracellular domain is shown in SEQ ID NO:6.

Accordingly, in one aspect of the present invention, the present invention provides nucleic acid sequences encoding pG6b polypeptides, which are useful in the modulation of T lymphocyte activity and in the treatment of immune disorders, including autoimmune diseases, inflammation, psoriasis, IBD, ulcerative colitis and SLE.

The present invention also provides isolated polypeptides and epitopes comprising at least 15 contiguous amino acid residues of an amino acid sequence of SEQ ID NO:2 or 3. Illustrative polypeptides include polypeptides that either comprise, or consist of SEQ ID NO:3, an antigenic epitope thereof, or a functional pG6b binding fragment thereof. Moreover, the present invention also provides isolated polypeptides as disclosed above that agonize, bind to, block, inhibit, reduce, increase, antagonize or neutralize the activity of pG6b.

The present invention further provides antibodies and antibody fragments that specifically bind with such polypeptides. Exemplary antibodies include agonist antibodies, neutralizing antibodies, polyclonal antibodies, murine monoclonal antibodies, humanized antibodies derived from murine monoclonal antibodies, and human monoclonal antibodies. Illustrative antibody fragments include F(ab′)₂, F(ab)₂, Fab′, Fab, Fv, scFv, and minimal recognition units. Neutralizing antibodies preferably bind pG6b such that its interaction with its counter-receptor or counter-receptors is blocked, inhibited, reduced, antagonized or neutralized; anti-pG6b neutralizing antibodies such that such that its interaction with its counter-receptor or counter-receptors is blocked, inhibited, reduced, antagonized or neutralized are also encompassed by the present invention. The present invention further includes compositions comprising a carrier and a peptide, polypeptide, or antibody described herein.

Thus, in one embodiment, antagonists of pG6b signaling are provided for increasing T cell activation, and possibly B cell activation. In a preferred embodiment, such antagonists comprise blocking agents capable of interfering with the natural interaction of pG6b with its counter-receptor or counter-receptors, thereby inhibiting pG6b-mediated negative signaling and resulting in an increase in lymphocyte activation and proliferation and effector function.

In an alternative embodiment, agonists of pG6b signaling are provided for inhibiting T cell activation, and possibly B cell activation. In a preferred embodiment, such bioactive agents comprise mimicking agents capable of binding to pG6b and mimicking and/or augmenting the natural interaction of pG6b with its counter-receptor or counter-receptors, thereby resulting in inhibition of T cell activation (and possibly B cell) and proliferation and effector function.

In one embodiment, bioactive agents and methods for increasing and/or up-regulating B and T cell activity are provided. In a preferred embodiment, such bioactive agents comprise antagonists of pG6b-mediated signaling. In a particularly preferred embodiment, such bioactive agents comprise blocking agents as described herein, and in a specific embodiment, such blocking agents are capable of interfering with the interaction of pG6b and its counter-receptor. In a further embodiment, adjuvant compositions are provided utilizing pG6b blocking agents and other antagonists of pG6b-mediated signaling.

In an alternative embodiment, bioactive agents and methods for inhibiting and/or down-regulating B and T cell activity are provided. In a preferred embodiment, such bioactive agents comprise agonists of pG6b-mediated signaling. In a particularly preferred embodiment, such bioactive agents comprise mimicking agents as described herein, and in a specific embodiment, such mimicking agents are capable of replacing and/or augmenting the interaction of pG6b and its counter-receptor. In a further embodiment, immunosuppressive compositions are provided utilizing pG6b mimicking agents and other agonists of pG6b-mediated signaling.

In a further embodiment, methods and compositions for modulating immunoglobulin production by B cells is provided.

The methods and compositions described herein will find advantageous use in immunotherapy, including, e.g., autoimmunity, immune suppression, cancer immunotherapy and immune adjuvants.

In addition, the present invention also provides pharmaceutical compositions comprising a pharmaceutically acceptable carrier and at least one of such an expression vector or recombinant virus comprising such expression vectors. The present invention further includes pharmaceutical compositions, comprising a pharmaceutically acceptable carrier and a polypeptide or antibody described herein.

The present invention also contemplates anti-idiotype antibodies, or anti-idiotype antibody fragments, that specifically bind an antibody or antibody fragment that specifically binds a polypeptide comprising the amino acid sequence of SEQ ID NO:2 or 3 or a fragment thereof. An exemplary anti-idiotype antibody binds with an antibody that specifically binds a polypeptide consisting of SEQ ID NO:3.

The present invention also provides fusion proteins, comprising a pG6b polypeptide and an immunoglobulin moiety. In such fusion proteins, the immunoglobulin moiety may be an immunoglobulin heavy chain constant region, such as a human F_{c} fragment. The present invention further includes isolated nucleic acid molecules that encode such fusion proteins.

The present invention also provides polyclonal and monoclonal antibodies that bind to polypeptides comprising a pG6b extracellular domain such as monomeric, homodimeric, heterodimeric and multimeric receptors, including soluble receptors.

In another aspect, methods for modulating lymphocyte activity are provided comprising contacting a B and/or T lymphocyte with a bioactive agent capable of modulating pG6b activity. In one embodiment, the bioactive agent comprises an antagonist of pG6b activity such as, e.g., a pG6b or a pG6b counter-receptor blocking agent, resulting in an upregulation or increase in lymphocyte activity by preventing negative pG6b-mediated signaling. In an alternative embodiment, the bioactive agent comprises an agonist of pG6b activity such as, e.g., a pG6b or a pG6b counter-receptor mimicking agent, resulting in down-regulation of lymphocyte activity by replacing or augmenting pG6b-mediated negative signaling.

In a further aspect, methods for modulating lymphocyte activity are provided comprising contacting a B and/or T lymphocyte with a bioactive agent capable of modulating the interaction of pG6b with a pG6b counter-receptor. In one embodiment, a bioactive agent capable of interfering with the natural interaction of pG6b and a pG6b counter-receptor is employed to increase lymphocyte activity and proliferation such as, e.g., a pG6b counter-receptor or a pG6b blocking agent. In an alternative embodiment, a bioactive agent capable augmenting or replacing the natural interaction of pG6b and a pG6b counter-receptor is employed to inhibit lymphocyte activity and proliferation such as, e.g., a pG6b counter-receptor or pG6b mimicking agent.

Suitable pG6b blocking agents may be selected from the group comprising or consisting of soluble pG6b polypeptides and fusion proteins, anti-pG6b antibodies capable of binding to at least a portion of the extracellular domain of pG6b and interfering with pG6b-mediated signaling, small molecule inhibitors of pG6b receptor interaction with its ligands, and the like. Alternative pG6b antagonists further include antisense oligonucleotides directed to the pG6b nucleic acid sequence, inhibitory RNA sequences, small molecule inhibitors of pG6b expression and/or intracellular signaling, and the like.

Similarly, suitable pG6b counter-receptor blocking agents may be selected from the group comprising or consisting of anti-pG6b-counter-receptor antibodies capable of binding to at least a portion of the extracellular domain of a pG6b counter-receptor and interfering with the interaction of a pG6b counter-receptor and pG6b, small molecule inhibitors of the interaction between a pG6b counter-receptor and pG6b, soluble pG6b counter-receptor polypeptides and fusion proteins having modified pG6b counter-receptor amino acid sequences so as to interfere with the interaction of a pG6b counter-receptor and pG6b and incapable of activating pG6b-mediated signaling, and the like. Alternative pG6b counter-receptor antagonists include antisense oligonucleotides directed to a pG6b counter-receptor nucleic acid sequence, inhibitory RNA molecules, small molecule inhibitors of a pG6b counter-receptor expression, and the like.

Suitable pG6b mimicking agents may be selected from the group comprising or consisting of function-activating anti-pG6b antibodies capable of binding to at least a portion of the extracellular domain of pG6b and stimulating pG6b-mediated signaling, gene therapy vectors capable of recombinantly producing functional pG6b molecules intracellularly, small molecule enhancers of pG6b expression and/or pG6b-mediated signaling, and the like. Similarly, suitable pG6b counter-receptor mimicking agents may be selected from the group comprising or consisting of soluble pG6b counter-receptor polypeptides and fusion proteins capable of activating pG6b-mediated signaling, small molecule enhancers of the interaction between a pG6b counter-receptor and pG6b as well as enhancers of a pG6b counter-receptor expression, gene therapy vectors capable of recombinantly producing functional a pG6b counter-receptor molecules intracellularly, and the like.

Thus, in a more specific embodiment methods for stimulating, augmenting and/or increasing lymphocyte activity are provided comprising contacting a B or T lymphocyte with an antagonist of pG6b-mediated signaling, said antagonist comprising at least one bioactive agent selected from the group consisting of soluble pG6b polypeptides, soluble pG6b fusion proteins, anti-pG6b antibodies capable of binding to at least a portion of the extracellular domain of pG6b and interfering with pG6b-mediated signaling, small molecule inhibitors of pG6b expression and/or pG6b-mediated signaling, anti-pG6b counter-receptor antibodies capable of binding to at least a portion of the extracellular domain of a pG6b counter-receptor and interfering with the interaction of a pG6b counter-receptor and pG6b, small molecule inhibitors of the interaction between a pG6b counter-receptor and pG6b, soluble pG6b counter-receptor polypeptides and pG6b counter-receptor fusion proteins incapable of activating pG6b-mediated signaling, and interfering RNA sequences.

In a particularly preferred embodiment, methods for increasing a host immune response to antigenic stimulation are provided, comprising the administration to the host of at least one of the aforementioned antagonists of pG6b-mediated signaling. Desirably, the antigenic stimulation may be from pathogen antigens, vaccine antigens and/or tumor antigens.

In a specific embodiment, methods for stimulating a cellular immune response against tumor antigens other than a pG6b counter-receptor are provided, comprising administering to a cancer patient at least one of the subject antagonists or blocking agents to inhibit pG6b-mediated negative signaling and thereby increase the T cell response directed against tumor antigens other than a pG6b counter-receptor present in the cancerous tissue.

In a further specific embodiment methods for inhibiting, attenuating and/or decreasing lymphocyte activity are provided comprising contacting a B or T lymphocyte with an agonist of pG6b-mediated signaling, said agonist selected from the group consisting of soluble pG6b counter-receptor polypeptides and pG6b counter-receptor fusion proteins capable of activating pG6b-mediated signaling, function-activating anti-pG6b antibodies capable of binding to at least a portion of the extracellular domain of pG6b and stimulating pG6b-mediated signaling, gene therapy vectors capable of recombinantly producing functional pG6b molecules intracellularly, small molecule enhancers of pG6b expression and/or pG6b-mediated signaling, small molecule enhancers of the interaction between a pG6b counter-receptor and pG6b, small molecule enhancers of pG6b counter-receptor expression, and gene therapy vectors capable of recombinantly producing functional pG6b counter-receptor molecules intracellularly.

In a particularly preferred embodiment, methods for suppressing a host immune response to antigenic stimulation are provided, comprising the administration to the host of at least one of the aforementioned agonists of pG6b-mediated signaling. Desirably, the antigenic stimulation may be from self antigens in the context of autoimmune disease, or from donor antigens present in transplanted organs and tissues.

In an alternative aspect, the present invention provides bioactive agents and methods for modulating the interaction of a pG6b-counter-receptor-expressing cell and a pG6b-expressing lymphocyte. In a preferred embodiment, bioactive agents and methods for interfering with the interaction of a pG6b-counter-receptor-positive tumor cells with T cells are provided, resulting in inhibition of negative pG6b-mediated signaling. In an especially preferred embodiment, the T cell is a CD4⁺ cell or a CD8⁺ cell. In a further embodiment, the CD4⁺ T cell is a Th1 cell.

In another preferred embodiment, bioactive agents and methods for mimicking or enhancing the interaction of a pG6b-counter-receptor-positive, non-tumor, non-lymphoid cells with pG6b-positive T cells are provided, thereby decreasing T cell activity. In an especially preferred embodiment, the T cell is a CD4⁺ T cell or a CD8⁺ T cell. In a further embodiment, the CD4⁺ T cell is a Th1 cell.

In a further aspect, methods for treating cancers characterized by the presence of a pG6b-counter-receptor-expressing tumor cells are provided. In one embodiment, these methods comprise administering to a mammalian subject at least one of the antagonists of pG6b-mediated signaling disclosed herein, either alone or in conjunction with alternative cancer immunotherapy, chemotherapy and/or radiotherapy protocols. In a preferred embodiment, at least one pG6b or pG6b counter-receptor blocking agent is administered to a subject having pG6b-counter-receptor-positive tumor cells, wherein said blocking agent is capable of interfering with the interaction of pG6b and a pG6b counter-receptor and inhibiting pG6b-mediated signaling. Preferably, administration of said blocking agents is effective to increase T cell activity directed against tumor antigens other than a pG6b counter-receptor on the tumor cells, and in particular, to increase cytotoxic T cell activity. Still more preferably, administration of the subject antagonists is effective to inhibit the growth of the pG6b-counter-receptor-expressing tumor cells.

It is also contemplated that the subject pG6b and/or a pG6b counter-receptor blockade provided herein will find synergistic combination with CTLA-4 blockade as described in U.S. Pat. Nos. 5,855,887; 5,811,097; and 6,051,227, and International Publication WO 00/32231, the disclosures of which are expressly incorporated herein by reference.

In a further aspect, methods for treating autoimmune disorders characterized by the absent or aberrant expression of a pG6b counter-receptor in non-tumor non-lymphoid host cells subjected to autoimmune attack are provided. In one embodiment, these methods comprise administering to a mammalian subject at least one of the agonists of pG6b-mediated signaling disclosed herein, either alone or in conjunction with alternative immunotherapy and/or immunosuppressive protocols. In a preferred embodiment, at least one pG6b or a pG6b counter-receptor mimicking agent is administered to a subject having autoreactive pG6b-positive lymphocytes, wherein said mimicking agent is capable of replacing and/or augmenting the interaction of pG6b and a pG6b counter-receptor and replacing or increasing pG6b-mediated signaling. Preferably, administration of said mimicking agents is effective to decrease autoreactive lymphocyte activity directed against non-tumor non-lymphoid host cells, and particularly autoreactive CD8⁺ CTL and CD4⁺ Th1 activity, and B cell activity.

In a still further aspect, methods for improving the outcome of organ and tissue transplantation and prolonging graft survival are provided. In one embodiment, these methods comprise administering to a transplant recipient at least one of the agonists of pG6b-mediated signaling disclosed herein, either alone or in conjunction with alternative immunotherapy and/or immunosuppressive protocols. In a preferred embodiment, at least one pG6b or pG6b counter-receptor mimicking agent is administered to the transplant recipient, wherein said mimicking agent is capable of replacing and/or augmenting the interaction of pG6b and a pG6b counter-receptor and replacing or increasing pG6b-mediated signaling. Preferably, administration of said mimicking agents is effective to decrease the recipient immune response against donor antigens present in the graft, particularly the cytolytic CTL response and the B cell response. Still more preferably, administration of the subject mimicking agents is effective to bias to T helper cell response from an unfavorable Th-1 type response to a more favorable Th-2 type response, as described in more detail herein.

The present invention also provides compositions and methods for inhibiting autoimmune responses. In a preferred embodiment, compositions and methods for inhibiting the activity of autoreactive T and B cells that specifically recognize autoantigens are provided. Desirably, these compositions and methods may be used to inhibit killing of non-tumor cells mediated by one or more autoantigens.

Preferred compositions for use in the treatment of autoimmune disease comprise the agonists of pG6b-mediated signaling described herein including, e.g., the above-described mimicking agents. Especially preferred agents include pG6b protein fragments comprising the pG6b extracellular domain (SEQ ID NO:3), or a portion thereof, pG6b-Ig fusion proteins comprising the pG6b extracellular domain (SEQ ID NO:3), or a portion thereof; function-activating anti-pG6b antibody; peptides that mimic pG6b or its counter-receptor (mimetics); and small molecule chemical compositions that mimic the natural interaction of pG6b with its counter-receptor. Also preferred are compositions capable of binding to pG6b, either in a cross-linking fashion or as polyclonal mixtures.

Also contemplated in the present invention are genetic approaches to autoimmune disease. Particularly, gene therapy may be used to increase the level of pG6b expression on T cells, and/or increase the level of expression of its counter-receptor on non-lymphoid cells that are subject to attack by autoreactive lymphocytes. The use of isoforms or variants of pG6b that exhibit elevated specific activity is also contemplated, the object of each method being to potentiate signaling that is suppressive to T cell activation.

The present invention also provides compositions and methods for treating cancer, and in particular, for increasing the activity of pG6b-positive T lymphocytes against tumor cells. In some embodiments, the method is for increasing activity of pG6b-positive T lymphocytes against tumor cells expressing a pG6b counter-receptor. Desirably, these compositions and methods may be used to inhibit the growth of tumor cells, such as, for example, tumor cells capable of expressing a pG6b counter-receptor.

Preferred compositions for use in the treatment of cancer are the antagonists of pG6b-mediated signaling described herein including, e.g., pG6b blocking agents. Especially preferred agents include anti-pG6b antibodies; protein fragments comprising the pG6b extracellular domain, or a portion thereof; pG6b-Ig fusion proteins comprising the pG6b extracellular domain, or a portion thereof; function-blocking anti-pG6b antibody; peptides that mimic pG6b (mimetics); and small molecule chemical compositions that interfere with the natural interaction of pG6b and its counter-receptor.

Also contemplated in the present invention are genetic approaches to the treatment of cancer. Particularly, gene therapy may be used to decrease the level of pG6b expression on T cells, and/or decrease the level of expression of pG6b or its counter-receptor on tumor cells. The use of isoforms of pG6b that exhibit dominant negative activity is also contemplated, the object of each method being to inhibit signaling that is normally suppressive to T cell activation. Genetic approaches may involve the use of tissue and cell specific promoters to target expression of pG6b dominant negative variants, antisense nucleic acids, or small inhibitory RNAs to T cells and tumor cells, respectively. The methods may additionally involve the use of tumor-targeted viruses, or other delivery vehicles that specifically recognize tumor cells. The methods may additionally involve the use of T cell-targeted viruses, or other delivery vehicles that specifically recognize T cells.

Particularly preferred are agents that may be selectively targeted to tumor cells, and effect a decrease in pG6b expression in tumor cells without reducing the level of pG6b expression in non-tumor cells to deleterious levels. Highly preferred are agents that have a precursor form. These “prodrugs” are converted to their active form in the vicinity of tumor tissue typically by an enzymatic activity that is restricted in its distribution to the vicinity of the tumor.

Also highly preferred are agents that can be combined with targeting moieties that selectively deliver the agent to a tumor. These targeting moieties provide a high local concentration of the agent in the vicinity of the tumor tissue, and reduce the amount of agent that must be administered to effect the desired response.

Also contemplated in the present invention is the use of combination therapy to treat cancer, as described above.

In a preferred embodiment, immunization is done to promote a tumor-specific T cell immune response. In this embodiment, a bioactive agent that inhibits pG6b activation is administered in combination with a tumor-associated antigen. The combination of a tumor-associated antigen and a pG6b-inhibitory/counter-receptor functional-mimetic promotes a tumor specific T cell response, in which T cells encounter a lower level of inhibition than exerted by the tumor tissue in the absence of the bioactive agent.

In one aspect, the present invention provides a medicament for the treatment of cancer.

The present invention also provides compositions and methods for modulating normal but undesired immune responses involving T and B cell activity. In a preferred embodiment, compositions and methods for inhibiting the host lymphocyte response to transplanted tissue and organs are provided. Desirably, these compositions and methods may be used to prolong the survival of grafted tissue. Preferred compositions for use in the prevention of acute and/or chronic graft rejection comprise the agonists of pG6b-mediated signaling described herein including, e.g., the above-described mimicking agents. Especially preferred agents include pG6b polypeptides comprising the pG6b extracellular domain (SEQ ID NO:3), or a portion thereof, pG6b-Ig fusion proteins comprising the pG6b extracellular domain (SEQ ID NO:3), or a portion thereof, function-activating anti-PG6B antibodies; peptides that mimic Its counter-receptor (mimetics); and small molecule chemical compositions that mimic the natural interaction of pG6b and its counter-receptor. In addition to their utility in general immunosuppressive strategies, the subject agonists of pG6b-mediated signaling described herein may also have important implications for tolerance induction in tissue and organ transplantation, by biasing the recipient T helper cell immune response away from an unfavorable Th-1-type response and towards a more favorable Th-2 type response.

In one aspect, the present invention provides a medicament for use in transplantation and immune suppression.

Also provided are adjuvant compositions comprising at least one of the above-described pG6b and/or a pG6b counter-receptor blocking agents as well as other antagonists of pG6b-mediated signaling. Also provided are immunosuppressant compositions comprising at least one of the above-described pG6b and/or a pG6b counter-receptor mimicking agents as well as other agonists of pG6b-mediated signaling.

It is further contemplated that the subject compositions and methods may be synergistically combined with immunotherapies based on modulation of other T cell costimulatory pathways, and with CD28, ICOS, PD-1, CTLA-4 and/or BTLA modulation in particular.

In an alternative aspect, the present invention provides methods of screening for bioactive agents that are useful for modulating T cell activation. Bioactive agents identified by the screening methods provided herein may be used to react with a pG6b counter-receptor-expressing cells or pG6b-expressing cells in order to interfere with the interaction between pG6b-expressing B and/or T cells and pG6b-counter-receptor-expressing non-lymphoid cells, and thereby antagonize the function of the pG6b/pG6b counter-receptor interaction. Alternatively, bioactive agents may be used to react with a pG6b counter-receptor-expressing cells or pG6b-expressing cells in order to mimic a pG6b counter-receptor/pG6b interaction, effecting T cell inhibition in the absence of the pG6b/pG6b counter-receptor interaction. Alternatively, bioactive agents may be used to modify the natural pG6b/pG6b counter-receptor interaction in some way, for example, to increase the association and augment the inhibitory signal.

In an alternative aspect, the invention provides expression vectors comprising the isolated pG6b and/or a pG6b counter-receptor nucleic acid sequences disclosed herein, recombinant host cells comprising the recombinant nucleic acid molecules disclosed herein, and methods for producing pG6b and/or pG6b counter-receptor polypeptides comprising culturing the host cells and optionally isolating the polypeptide produced thereby.

In a further aspect, transgenic non-human mammals are provided comprising a nucleic acid encoding a pG6b and/or a pG6b counter-receptor protein as disclosed herein. The pG6b or pG6b counter-receptor nucleotides are introduced into the animal in a manner that allows for increased expression of levels of a pG6b or a pG6b counter-receptor polypeptide, which may include increased circulating levels. Alternatively, the pG6b or pG6b counter-receptor nucleic acid fragments may be used to target endogenous pG6b or pG6b counter-receptor alleles in order to prevent expression of endogenous pG6b or pG6b counter-receptor nucleic acids (i.e., generates a transgenic animal possessing a pG6b or a pG6b counter-receptor protein gene knockout). The transgenic animal is preferably a mammal, and more preferably a rodent, such as a rat or a mouse.

These and other aspects of the invention will become evident upon reference to the following detailed description. In addition, various references are identified below and are incorporated by reference in their entirety.

### P 2. Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1.47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

A “cloning vector” is a nucleic acid molecule, such as a plasmid, cosmid, or bacteriophage that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces pG6b from an expression vector. In contrast, pG6b can be produced by a cell that is a “natural source” of pG6b, and that lacks an expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes. For example, a fusion protein can comprise at least part of a pG6 b polypeptide fused with a polypeptide that binds an affinity matrix. Such a fusion protein provides a means to isolate large quantities of pG6busing affinity chromatography.

The term “receptor” denotes a cell-associated protein that binds to a bioactive molecule termed a “counter-receptor.” This interaction mediates the effect of the counter-receptor on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular counter-receptor-binding domain and an intracellular effector domain that is typically involved in signal transduction. In certain membrane-bound receptors, the extracellular counter-receptor-binding domain and the intracellular effector domain are located in separate polypeptides that comprise the complete functional receptor.

In general, the binding of counter-receptor to receptor results in a conformational change in the receptor that causes an interaction between the effector domain and other molecule(s) in the cell, which in turn leads to an alteration in the metabolism of the cell. Metabolic events that are often linked to receptor-counter-receptor interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

A “soluble receptor” is a receptor polypeptide that is not bound to a cell membrane. Soluble receptors are most commonly counter-receptor-binding receptor polypeptides that lack transmembrane and cytoplasmic domains, and other linkage to the cell membrane such as via glycophosphoinositol (gpi). Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Soluble receptors can be monomeric, homodimeric, heterodimeric, or multimeric, with multimeric receptors generally not comprising more than 9 subunits, preferably not comprising more than 6 subunits, and most preferably not comprising more than 3 subunits. Receptor polypeptides are said to be substantially free of transmembrane and intracellular polypeptide segments when they lack sufficient portions of these segments to provide membrane anchoring or signal transduction, respectively. Soluble receptors of class I and class II cytokine receptors generally comprise the extracellular cytokine binding domain free of a transmembrane domain and intracellular domain. For example, representative soluble receptors for pG6b include, for instance the soluble receptor as shown in SEQ ID NO:3. It is well within the level of one of skill in the art to delineate what sequences of a known B7 family member comprise the extracellular domain free of a transmembrane domain and intracellular domain. Moreover, one of skill in the art using the genetic code can readily determine polynucleotides that encode such soluble receptor polypeptides.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, such as 96%, 97%, or 98% or more pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

As used herein, the term “immunomodulator” includes cytokines, stem cell growth factors, lymphotoxins, co-stimulatory molecules, hematopoietic factors, and the like, and synthetic analogs of these molecules.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/counter-receptor pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

The term “antibody,” as used herein, refers to immunoglobulin polypeptides and immunologically active portions of immunoglobulin polypeptides, i.e., polypeptides of the immunoglobulin family, or fragments thereof, that contain an antigen binding site that immunospecifically binds to a specific antigen (e.g., the extracellular domain of pG6b). Thus, as used herein, the term “antibody” refers to an intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies, ScFv. “Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide-linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al., J. Mol. Biol. 186:651, 1985; Novotny and Haber, Proc. Natl. Acad. Sci. U.S.A. 82:4592, 1985).

An immunoglobulin light or heavy chain variable region consists of a “framework” region interrupted by three hypervariable regions. Thus, the term “hypervariable region” refers to the amino acid residues of an antibody which are responsible for antigen binding. The hypervariable region comprises amino acid residues from a “Complementarity Determining Region” or “CDR” (i.e., residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest (Public Health Service, National Institutes of Health, Bethesda, Md., 5th ed. 1991) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol. 196: 901-917, 1987) “Framework Region” or “FR” residues are those variable domain residues other than the hypervariable region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen.

The term “genetically altered antibodies” means antibodies wherein the amino acid sequence has been varied from that of a native antibody. Because of the relevance of recombinant DNA techniques in the generation of antibodies, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with cells and other effector functions. Changes in the variable region will be made in order to improve the antigen binding characteristics.

In addition to intact antibodies (such as native antibodies produced by the body in response to the presence of an antigen, or engineered variants thereof that substantially preserve the full native structure), immunoglobulins may exist in a variety of other forms including, for example, Fv or single-chain Fv, Fab, and (Fab′)₂, as well as diabodies, linear antibodies, multivalent or multispecific hybrid antibodies (see, e.g., Lanzavecchia et al., Eur. J. Immunol. 17, 105, 1987) and in single chains (see, e.g., Huston et al., Proc. Natl. Acad. Sci. U.S.A., 85, 5879-5883, 1988; and Bird et al, Science, 242, 423-426, 1988. (See also, generally, Hood et al., “Immunology” (Benjamin, N.Y., 2nd ed. 1984); and Hunkapiller and Hood, Nature, 323, 15-16, 1986).

Accordingly, in certain variations, an antibody is an “antibody fragment,” which is a portion of an intact antibody comprising the antigen binding site and capable of binding to its antigen. As previously indicated, an antibody fragment can include, e.g., F(ab′)₂, F(ab)₂, Fab′, Fab, and the like. A “Fab fragment” comprises one light chain and the C_{H1} and variable regions of one heavy chain. The heavy chain of a Fab molecule cannot form a disulfide bond with another heavy chain molecule. A “Fab′ fragment” contains one light chain and one heavy chain that contains the variable region and more of the constant region, between the C_{H1} and C_{H2} domains, such that an interchain disulfide bond can be formed between two heavy chains to form a F(ab′)₂ molecule. A “F(ab′)₂ fragment” contains two light chains and two heavy chains containing the variable region and a portion of the constant region between the C_{H1} and C_{H2} domains, such that an interchain disulfide bond is formed between two heavy chains. Regardless of structure, an antibody fragment binds with the same antigen that is recognized by the intact antibody. For example, an anti-pG6b monoclonal antibody fragment binds to an epitope of pG6b.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448, 1993.

The term “chimeric antibody” or “chimeric antibodies” refers to antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant domain from a human antibody, although other mammalian species may be used. Specifically, a chimeric antibody is produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody.

As used herein, the term “human antibody” includes an antibody that has an amino acid sequence of a human immunoglobulin and includes antibodies isolated from human immunoglobulin libraries or from animals transgenic for one or more human immunoglobulin genes and that do not express endogenous immunoglobulins, as described, for example, by Kucherlapati et al in U.S. Pat. No. 5,939,598.

Accordingly, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. Mutations in the frameworks may be required to preserve binding affinity when an antibody is humanized. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

An “anti-idiotype antibody” is an antibody that binds with the variable region domain of an immunoglobulin. In the present context, an anti-idiotype antibody binds with the variable region of an anti-pG6b antibody, and thus, an anti-idiotype antibody mimics an epitope of pG6b.

As used herein, a “therapeutic agent” is a molecule or atom which is conjugated to an antibody moiety to produce a conjugate which is useful for therapy. Examples of therapeutic agents include drugs, toxins, immunomodulators, chelators, boron compounds, photoactive agents or dyes, and radioisotopes.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See generally Ford et al., Protein Expression and Purification 2:95 (1991). DNA molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

The term “monoclonal antibody” refers to an antibody that is derived from a single cell clone, including any eukaryotic or prokaryotic cell clone, or a phage clone, and not the method by which it is produced. Thus, the term “monoclonal antibody” as used herein is not limited to antibodies produced through hybridoma technology.

As used herein, the term “antibody component” includes both an entire antibody and an antibody fragment.

An “immunoconjugate” is a conjugate of an antibody component with a therapeutic agent or a detectable label.

As used herein, the term “antibody fusion protein” refers to a recombinant molecule that comprises an antibody component and a pG6b polypeptide component. Examples of an antibody fusion protein include a protein that comprises a pG6b extracellular domain, and either an F_{c} domain or an antigen-binding region.

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

An “anti-sense oligonucleotide specific for pG6b” or a “pG6b anti-sense oligonucleotide” is an oligonucleotide having a sequence (a) capable of forming a stable triplex with a portion of the pG6b gene, or (b) capable of forming a stable duplex with a portion of an mRNA transcript of the pG6b gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

The term “variant pG6b gene” refers to nucleic acid molecules that encode a polypeptide having an amino acid sequence that is a modification of SEQ ID NO:2. Such variants include naturally-occurring polymorphisms of pG6b genes, as well as synthetic genes that contain conservative amino acid substitutions of the amino acid sequence of SEQ ID NO:2. Additional variant forms of pG6b genes are nucleic acid molecules that contain insertions or deletions of the nucleotide sequences described herein. A variant pG6b gene can be identified, for example, by determining whether the gene hybridizes with a nucleic acid molecule having the nucleotide sequence of SEQ ID NO: 1, or its complement, under stringent conditions.

Alternatively, variant pG6b genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology: Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

Regardless of the particular method used to identify a variant pG6b gene or variant pG6b polypeptide, a variant gene or polypeptide encoded by a variant gene may be functionally characterized the ability to bind specifically to an anti-pG6b antibody. A variant pG6b gene or variant pG6b polypeptide may also be functionally characterized the ability to bind to its counter-receptor or counter-receptors, using a biological or biochemical assay described herein.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, 0-globin, and myoglobin are paralogs of each other.

As used herein, the term “immune response” includes both T and/or B cell responses, i.e., cellular and/or humoral immune responses. In one embodiment, the compositions and methods disclosed herein can be used to reduce or enhance helper T cell (Th) responses, and more preferably, Th1 cell responses. In another embodiment, the compositions and methods disclosed herein can be used to reduce or, enhance cytotoxic T cell (Tc) responses. The claimed methods can be used to reduce or enhance both primary and secondary immune responses and effector function (e.g., cytolytic activity, cytokine and antibody production, and antigen presentation). The immune response of a subject can be readily determined by the skilled artisan using methods well known in the art, for example, by assaying for antibody production, immune cell proliferation, the release of cytokines, the expression of cell surface markers, cytotoxicity, etc.

By “pG6b signaling,” “pG6b-mediated signaling,” “pG6b-mediated negative signaling” and variations thereof is meant intracellular signaling in lymphocytes caused by the binding and/or activation of the pG6b receptor by its corresponding ligand(s) resulting in attenuation and/or down-regulation of lymphocyte activity. In one aspect, pG6b-mediated signaling comprises activation of SHP-1 and/or SHP-2.

“Lymphocyte activity” as used herein refers to the immunological processes of B and T cell activation, proliferation, differentiation and survival, as well as associated effector immune functions in lymphocytic cells including cytolytic activity (Tc cells), cytokine production (Th cells), antibody production (B cells), and antigen presentation (B cells). As noted above, there are numerous assays well-known to the skilled artisan for detecting and/or monitoring such processes, including but not limited to the assays described in the examples provided herein.

As used herein, the phrase “interaction of pG6b and its counter-receptor” refers to direct physical interaction (e.g. binding) and/or other indirect interaction of a functional pG6b counter-receptor molecule with a functional pG6b receptor on a lymphocyte, resulting in stimulation of the pG6b receptor and associated intracellular pG6b signaling. Similarly, the phrase “natural interaction of pG6b and its counter-receptor” refers to direct physical interaction (e.g. binding) and/or other indirect interaction of a functional and endogenously expressed counter-receptor molecule with a functional and endogenously expressed pG6b receptor on a lymphocyte, resulting in stimulation of the pG6b receptor and associated intracellular pG6b signaling.

As used herein, the term “blocking agent” includes those agents that interfere with the interaction of pG6b and its counter-receptor, and/or that interfere with the ability of the counter-receptor to inhibit lymphocyte activity, e.g., as measured by cytokine production and/or proliferation. The term “blocking agent” further includes agents that inhibit the ability of pG6b to bind a natural ligand, and/or that interfere with the ability of pG6b to inhibit T cell activity. Exemplary agents include function-blocking antibodies, as well as peptides that block the binding pG6b with its counter-receptor but which fail to stimulate pG6b-mediated signaling in a lymphocyte (e.g., pG6b fusion proteins), peptidomimetics, small molecules, and the like. Preferred blocking agents include agents capable of inhibiting the inducible association of pG6b with SHP-1 and/or SHP-2, or the signal transduction that derives from the interaction of SHP-1 and/or SHP-2 with pG6b.

As used herein, the term “mimicking agent” includes those agents that mimic the interaction of pG6b and its counter-receptor, and/or that augment, enhance or increase the ability of pG6b and/or its counter-receptor to inhibit lymphocyte activity. Exemplary agents include function-activating antibodies, as well as peptides that augment or enhance the ability of pG6b to bind with its counter-receptor or substitute for the counter-receptor's role in stimulating pG6b-mediated signaling (e.g., Its counter-receptor fusion proteins), peptidomimetics, small molecules, and the like.

The term “inhibit” or “inhibition of” as used herein means to reduce by a measurable amount, or to prevent entirely.

The term “microbead” or “bead” as used herein refers to a solid-phase particle that measures between about 1 μm and about 10 μm along any given axis, typically substantially spherical particles having a diameter between about 1 μm and about 10 μm, or typically between about 1 μm and about 6 μm (e.g., 4.5 μm or 5 μm). Examples of microbeads include latex and paramagnetic beads such as those typically used in immunological assays (e.g., tosylactived beads).

The present invention includes functional fragments of pG6b genes. Within the context of this invention, a “functional fragment” of a pG6b gene refers to a nucleic acid molecule that encodes a portion of a pG6b polypeptide which is a domain described herein or at least specifically binds with an anti-pG6b antibody.

Due to the imprecision of standard analytical methods, molecular weights and lengths of polymers are understood to be approximate values. When such a value is expressed as “about” X or “approximately” X, the stated value of X will be understood to be accurate to 10%.

### 3. Production of pG6b Polynucleotides or Genes

Nucleic acid molecules encoding a human pG6b gene can be obtained by screening a human cDNA or genomic library using polynucleotide probes based upon SEQ ID NO: 1. These techniques are standard and well-established, and may be accomplished using cloning kits available by commercial suppliers. See, for example, Ausubel et al. (eds.), Short Protocols in Molecular Biology, 3^{rd} Edition, John Wiley & Sons 1995; Wu et al., Methods in Gene Biotechnology, CRC Press, Inc. 1997; Aviv and Leder, Proc. Nat'l Acad. Sci. USA 69:1408 (1972); Huynh et al., “Constructing and Screening cDNA Libraries in λgt10 and λgt11,” in DNA Cloning: A Practical Approach Vol. I, Glover (ed.), page 49 (IRL Press, 1985); Wu (1997) at pages 47-52.

Nucleic acid molecules that encode a human pG6b gene can also be obtained using the polymerase chain reaction (PCR) with oligonucleotide primers having nucleotide sequences that are based upon the nucleotide sequences of the pG6b gene or cDNA. General methods for screening libraries with PCR are provided by, for example, Yu et al., “Use of the Polymerase Chain Reaction to Screen Phage Libraries,” in Methods in Molecular Biology, Vol 15: PCR Protocols: Current Methods and Applications, White (ed.), Humana Press, Inc., 1993. Moreover, techniques for using PCR to isolate related genes are described by, for example, Preston, “Use of Degenerate Oligonucleotide Primers and the Polymerase Chain Reaction to Clone Gene Family Members,” in Methods in Molecular Biology, Vol 15: PCR Protocols: Current Methods and Applications, White (ed.), Humana Press, Inc. 1993. As an alternative, a pG6b gene can be obtained by synthesizing nucleic acid molecules using mutually priming long oligonucleotides and the nucleotide sequences described herein (see, for example, Ausubel (1995)). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol 15: PCR Protocols: Current Methods and Applications, White (ed.), pages 263-268, (Humana Press, Inc. 1993), and Holowachuk et al., PCR Methods Appl. 4:299 (1995)). For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

### 4. Production of pG6b Gene Variants

The present invention provides a variety of nucleic acid molecules, including DNA and RNA molecules that encode the pG6b polypeptides disclosed herein. Those skilled in the art will readily recognize that, in view of the degeneracy of the genetic code, considerable sequence variation is possible among these polynucleotide molecules. Moreover, the present invention also provides isolated soluble monomeric, homodimeric, heterodimeric and multimeric receptor polypeptides that comprise at least one pG6b receptor subunit that is substantially homologous to the receptor polypeptide of SEQ ID NO:2. Thus, the present invention contemplates pG6b polypeptide-encoding nucleic acid molecules comprising degenerate nucleotides of SEQ ID NO:1, and their RNA equivalents.

Table 1 sets forth the one-letter codes to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons, encompassing all possible codons for a given amino acid, are set forth in Table 2.

TABLE 2

| Amino | One Letter | | Degenerate |
|---|---|---|---|
| Acid | Code | Codons | Codon |
| Cys | C | TGC TGT | TGY |
| Ser | S | AGC AGT TCA TCC | WSN |
| | | TCG TCT | |
| Thr | T | ACA ACC ACG ACT | CAN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC | MGN |
| | | CGG CGT | |
| Lys | K | AAA AAG | AAR |
| Met | M | ATG | ATG |
| Ile | I | ATA ATC ATT | ATH |
| Leu | L | CTA CTC CTG CTT | YTN |
| | | TTA TTG | |
| Val | V | GTA GTC GTG GTT | GTN |
| Phe | F | TTC TTT | TTY |
| Tyr | Y | TAC TAT | TAY |
| Trp | W | TGG | TGG |
| Ter | · | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding an amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences of SEQ ID NO:2. Variant sequences can be readily tested for functionality as described herein.

Different species can exhibit “preferential codon usage.” In general, see, Grantham et al, Nucl. Acids Res. 8:1893 (1980), Haas et al. Curr. Biol. 6:315 (1996), Wain-Hobson et al., Gene 13:355 (1981), Grosjean and Fiers, Gene 18:199 (1982), Holm, Nuc. Acids Res. 14:3075 (1986), Ikemura, J. Mol. Biol. 158:573 (1982), Sharp and Matassi, Curr. Opin. Genet. Dev. 4:851 (1994), Kane, Curr. Opin. Biotechnol. 6:494 (1995), and Makrides, Microbiol Rev. 60:512 (1996). As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed herein serve as a template for optimizing expression of polynucleotides in various cell types and species commonly used in the art and disclosed herein. Sequences containing preferential codons can be tested and optimized for expression in various species, and tested for functionality as disclosed herein.

A pG6b-encoding cDNA can be isolated by a variety of methods, such as by probing with a complete or partial human cDNA or with one or more sets of degenerate probes based on the disclosed sequences. A cDNA can also be cloned using the polymerase chain reaction with primers designed from the representative human pG6b sequences disclosed herein. In addition, a cDNA library can be used to transform or transfect host cells, and expression of the cDNA of interest can be detected with an antibody to pG6b polypeptide.

Those skilled in the art will recognize that the sequence disclosed in SEQ ID NO:1 represents a single allele of human pG6b, and that allelic variation and alternative splicing are expected to occur. Allelic variants of this sequence can be cloned by probing cDNA or genomic libraries from different individuals according to standard procedures. Allelic variants of the nucleotide sequences disclosed herein, including those containing silent mutations and those in which mutations result in amino acid sequence changes, are within the scope of the present invention, as are proteins which are allelic variants of the amino acid sequences disclosed herein. cDNA molecules generated from alternatively spliced mRNAs, which retain the properties of the pG6b polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Using the methods discussed above, one of ordinary skill in the art can prepare a variety of polypeptides that comprise a soluble pG6b receptor that is substantially homologous to SEQ ID NO:2, or that encodes amino acids of SEQ ID NO:3 or 4, or allelic variants thereof and retain the counter-receptor-binding properties of the wild-type pG6b receptor. Such polypeptides may also include additional polypeptide segments as generally disclosed herein.

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules comprising the nucleotide sequence of SEQ ID NO:1, or to nucleic acid molecules comprising a nucleotide sequence complementary to SEQ ID NO: 1, or fragments thereof.

In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Following hybridization, the nucleic acid molecules can be washed to remove non-hybridized nucleic acid molecules under stringent conditions, or under highly stringent conditions. See, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user-defined criteria. It is well within the abilities of one skilled in the art to adapthybridization and wash conditions for use with a particular polynucleotide hybrid.

The present invention also provides isolated pG6b polypeptides that have a substantially similar sequence identity to the polypeptides of SEQ ID NO:2 or 3, or their orthologs. The term “substantially similar sequence identity” is used herein to denote polypeptides having at least 70%, at least 80%, at least 90%, at least 95%, such as 96%, 97%, 98%, or greater than 95% sequence identity to the sequences shown in SEQ ID NO:3, or their orthologs. For example, variant and orthologous pG6b receptors can be used to generate an immune response and raise cross-reactive antibodies to human pG6b. Such antibodies can be humanized, and modified as described herein, and used therapeutically to treat psoriasis, psoriatic arthritis, IBD, colitis, endotoxemia as well as in other therapeutic applications described herein.

The present invention also contemplates pG6b variant nucleic acid molecules that can be identified using two criteria: a determination of the similarity between the encoded polypeptide with the amino acid sequence of SEQ ID NO:2, and a hybridization assay. Such pG6b variants include nucleic acid molecules (1) that remain hybridized with a nucleic acid molecule having the nucleotide sequence of SEQ ID NO:1 (or its complement) under stringent washing conditions, in which the wash stringency is equivalent to 0.5×-2×SSC with 0.1% SDS at 55-65° C., and (2) that encode a polypeptide having at least 70%, at least 80%, at least 90%, at least 95%, or greater than 95% such as 96%, 97%, 98%, or 99%, sequence identity to the amino acid sequence of SEQ ID NO:3. Alternatively, pG6b variants can be characterized as nucleic acid molecules (1) that remain hybridized with a nucleic acid molecule having the nucleotide sequence of SEQ ID NO: 1 (or its complement) under highly stringent washing conditions, in which the wash stringency is equivalent to 0.1×-0.2×SSC with 0.1% SDS at 50-65° C., and (2) that encode a polypeptide having at least 70%, at least 80%, at least 90%, at least 95% or greater than 95%, such as 96%, 97%, 98%, or 99% or greater, sequence identity to the amino acid sequence of SEQ ID NO:2.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes). The percent identity is then calculated as: ([Total number of identical matches]/[length of the longer sequence plus the number of gaps introduced into the longer sequence in order to align the two sequences])(100).

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative pG6b variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:2 or SEQ ID NO:3) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

The present invention includes nucleic acid molecules that encode a polypeptide having a conservative amino acid change, compared with an amino acid sequence disclosed herein. For example, variants can be obtained that contain one or more amino acid substitutions of SEQ ID NO:2, in which an alkyl amino acid is substituted for an alkyl amino acid in a pG6b amino acid sequence, an aromatic amino acid is substituted for an aromatic amino acid in a pG6b amino acid sequence, a sulfur-containing amino acid is substituted for a sulfur-containing amino acid in a pG6b amino acid sequence, a hydroxy-containing amino acid is substituted for a hydroxy-containing amino acid in a pG6b amino acid sequence, an acidic amino acid is substituted for an acidic amino acid in a pG6b amino acid sequence, a basic amino acid is substituted for a basic amino acid in a pG6b amino acid sequence, or a dibasic monocarboxylic amino acid is substituted for a dibasic monocarboxylic amino acid in a pG6b amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3). Particular variants of pG6b are characterized by having at least 70%, at least 80%, at least 90%, at least 95% or greater than 95% such as 96%, 97%, 98%, or 99% or greater sequence identity to the corresponding amino acid sequence (e.g., SEQ ID NO:3), wherein the variation in amino acid sequence is due to one or more conservative amino acid substitutions.

Conservative amino acid changes in a pG6b gene can be introduced, for example, by substituting nucleotides for the nucleotides recited in SEQ ID NO:1. Such “conservative amino acid” variants can be obtained by oligonucleotide-directed mutagenesis, linker-scanning mutagenesis, mutagenesis using the polymerase chain reaction, and the like (see Ausubel (1995); and McPherson (ed.), Directed Mutagenesis: A Practical Approach (IRL Press 1991)). A variant pG6b polypeptide can be identified by the ability to specifically bind anti-pG6b antibodies.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for pG6b amino acid residues.

Essential amino acids in the polypeptides of the present invention can be identified according to procedures known in the art, such as site-directed mutagenesis or alanine-scanning mutagenesis (Cunningham and Wells, Science 244:1081 (1989), Bass et al., Proc. Nat'l Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity to identify amino acid residues that are critical to the activity of the molecule. See also, Hilton et al., J. Biol. Chem. 271:4699 (1996).

Although sequence analysis can be used to further define the pG6b counter-receptor binding region, amino acids that play a role in pG6b binding activity (such as binding of pG6b to its counter-receptor or counter-receptors, or to an anti-pG6b antibody) can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306 (1992), Smith et al, J. Mol. Biol. 224:899 (1992), and Wlodaver et al., FEBS Lett. 309:59 (1992).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832, 1991, Ladner et al., U.S. Pat. No. 5,223,409, Huse, International Publication No. WO 92/06204, and region-directed mutagenesis (Derbyshire et al., Gene 46:145, 1986, and Ner et al., DNA 7:127, 1988). Moreover, pG6b labeled with biotin or FITC can be used for expression cloning of pG6b counter-receptors.

Variants of the disclosed pG6b nucleotide and polypeptide sequences can also be generated through DNA shuffling as disclosed by Stemmer, Nature 370:389 (1994), Stemmer, Proc. Nat'l Acad. Sci. USA 91:10747, 1994, and International Publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-pG6b antibodies, can be recovered from the host cells and rapidly sequenced using modern equipment. These methods allow the rapid determination of the importance of individual amino acid residues in a polypeptide of interest, and can be applied to polypeptides of unknown structure.

The present invention also includes “functional fragments” of pG6b polypeptides and nucleic acid molecules encoding such functional fragments. Routine deletion analyses of nucleic acid molecules can be performed to obtain functional fragments of a nucleic acid molecule that encodes a pG6b polypeptide. As an illustration, DNA molecules having the nucleotide sequence of SEQ ID NO: 1 can be digested with Bal31 nuclease to obtain a series of nested deletions. The fragments are then inserted into expression vectors in proper reading frame, and the expressed polypeptides are isolated and tested for the ability to bind anti-pG6b antibodies. One alternative to exonuclease digestion is to use oligonucleotide-directed mutagenesis to introduce deletions or stop codons to specify production of a desired fragment. Alternatively, particular fragments of a pG6b gene can be synthesized using the polymerase chain reaction.

This general approach is exemplified by studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113, 1993, Content et al., “Expression and preliminary deletion analysis of the 42 kDa 2-5 A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol 1, Boynton et al., (eds.) pages 169-199 (Academic Press 1985), Coumailleau et al., J. Biol. Chem. 270:29270, 1995; Fukunaga et al., J. Biol. Chem. 270:25291, 1995; Yamaguchi et al., Biochem. Pharmacol. 50:1295, 1995, and Meisel et al., Plant Molec. Biol. 30:1, 1996.

The present invention also contemplates functional fragments of a pG6b gene that have amino acid changes, compared with an amino acid sequence disclosed herein. A variant pG6b gene can be identified on the basis of structure by determining the level of identity with disclosed nucleotide and amino acid sequences, as discussed above. An alternative approach to identifying a variant gene on the basis of structure is to determine whether a nucleic acid molecule encoding a potential variant pG6b gene can hybridize to a nucleic acid molecule comprising a nucleotide sequence, such as SEQ ID NO:1.

The present invention also includes using functional fragments of pG6b polypeptides, antigenic epitopes, epitope-bearing portions of pG6b polypeptides, and nucleic acid molecules that encode such functional fragments, antigenic epitopes, epitope-bearing portions of pG6b polypeptides. Such fragments are used to generate polypeptides for use in generating antibodies and binding partners that agonize, bind, block, inhibit, increase, reduce, antagonize or neutralize activity of a B7 receptor. A “functional” pG6b polypeptide or fragment thereof as defined herein is characterized by its ability to block, inhibit, reduce, antagonize or neutralize inflammatory, proliferative or differentiating activity, by its ability to induce or inhibit specialized cell functions, or by its ability to bind specifically to an anti-pG6b antibody, cell, or B7 counter-receptor. As previously described herein, pG6b is characterized as a B7 family member by its receptor structure and domains as described herein. Thus, the present invention further contemplates using fusion proteins encompassing: (a) polypeptide molecules comprising one or more of the domains described above; and (b) functional fragments comprising one or more of these domains. The other polypeptide portion of the fusion protein may be contributed by another CD28 family receptor, such as CD28, CTLA-4, ICOS, PD-1 or BTLA, or by a non-native and/or an unrelated secretory signal peptide that facilitates secretion of the fusion protein.

The present invention also provides polypeptide fragments or peptides comprising an epitope-bearing portion of a pG6b polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, e.g., Geysen et al., Proc. Nat'l Acad. Sci. USA 81:3998, 1983).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660, 1983). Accordingly, antigenic epitope-bearing peptides, antigenic peptides, epitopes, and polypeptides of the present invention are useful to raise antibodies that bind with the polypeptides described herein, as well as to identify and screen anti-pG6b monoclonal antibodies that are neutralizing, and that may agonize, bind, block, inhibit, reduce, antagonize or neutralize the activity of its counter-receptor. Such neutralizing monoclonal antibodies of the present invention can bind to a pG6b antigenic epitope. Hopp/Woods hydrophilicity profiles can be used to determine regions that have the most antigenic potential within SEQ ID NO:3 (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. In pG6b these regions can be determined by one of skill in the art. Moreover, pG6b antigenic epitopes within SEQ ID NO:2 as predicted by a Jameson-Wolf plot, e.g., using DNASTAR Protean program (DNASTAR, Inc., Madison, Wis.) serve as preferred antigenic epitopes, and can be determined by one of skill in the art. Such antigenic epitopes include (1) amino acid residues 21 to 31 of SEQ ID NO:2; (2) amino acid residues 55 to 61 of SEQ ID NO:2; (3) amino acid residues 110 to 116 of SEQ ID NO:2; (4) amino acid residues 184 to 206 of SEQ ID NO:2; and (5) amino acid residues 191 to 197 of SEQ ID NO:2. In preferred embodiments, antigenic epitopes to which neutralizing antibodies of the present invention bind would contain residues of SEQ ID NO:2 (and corresponding residues of SEQ ID NO:3) that are important to counter-receptor binding.

Antigenic epitope-bearing peptides and polypeptides can contain at least four to ten amino acids, at least ten to fifteen amino acids, or about 15 to about 30 amino acids of an amino acid sequence disclosed herein. Such epitope-bearing peptides and polypeptides can be produced by fragmenting a pG6b polypeptide, or by chemical peptide synthesis, as described herein. Moreover, epitopes can be selected by phage display of random peptide libraries (see, e.g., Lane and Stephen, Curr. Opin. Immunol. 5:268, 1993; and Cortese et al., Curr. Opin. Biotechnol. 7:616, 1996). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et at. (eds.), Current Protocols in Immunology, pages 9.3.1-9.3.5 and pages 9.4.1-9.4.11 (John Wiley & Sons 1997).

For any pG6b polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise pG6b variants based upon the nucleotide and amino acid sequences described herein.

### 5. Production of pG6b Polypeptides

The polypeptides of the present invention, including full-length polypeptides; soluble monomeric, homodimeric, heterodimeric and multimeric receptors; full-length receptors; receptor fragments (e.g. counter-receptor-binding fragments and antigenic epitopes); functional fragments; and fusion proteins, can be produced in recombinant host cells following conventional techniques. To express a pG6b gene, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene which is suitable for selection of cells that carry the expression vector.

Expression vectors that are suitable for production of a foreign protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence. As discussed above, expression vectors can also include nucleotide sequences encoding a secretory sequence that directs the heterologous polypeptide into the secretory pathway of a host cell. For example, a pG6b expression vector may comprise a pG6b gene and a secretory sequence derived from any secreted gene.

pG6b proteins of the present invention may be expressed in mammalian cells. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (1H-3T3; ATCC CRL 1658).

For a mammalian host, the transcriptional and translational regulatory signals may be derived from mammalian viral sources, for example, adenovirus, bovine papilloma virus, simian virus, or the like, in which the regulatory signals are associated with a particular gene which has a high level of expression. Suitable transcriptional and translational regulatory sequences also can be obtained from mammalian genes, for example, actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene (Hamer et al., J. Molec. Appl Genet. 1:273, 1982), the TK promoter of Herpes virus (McKnight, Cell 31:355, 1982), the SV40 early promoter (Benoist et al., Nature 290:304, 1981), the Rous sarcoma virus promoter (Gorman et al., Proc. Nat'l Acad. Sci. USA 79:6777, 1982), the cytomegalovirus promoter (Foecking et al., Gene 45:101, 1980), and the mouse mammary tumor virus promoter (see generally Etcheverry, “Expression of Engineered Proteins in Mammalian Cell Culture,” in Protein Engineering: Principles and Practice, Cleland et al. (eds.), pages 163-181 (John Wiley & Sons, Inc. 1996)).

Alternatively, a prokaryotic promoter, such as the bacteriophage T3 RNA polymerase promoter, can be used to control pG6b gene expression in mammalian cells if the prokaryotic promoter is regulated by a eukaryotic promoter (Zhou et al., Mol. Cell. Biol. 10:4529, 1990; and Kaufman et al., Nucl Acids Res. 19:4485, 1991).

In certain embodiments, a DNA sequence encoding a pG6b soluble receptor polypeptide, or a fragment of pG6b polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers. Multiple components of a soluble receptor complex can be co-transfected on individual expression vectors or be contained in a single expression vector. Such techniques of expressing multiple components of protein complexes are well known in the art.

An expression vector can be introduced into host cells using a variety of standard techniques including calcium phosphate transfection, liposome-mediated transfection, microprojectile-mediated delivery, electroporation, and the like. The transfected cells can be selected and propagated to provide recombinant host cells that comprise the expression vector stably integrated in the host cell genome. Techniques for introducing vectors into eukaryotic cells and techniques for selecting such stable transformants using a dominant selectable marker are described, for example, by Ausubel (1995) and by Murray (ed.), Gene Transfer and Expression Protocols (Humana Press 1991).

For example, one suitable selectable marker is a gene that provides resistance to the antibiotic neomycin. In this case, selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A suitable amplifiable selectable marker is dihydrofolate reductase (DHFR), which confers resistance to methotrexate. Other drug resistance genes (e.g., hygromycin resistance, multi-drug resistance, puromycin acetyltransferase) can also be used. Alternatively, markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

pG6b polypeptides can also be produced by cultured mammalian cells using a viral delivery system. Exemplary viruses for this purpose include adenovirus, retroviruses, herpesvirus, vaccinia virus and adeno-associated virus (AAV). Adenovirus, a double-stranded DNA virus, is currently the best studied gene transfer vector for delivery of heterologous nucleic acid (for a review, see Becker et al., Meth. Cell Biol. 43:161, 1994; and Douglas and Curiel, Science & Medicine 4:44, 1997). Advantages of the adenovirus system include the accommodation of relatively large DNA inserts, the ability to grow to high-titer, the ability to infect a broad range of mammalian cell types, and flexibility that allows use with a large number of available vectors containing different promoters.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. An option is to delete the essential E1 gene from the viral vector, which results in the inability to replicate unless the E1 gene is provided by the host cell. Adenovirus vector-infected human 293 cells (ATCC Nos. CRL-1573, 45504, 45505), for example, can be grown as adherent cells or in suspension culture at relatively high cell density to produce significant amounts of protein (see Garnier et al., Cytotechnol. 15:145, 1994).

pG6b can also be expressed in other higher eukaryotic cells, such as avian, fungal, insect, yeast, or plant cells. The baculovirus system provides an efficient means to introduce cloned pG6b genes into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow et al., J. Virol. 67:4566, 1993). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the pG6b polypeptide into a baculovirus genome maintained in E. coli as a large plasmid called a “bacmid.” See Hill-Perkins and Possee, J. Gen. Virol. 71:971, 1990; Bonning et al., J. Gen. Virol. 75:1551, 1994; and Chazenbalk and Rapoport, J. Biol. Chem. 270:1543, 1995. In addition, transfer vectors can include an in-frame fusion with DNA encoding an epitope tag at the C- or N-terminus of the expressed pG6b polypeptide, for example, a Glu-Glu epitope tag (Grussenmeyer et al., Proc. Nat'l Acad. Sci. 82:7952, 1985). Using a technique known in the art, a transfer vector containing a pG6b gene is transformed into E. coli, and screened for bacmids which contain an interrupted lacZ gene indicative of recombinant baculovirus. The bacmid DNA containing the recombinant baculovirus genome is then isolated using common techniques.

The illustrative PFASTBAC vector can be modified to a considerable degree. For example, the polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins (see, e.g., Hill-Perkins and Possee, J. Gen. Virol. 71:971, 1990; Bonning et al., J. Gen. Virol. 75:1551, 1994; and Chazenbalk and Rapoport, J. Biol. Chem. 270:1543, 1995. In such transfer vector constructs, a short or long version of the basic protein promoter can be used. Moreover, transfer vectors can be constructed which replace the native pG6b secretory signal sequences with secretory signal sequences derived from insect proteins. For example, a secretory signal sequence from Ecdysteroid Glucosyltransferase (EGT), honey bee Melittin (Invitrogen Corporation; Carlsbad, Calif.), or baculovirus gp67 (PharMingen: San Diego, Calif.) can be used in constructs to replace the native pG6b secretory signal sequence.

The recombinant virus or bacmid is used to transfect host cells. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. When recombinant virus is used, the cells are typically grown up from an inoculation density of approximately 2-5×10⁵ cells to a density of 1-2×10⁶ cells at which time a recombinant viral stock is added at a multiplicity of infection (MOI) of 0.1 to 10, more typically near 3.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et at. (eds.), pages 183-218 (John Wiley & Sons, Inc. 1996).

Fungal cells, including yeast cells, can also be used to express the genes described herein. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp 17, YEp vectors such as YEp 13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). A suitable vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, e.g., Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A suitable selectable marker for use in Pichia methanolica is a P. methanolica ADE2 gene, which encodes phosphoribosyl-5-aminoimidazole carboxylase (AIRC; EC 4.1.1.21), and which allows ade2 host cells to grow in the absence of adenine. For large-scale, industrial processes where it is desirable to minimize the use of methanol, host cells can be used in which both methanol utilization genes (AUG1 and AUG2) are deleted. For production of secreted proteins, host cells can be deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Expression vectors can also be introduced into plant protoplasts, intact plant tissues, or isolated plant cells. Methods for introducing expression vectors into plant tissue include the direct infection or co-cultivation of plant tissue with Agrobacterium tumefaciens, microprojectile-mediated delivery, DNA injection, electroporation, and the like. See, e.g., Horsch et al., Science 227:1229, 1985; Klein et al., Biotechnology 10:268, 1992; and Miki et al., “Procedures for Introducing Foreign DNA into Plants,” in Methods in Plant Molecular Biology and Biotechnology, Glick et al. (eds.), pages 67-88 (CRC Press, 1993).

Alternatively, pG6b genes can be expressed in prokaryotic host cells. Suitable promoters that can be used to express pG6b polypeptides in a prokaryotic host are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters have been reviewed by Glick, J. Ind. Microbiol. 1:277 (1987), Watson et al., Molecular Biology of the Gene, 4th Ed. (Benjamin Cummins 1987), and by Ausubel et al. (1995).

Suitable prokaryotic hosts include E. coli and Bacillus subtilus. Suitable strains of E. coli include BL21(DE3), BL21(DE3)pLysS, BL21(DE3)pLysE, DH1, DH41, DH5, DH51, DH51F′, DH5IMCR, DH10B, DH10B/p3, DH 11S, C600, HB101, JM101, JM105, JM109, JM110, K38, RR1, Y1088, Y1089, CSH18, ER1451, and ER1647 (see, for example, Brown (ed.), Molecular Biology Labfax (Academic Press 1991)). Suitable strains of Bacillus subtilus include BR151, YB886, MI119, MI120, and B170 (see, e.g., Hardy, “Bacillus Cloning Methods,” in DNA Cloning: A Practical Approach, Glover (ed.) (IRL Press 1985)).

When expressing a pG6b polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et at. (eds.), page 15 (Oxford University Press 1995), Ward et al., “Genetic Manipulation and Expression of Antibodies,” in Monoclonal Antibodies: Principles and Applications, page 137 (Wiley-Liss, Inc. 1995), and Georgiou, “Expression of Proteins in Bacteria,” in Protein Engineering: Principles and Practice, Cleland et at. (eds.), page 101 (John Wiley & Sons, Inc. 1996)).

Standard methods for introducing expression vectors into bacterial, yeast, insect, and plant cells are provided, for example, by Ausubel (1995).

General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Standard techniques for recovering protein produced by a bacterial system is provided by, for example, Grisshammer et al., “Purification of over-produced proteins from E. coli cells,” in DNA Cloning 2: Expression Systems, 2nd Edition, Glover et at. (eds.), pages 59-92 (Oxford University Press 1995). Established methods for isolating recombinant proteins from a baculovirus system are described by Richardson (ed.), Baculovirus Expression Protocols (The Humana Press, Inc. 1995).

As an alternative, polypeptides of the present invention can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, e.g., Dawson et al., Science 266:776, 1994; Hackeng et al., Proc. Nat'l Acad. Sci. USA 94:7845, 1997; Dawson, Methods Enzymol. 287: 34, 1997; Muir et al., Proc. Nat'l Acad. Sci. USA 95:6705, 1998; and Severinov and Muir, J. Biol. Chem. 273:16205, 1998).

Peptides and polypeptides of the present invention comprise at least six, at least nine, or at least 15 contiguous amino acid residues of SEQ ID NO:2. As an illustration, polypeptides can comprise at least six, at least nine, or at least 15 contiguous amino acid residues of SEQ ID NO:2. Within certain embodiments of the invention, the polypeptides comprise 20, 30, 40, 50, 100, or more contiguous residues of these amino acid sequences. Nucleic acid molecules encoding such peptides and polypeptides are useful as polymerase chain reaction primers and probes.

Moreover, pG6b polypeptides and fragments thereof can be expressed as monomers, homodimers, heterodimers, or multimers within higher eukaryotic cells. Such cells can be used to produce pG6b monomeric, homodimeric, heterodimeric and multimeric receptor polypeptides that comprise at least one pG6b polypeptide (“pG6b-comprising receptors” or “pG6b-comprising receptor polypeptides”), or can be used as assay cells in screening systems. Within one aspect of the present invention, a polypeptide of the present invention comprising the pG6b extracellular domain is produced by a cultured cell, and the cell is used to screen for counter-receptors for the receptor, including a natural counter-receptor, as well as agonists and antagonists of the natural counter-receptor. To summarize this approach, a cDNA or gene encoding the receptor is combined with other genetic elements required for its expression (e.g., a transcription promoter), and the resulting expression vector is inserted into a host cell. Cells that express the DNA and produce functional receptor are selected and used within a variety of screening systems. Each component of the monomeric, homodimeric, heterodimeric and multimeric receptor complex can be expressed in the same cell. Moreover, the components of the monomeric, homodimeric, heterodimeric and multimeric receptor complex can also be fused to a transmembrane domain or other membrane fusion moiety to allow complex assembly and screening of transfectants as described above.

To assay the pG6b agonist and/or antagonist polypeptides and antibodies of the present invention, mammalian cells suitable for use in expressing pG6b-comprising receptors and transducing a receptor-mediated signal include cells that express other receptor subunits that may form a functional complex with pG6b (or pG6bRA). Within a preferred embodiment, the cell is dependent upon an exogenously supplied hematopoietic growth factor for its proliferation. Preferred cell lines of this type are the human TF-1 cell line (ATCC number CRL-2003) and the AML-193 cell line (ATCC number CRL-9589), which are GM-CSF-dependent human leukemic cell lines and BaF3 (Palacios and Steinmetz, Cell 41: 727-734, 1985) which is an IL-3 dependent murine pre-B cell line. Other cell lines include BHK, COS-1, and CHO cells. Suitable host cells can be engineered to produce the necessary receptor subunits or other cellular component needed for the desired cellular response. This approach is advantageous because cell lines can be engineered to express receptor subunits from any species, thereby overcoming potential limitations arising from species specificity. Species orthologs of the human receptor cDNA can be cloned and used within cell lines from the same species, such as a mouse cDNA in the BaF3 cell line.

Cells expressing functional receptor are used within screening assays. A variety of suitable assays are known in the art. These assays are based on the detection of a biological response in a target cell. One such assay is a cell proliferation assay. Cells are cultured in the presence or absence of a test compound, and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, (1983)). An alternative assay format uses cells that are further engineered to express a reporter gene. The reporter gene is linked to a promoter element that is responsive to the receptor-linked pathway, and the assay detects activation of transcription of the reporter gene. A preferred promoter element in this regard is a serum response element, or SRE. See, e.g., Shaw et al., Cell 56:563-572, 1989. A preferred such reporter gene is a luciferase gene (de Wet et al., Mol. Cell. Biol. 7:725, 1987). Expression of the luciferase gene is detected by luminescence using methods known in the art (e.g., Baumgartner et al., J. Biol. Chem. 269:29094-29101, 1994; Schenborn and Goiffin, Promega Notes 41:11, 1993). Luciferase activity assay kits are commercially available from, for example, Promega Corp., Madison, Wis. Target cell lines of this type can be used to screen libraries of chemicals, cell-conditioned culture media, fungal broths, soil samples, water samples, and the like. For example, a bank of cell-conditioned media samples can be assayed on a target cell to identify cells that produce counter-receptor. Positive cells are then used to produce a cDNA library in a mammalian expression vector, which is divided into pools, transfected into host cells, and expressed. Media samples from the transfected cells are then assayed, with subsequent division of pools, re-transfection, subculturing, and re-assay of positive cells to isolate a cloned cDNA encoding the counter-receptor.

Several pG6b responsive cell lines are known in the art or can be constructed, for example, the Baf3/DIRS1/cytoR11 cell line (WIPO Publication No. WO 02/072607). Moreover several IL-22 responsive cell lines are known (Dumontier et al., J. Immunol. 164:1814-1819, 2000; Dumoutier, L. et al., Proc. Nat'l Acad. Sci. 97:10144-10149, 2000; Xie M H et al., J. Biol. Chem. 275: 31335-31339, 2000; Kotenko S V et al., J. Biol. Chem. 276:2725-2732, 2001), as well as those that express the IL-22 receptor subunit pG6b. For example, the following cells are responsive to IL-22: TK-10 (Xie M H et al., supra) (human renal carcinoma); SW480 (ATCC No. CCL-228) (human colon adenocarcinoma); HepG2 (ATCC No. HB-8065) (human hepatoma); PC12 (ATCC No. CRL-1721) (murine neuronal cell model; rat pheochromocytoma); and MES13 (ATCC No. CRL-1927) (murine kidney mesangial cell line). In addition, some cell lines express pG6b (IL-22 receptor) are also candidates for responsive cell lines to IL-22: A549 (ATCC No. CCL-185) (human lung carcinoma); G-361 (ATCC No. CRL-1424) (human melanoma); and Caki-1 (ATCC No. HTB-46) (human renal carcinoma). In addition, IL-22-responsive cell lines can be constructed, for example, the Baf3/cytoR11/CRF2-4 cell line described herein (WIPO Publication No. WO 02/12345). These cells can be used in assays to assess the functionality of pG6b as an pG6b or IL-22 antagonist or anti-inflammatory factor.

### 6. Production of pG6b Fusion Proteins and Conjugates

One general class of pG6b analogs are variants having an amino acid sequence that is a mutation of the amino acid sequence disclosed herein. Another general class of pG6b analogs is provided by anti-idiotype antibodies, and fragments thereof, as described below. Moreover, recombinant antibodies comprising anti-idiotype variable domains can be used as analogs (see, e.g., Monfardini et al., Proc. Assoc. Am. Physicians 108:420, 1996). Since the variable domains of anti-idiotype pG6b antibodies mimic pG6b, these domains can provide pG6b binding activity. Methods of producing anti-idiotypic catalytic antibodies are known to those of skill in the art (see, e.g., Joron et al, Ann. N.Y. Acad. Sci. 672:216, 1992; Friboulet et al., Appl Biochem. Biotechnol 47:229, 1994; and Avalle et al., Ann. NY Acad. Sci. 864:118, 1998).

Another approach to identifying pG6b analogs is provided by the use of combinatorial libraries. Methods for constructing and screening phage display and other combinatorial libraries are provided, for example, by Kay et al., Phage Display of Peptides and Proteins (Academic Press 1996), Verdine, U.S. Pat. No. 5,783,384, Kay, et. al, U.S. Pat. No. 5,747,334, and Kauffman et al., U.S. Pat. No. 5,723,323.

pG6b polypeptides have both in vivo and in vitro uses. As an illustration, a soluble form of pG6b can be added to cell culture medium to inhibit the effects of the pG6b counter-receptor produced by the cultured cells.

Fusion proteins of pG6b can be used to express pG6b in a recombinant host, and to isolate the produced pG6b. As described below, particular pG6b fusion proteins also have uses in diagnosis and therapy. One type of fusion protein comprises a peptide that guides a pG6b polypeptide from a recombinant host cell. To direct a pG6b polypeptide into the secretory pathway of a eukaryotic host cell, a secretory signal sequence (also known as a signal peptide, a leader sequence, prepro sequence or pre sequence) is provided in the pG6b expression vector. While the secretory signal sequence may be derived from pG6b, a suitable signal sequence may also be derived from another secreted protein or synthesized de novo. The secretory signal sequence is operably linked to a pG6b-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

Although the secretory signal sequence of pG6b or another protein produced by mammalian cells (e.g., tissue-type plasminogen activator signal sequence, as described, for example, in U.S. Pat. No. 5,641,655) is useful for expression of pG6b in recombinant mammalian hosts, a yeast signal sequence is preferred for expression in yeast cells. Examples of suitable yeast signal sequences are those derived from yeast mating phermone α-factor (encoded by the MFα1 gene), invertase (encoded by the SUC2 gene), or acid phosphatase (encoded by the PHO5 gene). See, e.g., Romanos et al., “Expression of Cloned Genes in Yeast,” in DNA Cloning 2: A Practical Approach, 2^{nd} Edition, Glover and Hames (eds.), pages 123-167 (Oxford University Press 1995).

pG6b soluble receptor polypeptides can be prepared by expressing a truncated DNA encoding the extracellular domain, for example, a polypeptide which contains SEQ ID NO:2, or the corresponding region of a non-human receptor. It is preferred that the extracellular domain polypeptides be prepared in a form substantially free of transmembrane and intracellular polypeptide segments. To direct the export of the receptor domain from the host cell, the receptor DNA is linked to a second DNA segment encoding a secretory peptide, such as a t-PA secretory peptide. To facilitate purification of the secreted receptor domain, a C-terminal extension, such as a poly-histidine tag, substance P, Flag™ peptide (Hopp et al., Biotechnology 6:1204-1210, (1988); available from Eastman Kodak Co., New Haven, Conn.) or another polypeptide or protein for which an antibody or other specific binding agent is available, can be fused to the receptor polypeptide. Moreover, pG6b antigenic epitopes from the extracellular cytokine binding domains are also prepared as described above.

In an alternative approach, a receptor extracellular domain of pG6b or other B7 receptor component can be expressed as a fusion with immunoglobulin heavy chain constant regions, typically an F_{c} fragment, which contains two constant region domains and a hinge region but lacks the variable region (See Sledziewski, et al., U.S. Pat. Nos. 6,018,026 and 5,750,375). The soluble pG6b polypeptides of the present invention include such fusions. Such fusions are typically secreted as multimeric molecules wherein the Fc portions are disulfide bonded to each other and two receptor polypeptides are arrayed in closed proximity to each other. Fusions of this type can be used to affinity purify the cognate counter-receptor from solution, as an in vitro assay tool, to block, inhibit or reduce signals in vitro by specifically titrating out counter-receptor, and as antagonists in vivo by administering them parenterally to bind circulating counter-receptor and clear it from the circulation. To purify counter-receptor, a pG6b-Ig chimera is added to a sample containing the counter-receptor (e.g., cell-conditioned culture media or tissue extracts) under conditions that facilitate receptor-counter-receptor binding (typically near-physiological temperature, pH, and ionic strength). The chimera-counter-receptor complex is then separated by the mixture using protein A, which is immobilized on a solid support (e.g., insoluble resin beads). The counter-receptor is then eluted using conventional chemical techniques, such as with a salt or pH gradient. In the alternative, the chimera itself can be bound to a solid support, with binding and elution carried out as above. The chimeras may be used in vivo to regulate inflammatory responses including acute phase responses such as serum amyloid A (SAA), C-reactive protein (CRP), and the like. Chimeras with high binding affinity are administered parenterally (e.g., by intramuscular, subcutaneous or intravenous injection). Circulating molecules bind counter-receptor and are cleared from circulation by normal physiological processes. For use in assays, the chimeras are bound to a support via the F_{c} region and used in an ELISA format.

To assist in isolating anti-pG6b and binding partners of the present invention, an assay system that uses a counter-receptor-binding receptor (or an antibody, one member of a complement/anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson (J. Immunol. Methods 145:229-40, 1991) and Cunningham and Wells (J. Mol. Biol. 234:554-63, 1993). A receptor, antibody, member or fragment is covalently attached, using amine or sulfhydryl chemistry, to dextran fibers that are attached to gold film within the flow cell. A test sample is passed through the cell. If a counter-receptor, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding. Alternatively, counter-receptor/receptor binding can be analyzed using SELDI™ technology (Ciphergen, Inc., Palo Alto, Calif.). Moreover, BIACORE technology, described above, can be used to be used in competition experiments to determine if different monoclonal antibodies bind the same or different epitopes on the pG6b polypeptide, and as such, be used to aid in epitope mapping of antibodies of the present invention.

Counter-receptor-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

The present invention further provides a variety of other polypeptide fusions and related multimeric proteins comprising one or more polypeptide fusions. For example, a soluble pG6b receptor can be prepared as a fusion to a dimerizing protein as disclosed in U.S. Pat. Nos. 5,155,027 and 5,567,584. Preferred dimerizing proteins in this regard include immunoglobulin constant region domains, e.g., IgGγI, and the human κ light chain. Immunoglobulin-soluble pG6b fusions can be expressed in genetically engineered cells to produce a variety of multimeric pG6b receptor analogs. Auxiliary domains can be fused to soluble pG6b receptor to target them to specific cells, tissues, or macromolecules (e.g., collagen, or cells expressing the pG6b counter-receptors). A pG6b polypeptide can be fused to two or more moieties, such as an affinity tag for purification and a targeting domain. Polypeptide fusions can also comprise one or more cleavage sites, particularly between domains. See Tuan et al., Connective Tissue Research 34:1-9, 1996.

In bacterial cells, it is often desirable to express a heterologous protein as a fusion protein to decrease toxicity, increase stability, and to enhance recovery of the expressed protein. For example, pG6b can be expressed as a fusion protein comprising a glutathione S-transferase polypeptide. Glutathione S-transferease fusion proteins are typically soluble, and easily purifiable from E. coli lysates on immobilized glutathione columns. In similar approaches, a pG6b fusion protein comprising a maltose binding protein polypeptide can be isolated with an amylose resin column, while a fusion protein comprising the C-terminal end of a truncated Protein A gene can be purified using IgG-Sepharose. Established techniques for expressing a heterologous polypeptide as a fusion protein in a bacterial cell are described, for example, by Williams et al., “Expression of Foreign Proteins in E. coli Using Plasmid Vectors and Purification of Specific Polyclonal Antibodies,” in DNA Cloning 2: A Practical Approach, 2^{nd} Edition, Glover and Hames (Eds.), pages 15-58 (Oxford University Press 1995). In addition, commercially available expression systems are available. For example, the PINPOINT Xa protein purification system (Promega Corporation; Madison, Wis.) provides a method for isolating a fusion protein comprising a polypeptide that becomes biotinylated during expression with a resin that comprises avidin.

Peptide tags that are useful for isolating heterologous polypeptides expressed by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies). See, for example, Luo et al., Arch. Biochem. Biophys. 329:215, 1996; Morganti et al., Biotechnol. Appl. Biochem. 23:67, 1996; and Zheng et al., Gene 186:55, 1997. Nucleic acid molecules encoding such peptide tags are available, for example, from Sigma-Aldrich Corporation (St. Louis, Mo.).

Another form of fusion protein comprises a pG6b polypeptide and an immunoglobulin heavy chain constant region, typically an F_{c} fragment, which contains two or three constant region domains and a hinge region but lacks the variable region. As an illustration, Chang et al., U.S. Pat. No. 5,723,125, describe a fusion protein comprising a human interferon and a human immunoglobulin F_{c} fragment. The C-terminal of the interferon is linked to the N-terminal of the F_{c} fragment by a peptide linker moiety. An example of a peptide linker is a peptide comprising primarily a T cell inert sequence, which is immunologically inert. In this fusion protein, an illustrative F_{c} moiety is a human γ4 chain, which is stable in solution and has little or no complement activating activity. Accordingly, the present invention contemplates a pG6b fusion protein that comprises a pG6b moiety and a human F_{c} fragment, wherein the C-terminus of the pG6b moiety is attached to the N-terminus of the F_{c} fragment via a peptide linker. The pG6b moiety can be a pG6b molecule or a fragment thereof. For example, a fusion protein can comprise the amino acid of SEQ ID NO:3 and an F_{c} fragment (e.g., a human F_{c} fragment).

In another variation, a pG6b fusion protein comprises an IgG sequence, a pG6b moiety covalently joined to the amino terminal end of the IgG sequence, and a signal peptide that is covalently joined to the amino terminal of the pG6b moiety, wherein the IgG sequence consists of the following elements in the following order: a hinge region, a CH₂ domain, and a CH₃ domain. Accordingly, the IgG sequence lacks a CH₁ domain. The pG6b moiety displays a pG6b activity, as described herein, such as the ability to bind with a pG6b counter-receptor. This general approach to producing fusion proteins that comprise both antibody and nonantibody portions has been described by LaRochelle et al., EP 742830 (WO 95/21258).

Fusion proteins comprising a pG6b moiety and an Fc moiety can be used, for example, as an in vitro assay tool. For example, the presence of a pG6b counter-receptor in a biological sample can be detected using a pG6b-immunoglobulin fusion protein, in which the pG6b moiety is used to bind the counter-receptor, and a macromolecule, such as Protein A or anti-Fc antibody, is used to bind the fusion protein to a solid support. Such systems can be used to identify agonists and antagonists that interfere with the binding of pG6b to its counter-receptor.

Other examples of antibody fusion proteins include polypeptides that comprise an antigen-binding domain and a pG6b fragment that contains a pG6b extracellular domain. Such molecules can be used to target particular tissues for the benefit of pG6b binding activity.

The present invention further provides a variety of other polypeptide fusions. For example, part or all of a domain(s) conferring a biological function can be swapped between pG6b of the present invention with the functionally equivalent domain(s) from another member of the cytokine receptor family. Polypeptide fusions can be expressed in recombinant host cells to produce a variety of pG6b fusion analogs. A pG6b polypeptide can be fused to two or more moieties or domains, such as an affinity tag for purification and a targeting domain. Polypeptide fusions can also comprise one or more cleavage sites, particularly between domains. See, e.g., Tuan et al., Connective Tissue Research 34:1, 1996.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16-19 to 16-25.

pG6b binding domains can be further characterized by physical analysis of structure, as determined by such techniques as nuclear magnetic resonance, crystallography, electron diffraction or photoaffinity labeling, in conjunction with mutation of putative contact site amino acids of pG6b counter-receptor agonists. See, e.g., de Vos et al., Science 255:306, 1992; Smith et al., J. Mol. Biol. 224:899, 1992; and Wlodaver et al., FEBS Lett. 309:59, 1992.

The present invention also contemplates chemically modified pG6b compositions, in which a pG6b polypeptide is linked with a polymer. Illustrative pG6b polypeptides are soluble polypeptides that lack a functional transmembrane domain, such as a polypeptide consisting of amino acid residues SEQ ID NO:3. Typically, the polymer is water soluble so that the pG6b conjugate does not precipitate in an aqueous environment, such as a physiological environment. An example of a suitable polymer is one that has been modified to have a single reactive group, such as an active ester for acylation, or an aldehyde for alkylation. In this way, the degree of polymerization can be controlled. An example of a reactive aldehyde is polyethylene glycol propionaldehyde, or mono-(C1-C10) alkoxy, or aryloxy derivatives thereof (see, e.g., Harris, et al., U.S. Pat. No. 5,252,714). The polymer may be branched or unbranched. Moreover, a mixture of polymers can be used to produce pG6b conjugates.

pG6b conjugates used for therapy can comprise pharmaceutically acceptable water-soluble polymer moieties. Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A pG6b conjugate can also comprise a mixture of such water-soluble polymers.

One example of a pG6b conjugate comprises a pG6b moiety and a polyalkyl oxide moiety attached to the N-terminus of the pG6b moiety. PEG is one suitable polyalkyl oxide. As an illustration, pG6b can be modified with PEG, a process known as “PEGylation.” PEGylation of pG6b can be carried out by any of the PEGylation reactions known in the art (see, e.g., EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, pG6b conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with a pG6b polypeptide. An example of an activated PEG ester is PEG esterified to N-hydroxysuccinimide. As used herein, the term “acylation” includes the following types of linkages between pG6b and a water soluble polymer: amide, carbamate, urethane, and the like. Methods for preparing PEGylated pG6b by acylation will typically comprise the steps of (a) reacting a pG6b polypeptide with PEG (such as a reactive ester of an aldehyde derivative of PEG) under conditions whereby one or more PEG groups attach to pG6b, and (b) obtaining the reaction product(s). Generally, the optimal reaction conditions for acylation reactions will be determined based upon known parameters and desired results. For example, the larger the ratio of PEG:pG6b, the greater the percentage of polyPEGylated pG6b product.

The product of PEGylation by acylation is typically a polyPEGylated pG6b product, wherein the lysine ε-amino groups are PEGylated via an acyl linking group. An example of a connecting linkage is an amide. Typically, the resulting pG6b will be at least 95% mono-, di-, or tri-pegylated, although some species with higher degrees of PEGylation may be formed depending upon the reaction conditions. PEGylated species can be separated from unconjugated pG6b polypeptides using standard purification methods, such as dialysis, ultrafiltration, ion exchange chromatography, affinity chromatography, and the like.

PEGylation by alkylation generally involves reacting a terminal aldehyde derivative of PEG with pG6b in the presence of a reducing agent. PEG groups can be attached to the polypeptide via a —CH₂—NH group.

Moreover, anti-pG6b antibodies or antibody fragments of the present invention can be PEGylated using methods in the art and described herein.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the -amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups. The present invention provides a substantially homogenous preparation of pG6b monopolymer conjugates.

Reductive alkylation to produce a substantially homogenous population of monopolymer pG6b conjugate molecule can comprise the steps of: (a) reacting a pG6b polypeptide with a reactive PEG under reductive alkylation conditions at a pH suitable to permit selective modification of the α-amino group at the amino terminus of the pG6b, and (b) obtaining the reaction product(s). The reducing agent used for reductive alkylation should be stable in aqueous solution and able to reduce only the Schiff base formed in the initial process of reductive alkylation. Illustrative reducing agents include sodium borohydride, sodium cyanoborohydride, dimethylamine borane, trimethylamine borane, and pyridine borane.

For a substantially homogenous population of monopolymer pG6b conjugates, the reductive alkylation reaction conditions are those that permit the selective attachment of the water-soluble polymer moiety to the N-terminus of pG6b. Such reaction conditions generally provide for pKa differences between the lysine amino groups and the α-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer:pG6b need not be as large because more reactive groups are available. Typically, the pH will fall within the range of 3 to 9, or 3 to 6. This method can be employed for making pG6b-comprising homodimeric, heterodimeric or multimeric soluble receptor conjugates.

Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, or about 12 kDa to about 25 kDa. The molar ratio of water-soluble polymer to pG6b will generally be in the range of 1:1 to 100:1. Typically, the molar ratio of water-soluble polymer to pG6b will be 1:1 to 20:1 for polyPEGylation, and 1:1 to 5:1 for monoPEGylation.

General methods for producing conjugates comprising a polypeptide and water-soluble polymer moieties are known in the art. See, e.g., Karasiewicz et al., U.S. Pat. No. 5,382,657, Greenwald et al., U.S. Pat. No. 5,738,846, Nieforth et al., Clin. Pharmacol Ther. 59:636 (1996), Monkarsh et al., Anal Biochem. 247:434 (1997)). This method can be employed for making pG6b-comprising homodimeric, heterodimeric or multimeric soluble receptor conjugates.

The present invention contemplates compositions comprising a peptide or polypeptide, such as a soluble receptor or antibody described herein. Such compositions can further comprise a carrier. The carrier can be a conventional organic or inorganic carrier. Examples of carriers include water, buffer solution, alcohol, propylene glycol, macrogol, sesame oil, corn oil, and the like.

### 7. Isolation of pG6b Polypeptides

The polypeptides of the present invention can be purified to at least about 80% purity, to at least about 90% purity, to at least about 95% purity, or greater than 95%, such as 96%, 97%, 98%, or greater than 99% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. In certain preparations, purified polypeptide is substantially free of other polypeptides, particularly other polypeptides of animal origin.

Fractionation and/or conventional purification methods can be used to obtain preparations of pG6b purified from natural sources (e.g., human tissue sources), synthetic pG6b polypeptides, and recombinant pG6b polypeptides and fusion pG6b polypeptides purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

Additional variations in pG6b isolation and purification can be devised by those of skill in the art. For example, anti-pG6b antibodies, obtained as described below, can be used to isolate large quantities of protein by immunoaffinity purification.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529, 1990). Within additional embodiments of the invention, a fusion of the polypeptide of interest and an affinity tag (e.g., maltose-binding protein, an immunoglobulin domain) may be constructed to facilitate purification. Moreover, the counter-receptor-binding properties of pG6b extracellular domain can be exploited for purification, for example, of pG6b-comprising soluble receptors; for example, by using affinity chromatography wherein the appropriate counter-receptor is bound to a column and the pG6b-comprising receptor is bound and subsequently eluted using standard chromatography methods.

pG6b polypeptides or fragments thereof may also be prepared through chemical synthesis, as described above. pG6b polypeptides may be monomers or multimers; glycosylated or non-glycosylated; PEGylated or non-PEGylated; and may or may not include an initial methionine amino acid residue.

### 8. Production of Antibodies to pG6b Proteins

Antibodies to pG6b can be obtained, for example, using the product of a pG6b expression vector or pG6b isolated from a natural source as an antigen. Particularly useful anti-pG6b antibodies “bind specifically” with pG6b. Antibodies are considered to be specifically binding if the antibodies exhibit at least one of the following two properties: (1) antibodies bind to pG6b with a threshold level of binding activity, and (2) antibodies do not significantly cross-react with polypeptides related to pG6b.

With regard to the first characteristic, antibodies specifically bind if they bind to a pG6b polypeptide, peptide or epitope with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660, 1949). With regard to the second characteristic, antibodies do not significantly cross-react with related polypeptide molecules, for example, if they detect pG6b, but not presently known polypeptides using a standard Western blot analysis. Examples of known related polypeptides include known cytokine receptors.

Anti-pG6b antibodies can be produced using antigenic pG6b epitope-bearing peptides and polypeptides. Antigenic epitope-bearing peptides and polypeptides of the present invention contain a sequence of at least nine, or between 15 to about 30 amino acids contained within SEQ ID NO:2 or another amino acid sequence disclosed herein. However, peptides or polypeptides comprising a larger portion of an amino acid sequence of the invention, containing from 30 to 50 amino acids, or any length up to and including the entire amino acid sequence of a polypeptide of the invention, also are useful for inducing antibodies that bind with pG6b. It is desirable that the amino acid sequence of the epitope-bearing peptide is selected to provide substantial solubility in aqueous solvents (i.e., the sequence includes relatively hydrophilic residues, while hydrophobic residues are typically avoided). Moreover, amino acid sequences containing proline residues may be also be desirable for antibody production.

As an illustration, potential antigenic sites in pG6b were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549-586 (Plenum Press 1990), and Garnier-Robson, Garnier et al., J. Mol. Biol. 120:97 (1978) (Chou-Fasman parameters: conformation table=64 proteins; a region threshold=103; β region threshold=105; Garnier-Robson parameters: α and β decision constants=0). In the sixth subroutine, flexibility parameters and hydropathy/solvent accessibility factors were combined to determine a surface contour value, designated as the “antigenic index.” Finally, a peak broadening function was applied to the antigenic index, which broadens major surface peaks by adding 20, 40, 60, or 80% of the respective peak value to account for additional free energy derived from the mobility of surface regions relative to interior regions. This calculation was not applied, however, to any major peak that resides in a helical region, since helical regions tend to be less flexible.

The results of this analysis indicated that the following amino acid sequences of SEQ ID NO:2 would provide suitable antigenic peptides: Hopp/Woods hydrophilicity profiles can be used to determine regions that have the most antigenic potential within SEQ ID NO:3 (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Moreover, pG6b antigenic epitopes within SEQ ID NO:2 as predicted by a Jameson-Wolf plot, e.g., using DNASTAR Protean program (DNASTAR, Inc., Madison, Wis.) serve as preferred antigenic epitopes, and can be determined by one of skill in the art. Such antigenic epitopes include (1) amino acid residues 21 to 31 of SEQ ID NO:2; (2) amino acid residues 55 to 61 of SEQ ID NO:2; (3) amino acid residues 110 to 116 of SEQ ID NO:2; (4) amino acid residues 184 to 206 of SEQ ID NO:2; and (5) amino acid residues 191 to 197 of SEQ ID NO:2. The present invention contemplates the use of any one of antigenic peptides 1 to 5 to generate antibodies to pG6b or as a tool to screen or identify neutralizing monoclonal antibodies of the present invention. The present invention contemplates the use of any antigenic peptides or epitopes described herein to generate antibodies to pG6b, as well as to identify and screen anti-pG6b monoclonal antibodies that may bind, agonize, block, inhibit, reduce, increase, antagonize or neutralize the activity of a pG6b counter-receptor.

Polyclonal antibodies to recombinant pG6b protein or to pG6b isolated from natural sources can be prepared using methods well-known to those of skill in the art. See, e.g., Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of a pG6b polypeptide can be increased through the use of an adjuvant, such as alum (aluminum hydroxide) or Freund's complete or incomplete adjuvant. Polypeptides useful for immunization also include fusion polypeptides, such as fusions of pG6b or a portion thereof with an immunoglobulin polypeptide or with maltose binding protein. The polypeptide immunogen may be a full-length molecule or a portion thereof (e.g., the extracellular domain of pG6b or an antigenic fragment thereof). If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-pG6b antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal anti-pG6b antibodies can be generated. Rodent mono-clonal antibodies to specific antigens may be obtained by methods known to those skilled in the art (see, e.g., Kohler et al., Nature 256:495, 1975; Coligan et al. (eds.), Current Protocols in Immunology, Vol 1, pages 2.5.1-2.6.7 (John Wiley & Sons 1991) [“Coligan”]; Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a pG6b gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures. Particularly suitable immunogenic compositions for production of anti-pG6b antibodies include polypeptides comprising the extracellular domain of pG6b (e.g., SEQ ID NO:3) as well as antigenic fragments thereof.

In addition, an anti-pG6b antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13, 1994; Lonberg et al., Nature 368:856, 1994; and Taylor et al., Int. Immun. 6:579, 1994.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan at pages 2.7.1-2.7.12 and pages 2.9.1-2.9.3; Baines et al., “Purification of Immunoglobulin G (IgG),” in Methods in Molecular Biology, Vol 10, pages 79-104 (The Humana Press, Inc. 1992)).

For particular uses, it may be desirable to prepare fragments of anti-pG6b antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230, 1960; Porter, Biochem. J. 73:119, 1959; Edelman et al., in Methods in Enzymology Vol 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659, 1972. Alternatively, the variable chains can be linked by an intermolecular disulfide bond or cross-linked by chemicals such as glutaraldehyde (see, e.g., Sandhu, Crit. Rev. Biotech. 12:437, 1992).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97, 1991 (see also Bird et al., Science 242:423, 1988; Ladner et al., U.S. Pat. No. 4,946,778; Pack et al., Bio/Technology 11:1271, 1993, and Sandhu, supra).

As an illustration, a scFv can be obtained by exposing lymphocytes to pG6b polypeptide in vitro, and selecting antibody display libraries in phage or similar vectors (for instance, through use of immobilized or labeled pG6b protein or peptide). Genes encoding polypeptides having potential pG6b polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a counter-receptor or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the pG6b sequences disclosed herein to identify proteins which bind to pG6b.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, e.g., Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-pG6b antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833, 1989. Techniques for producing humanized monoclonal antibodies are described, for example, by Jones et al., Nature 321:522, 1986; Carter et al., Proc. Nat'l Acad. Sci. USA 89:4285, 1992; Sandhu, Crit. Rev. Biotech. 12:437, 1992; Singer et al., J. Immun. 150:2844, 1993; Sudhir (ed.), Antibody Engineering Protocols (Humana Press, Inc. 1995), Kelley, “Engineering Therapeutic Antibodies,” in Protein Engineering Principles and Practice, Cleland et al. (eds.), pages 399-434 (John Wiley & Sons, Inc. 1996), and by Queen et al., U.S. Pat. No. 5,693,762 (1997).

In particular embodiments of the invention, an anti-pG6b antibody is characterized by the following properties:
(a) the antibody specifically binds to the extracellular domain of pGB6; and
(b) the antibody, when covalently coupled to microbeads to yield an immobilized form the antibody, is capable of inhibiting TcR-mediated activation in a T cell in vitro, where the TcR-mediated activation includes contacting the T cell with an agonistic anti-CD3 antibody also coupled the microbeads.

Typically, the anti-pG6b antibody binds to the extracellular domain of human pG6b (SEQ ID NO:3) or mouse pG6b (SEQ ID NO:6). In certain variations, the anti-pG6b antibody is further characterized in that the immobilized form of the antibody is capable of inhibiting the TcR-mediated activation by at least about 50% relative to a control T cell that is contacted with the anti-CD3 covalently coupled to microbeads in the absence of the anti-pG6b antibody. In yet other variations, the anti-pG6b antibody is further characterized in that the immobilized form of the antibody is capable of inhibiting TcR-mediated activation comprising contacting the T cell with the anti-CD3 antibody covalently coupled to microbeads and a soluble, agonistic anti-CD28 antibody. In a specific variation, the anti-pG6b antibody has T cell inhibitory activity when coupled to beads approximately equal to that observed for bead-coupled anti-CTLA-4 mAb (e.g., R&D Systems, clone #48815, catalog number MAB325). T cell activation can be assessed, for example, with respect to particular T cell subpopulations, typically CD4⁺ or CD8⁺ T cells. Suitable microbeads for covalent coupling of antibodies to assess T cell activation include, for example, latex or paramagnetic beads (e.g., tosylactivated beads, such as commercially available from Dynal Biotech ASA (Oslo, Norway)).

In other embodiments, an anti-pG6b antibody is characterized by the following properties:
(1) the antibody specifically binds to the extracellular domain of a pG6b protein; and
(2) the antibody, in a soluble form,
   (i) is capable of inhibiting TcR-mediated activation in a T cell in vitro, where the TcR-mediated activation includes contacting the T cell with a soluble, agonistic anti-CD3 antibody in the absence CD28-mediated co-stimulation; and/or
   (ii) is capable of enhancing TcR-mediated activation in a T cell in vitro, where the TcR-mediated activation includes contacting the second T cell with the anti-CD3 antibody and a soluble, agonistic anti-CD28 antibody.

In typical variations, the anti-pG6b antibody binds to the extracellular domain of human pG6b (SEQ ID NO:3) or mouse pG6b (SEQ ID NO:6). In certain embodiments, the antibody is characterized by both (i) and (ii) above. Preferably, where the soluble form of the antibody is capable of inhibiting TcR-mediated activation in an anti-CD3-stimulated T cell in vitro, the soluble antibody is capable of inhibiting the TcR-mediated activation by at least about 50% relative to a control T cell that is contacted with the soluble anti-CD3 antibody in the absence of CD28-mediated co-stimulation and in the absence of the anti-pG6b antibody; for example, in particular variations, the inhibition of TcR-mediated activation is between about 50% and about 90%, and in a specific embodiment the inhibition is about 90%. Further, where the soluble antibody is capable of enhancing TcR-mediated activation in an anti-CD3/anti-CD28-stimulated T cell in vitro, where the TcR-mediated activation includes contacting the T cell with the soluble anti-CD3 and anti-CD28 antibodies, the soluble anti-pG6b antibody is typically capable of enhancing the TcR-mediated activation by at least about 20% relative to a control T cell that is contacted with both the anti-CD3 antibody and the anti-CD28 antibody in the absence of the anti-pG6b antibody; for example, in some variations, the enhancement of TcR-mediated activation is between about 20% and about 30%, and in a specific embodiment the enhancement is about 30%. T cell activation can be assessed, for example, with respect to particular T cell subpopulations, typically CD4⁺ or CD8⁺ T cells.

Suitable in vitro T cell activation assays, for assessing characteristics of such anti-pG6b antibodies, include T cell proliferation assays well-known in the art, including T cell proliferation assays as further described herein. Agonistic anti-CD3 and anti-CD28 antibodies, for stimulating TcR and CD28 signaling pathways, are also well-known in the art and commercially available, and include, for example, anti-CD3 mAb (555329) and anti-CD28 mAb (555725) from BD Biosciences. Exemplary T cell proliferation assays for anti-pG6b antibody characteristics are further described in Example 5, infra.

Anti-pG6b antibodies having the above-described in vitro biological properties can be readily obtained, for example, by using a polypeptide comprising the extracellular domain of pG6b (e.g., the extracellular domain of human pG6b as set forth in SEQ ID NO:3) as an immunogen for the production of polyclonal or monoclonal antibodies (using methods for antibody production such as discussed herein) and then screening the antibodies generated in in vitro T cell activation assays such as, e.g., T cell proliferation assays discussed herein to identify those antibodies having the desired properties. An exemplary method for generating antibodies that specifically bind to the extracellular domain of human pG6b (SEQ ID NO:3) is further described in Example 3, infra. For assessing in vitro functional characteristics, assays can include covalent coupling of pG6b antibodies to beads (e.g., tosylactivated beads, such as commercially available from Dynal Biotech ASA (Oslo, Norway)), together with anti-CD3 mAb, to screen for antibodies that have inhibitory effects against anti-CD3 and/or anti-CD3/anti-CD28-induced T cell activation. Alternatively, soluble anti-pB6b antibodies can be tested for inhibitory effects against anti-CD3-induced T cell activation in the absence of CD28 co-stimulation, and/or for enhancement of anti-CD3/anti-CD28-induced T cell activation. Exemplary in vitro assays for assessing inhibitory and/or stimulatory effects of anti-pG6b antibodies, and which can be used to readily identify anti-pG6b antibodies having the desired characteristics, are further described in Example 5, infra.

Hybridomas expressing antibodies were produced and screened using methods similar to those described above to identify monoclonal anti-pG6b antibodies that have T-cell inhibitory activity when immobilized to tosylactivated beads in vitro. Exemplary hybridomas expressing these monoclonal antibodies were deposited with the American Type Tissue Culture Collection (ATCC; Manassas, Va.) patent depository as original deposits under the Budapest Treaty and were given the following ATCC Accession Nos.: clone 337.1.4 (ATCC Patent Deposit Designation PTA-8730); clone 337.3.3 (ATCC Patent Deposit Designation PTA-8731); clone 337.6.5.1 (ATCC Patent Deposit Designation PTA-8728); clone 337.8.35.3 (ATCC Patent Deposit Designation PTA-8729), all deposited on Oct. 24, 2007.

Accordingly, in some embodiments, the present invention provides an anti-pG6b monoclonal antibody that competes for binding to the extracellular domain of human pG6b (SEQ ID NO:3) with at least one of the antibodies produced by the hybridomas of clone designation numbers 337.1.4, 337.3.3, 337.6.5.1, and 337.8.35.3. In certain embodiments, the monoclonal antibody is the antibody produced by one of these hybridomas. In other embodiments, the antibody is an antigen-binding fragment and/or genetically engineered variant of such an antibody. In some variations, the anti-pG6b antibody includes at least one, at least two, or at least three complementarity determining region(s) (CDRs) of an antibody produced by the hybridoma selected from clone designation numbers 337.1.4, 337.3.3, 337.6.5.1, and 337.8.35.3. For example, the anti-pG6b antibody can include (i) a heavy chain variable region having CDRs H1, H2, and H3 of the antibody produced by the hybridoma selected from clone designation numbers 337.1.4, 337.3.3, 337.6.5.1, and 337.8.35.3; and/or (ii) a light chain variable region having CDRs L1, L2, and L3 of the antibody produced by the hybridoma selected from clone designation numbers 337.1.4, 337.3.3, 337.6.5.1, and 337.8.35.3. In more particular variations, the anti-pG6b antibody includes the heavy chain variable region, light chain variable region, or both the heavy and light chain variable regions, of the antibody produced by the hybridoma selected from clone designation numbers 337.1.4, 337.3.3, 337.6.5.1, and 337.8.35.3. In specific embodiments, the anti-pG6b antibody is a humanized antibody comprising (i) a heavy chain variable region having CDRs H1, H2, and H3 of the antibody produced by the hybridoma selected from clone designation numbers 337.1.4, 337.3.3, 337.6.5.1, and 337.8.35.3; and (ii) a light chain variable region having CDRs L1, L2, and L3 of the antibody produced by the hybridoma selected from clone designation numbers 337.1.4, 337.3.3, 337.6.5.1, and 337.8.35.3. In some variations, the anti-pG6b antibody is a single-chain antibody such as, for example, as single-chain Fv (scFv).

Anti-pG6b antibodies or antibody fragments of the present invention can be PEGylated using methods in the art and described herein.

Moreover, polyclonal anti-idiotype antibodies can be prepared by immunizing animals with anti-pG6b antibodies or antibody fragments, using standard techniques. See, e.g., Green et al., “Production of Polyclonal Antisera,” in Methods In Molecular Biology: Immunochemical Protocols, Manson (ed.), pages 1-12 (Humana Press 1992). Also, see Coligan at pages 2.4.1-2.4.7. Alternatively, monoclonal anti-idiotype antibodies can be prepared using anti-pG6b antibodies or antibody fragments as immunogens with the techniques, described above. As another alternative, humanized anti-idiotype antibodies or subhuman primate anti-idiotype antibodies can be prepared using the above-described techniques. Methods for producing anti-idiotype antibodies are described, for example, by Irie, U.S. Pat. No. 5,208,146, Greene, et. al., U.S. Pat. No. 5,637,677, and Varthakavi and Minocha, J. Gen. Virol. 77:1875, 1996.

An anti-pG6b antibody can be conjugated with a detectable label to form an anti-pG6b immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the present invention are ³H, ¹²⁵I, ¹³¹I, ³⁵S and ¹⁴C.

Anti-pG6b immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-pG6b immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-pG6b immunoconjugates of the present invention. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-pG6b immunoconjugates can be detectably labeled by linking an anti-pG6b antibody component to an enzyme. When the anti-pG6b-enzyme conjugate is incubated in the presence of the appropriate substrate, the enzyme moiety reacts with the substrate to produce a chemical moiety which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels which can be employed in accordance with the present invention. The binding of marker moieties to anti-pG6b antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Int'l J. Cancer 46:1101 (1990), Stein et al., Cancer Res. 50:1330 (1990), and Coligan, supra.

Moreover, the convenience and versatility of immunochemical detection can be enhanced by using anti-pG6b antibodies that have been conjugated with avidin, streptavidin, and biotin (see, e.g., Wilchek et al. (eds.), “Avidin-Biotin Technology,” Methods In Enzymology, Vol 184 (Academic Press 1990), and Bayer et al., “Immunochemical Applications of Avidin-Biotin Technology,” in Methods In Molecular Biology, Vol 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies: Production, Engineering, and Clinical Application, Ritter and Ladyman (eds.), pages 180-208, (Cambridge University Press, 1995), Perry, “The Role of Monoclonal Antibodies in the Advancement of Immunoassay Technology,” in Monoclonal Antibodies: Principles and Applications, Birch and Lennox (eds.), pages 107-120 (Wiley-Liss, Inc. 1995), and Diamandis, Immunoassay (Academic Press, Inc. 1996).

The present invention also contemplates kits for performing an immunological diagnostic assay for pG6b gene expression. Such kits comprise at least one container comprising an anti-pG6b antibody, or antibody fragment. A kit may also comprise a second container comprising one or more reagents capable of indicating the presence of pG6b antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that pG6b antibodies or antibody fragments are used to detect pG6b protein. For example, written instructions may state that the enclosed antibody or antibody fragment can be used to detect pG6b. The written material can be applied directly to a container, or the written material can be provided in the form of a packaging insert.

### 9. Use of Anti-pG6b Antibodies to Agonize or Antagonize pG6b Binding to its Counter-Receptor

Alternative techniques for generating or selecting antibodies useful herein include in vitro exposure of lymphocytes to soluble pG6b receptor polypeptides or fragments thereof, such as antigenic epitopes, and selection of antibody display libraries in phage or similar vectors (for instance, through use of immobilized or labeled soluble pG6b receptor polypeptides or fragments thereof, such as antigenic epitopes). Genes encoding polypeptides having potential binding domains such as soluble pG6b receptor polypeptides or fragments thereof, such as antigenic epitopes can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides that interact with a known target that can be a protein or polypeptide, such as a counter-receptor or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the soluble pG6b receptor polypeptides or fragments thereof, such as antigenic epitope polypeptide sequences disclosed herein to identify proteins which bind to pG6b-comprising receptor polypeptides. These “binding polypeptides,” which interact with soluble pG6b-comprising receptor polypeptides, can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and for agonizing and/or neutralizing activity, e.g., for binding, blocking, inhibiting, reducing, antagonizing or neutralizing interaction between pG6b and its counter-receptor. The binding polypeptides can also be used for diagnostic assays for determining circulating levels of soluble pG6b-comprising receptor polypeptides; for detecting or quantitating soluble or non-soluble pG6b-comprising receptors as marker of underlying pathology or disease. These binding polypeptides can also act as “antagonists” to block or inhibit soluble or membrane-bound pG6b monomeric receptor or pG6b homodimeric, heterodimeric or multimeric polypeptide binding (e.g. to counter-receptor) and signal transduction in vitro and in vivo. Again, these binding polypeptides serve as anti-pG6b monomeric receptor or anti-pG6b homodimeric, heterodimeric or multimeric polypeptides and are useful for inhibiting pG6b activity, as well as pG6b counter-receptor activity or protein-binding. Antibodies raised to the natural receptor complexes of the present invention, and pG6b-epitope-binding antibodies, and anti-pG6b neutralizing monoclonal antibodies may be preferred embodiments, as they may act more specifically against the pG6b and can inhibit its binding to its counter-receptor. Moreover, the agonistic, antagonistic and binding activity of the antibodies of the present invention can be assayed in a pG6b proliferation, signal trap, luciferase or binding assays in the presence of its counter-receptor or any other B7 family receptor, and pG6b-comprising soluble receptors, and other biological or biochemical assays described herein.

Antibodies to pG6b receptor polypeptides (e.g., antibodies to SEQ ID NO:2) or fragments thereof, such as antigenic epitopes may be used for inhibiting the inflammatory effects of pG6b in vivo, for therapeutic use against rheumatoid arthritis, psoriasis, atopic dermatitis, inflammatory skin conditions, endotoxemia, arthritis, asthma, IBD, colitis, psoriatic arthritis, multiple sclerosis, or other inflammatory conditions; tagging cells that express pG6b receptors; for isolating soluble pG6b-comprising receptor polypeptides by affinity purification; for diagnostic assays for determining circulating levels of soluble pG6b-comprising receptor polypeptides; for detecting or quantitating soluble pG6b-comprising receptors as marker of underlying pathology or disease; in analytical methods employing FACS; for screening expression libraries; for generating anti-idiotypic antibodies that can act as pG6b agonists; and as neutralizing antibodies or as antagonists to bind, block, inhibit, reduce, or antagonize pG6b receptor function, or to bind, block, inhibit, reduce, antagonize or neutralize pG6b activity in vitro, ex vivo, and in vivo. Suitable direct tags or labels include radionuclides, enzymes, substrates, cofactors, biotin, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to soluble pG6b-comprising receptor polypeptides, or fragments thereof may be used in vitro to detect denatured or non-denatured pG6b-comprising receptor polypeptides or fragments thereof in assays, for example, Western Blots or other assays known in the art.

Antibodies to soluble pG6b receptor or soluble pG6b homodimeric, heterodimeric or multimeric receptor polypeptides are useful for tagging cells that express the corresponding receptors and assaying their expression levels, for affinity purification, within diagnostic assays for determining circulating levels of receptor polypeptides, analytical methods employing fluorescence-activated cell sorting. Moreover, divalent antibodies, and anti-idiotypic antibodies may be used as agonists to mimic the effect of pG6b.

Antibodies herein can also be directly or indirectly conjugated to drugs, toxins, radionuclides and the like, and these conjugates used for in vivo diagnostic or therapeutic applications. For instance, antibodies or binding polypeptides which recognize soluble pG6b receptor or soluble pG6b homodimeric, heterodimeric or multimeric receptor polypeptides can be used to identify or treat tissues or organs that express a corresponding anti-complementary molecule (i.e., a pG6b-comprising soluble or membrane-bound receptor). More specifically, antibodies to soluble pG6b-comprising receptor polypeptides, or bioactive fragments or portions thereof, can be coupled to detectable or cytotoxic molecules and delivered to a mammal having cells, tissues or organs that express the pG6b-comprising receptor such as pG6b-expressing cancers.

Suitable detectable molecules may be directly or indirectly attached to polypeptides that bind pG6b-comprising receptor polypeptides, such as “binding polypeptides,” (including binding peptides disclosed above), antibodies, or bioactive fragments or portions thereof. Suitable detectable molecules include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Binding polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the binding polypeptide or antibody portion. For these purposes, biotin/streptavidin is an exemplary complementary/anticomplementary pair.

In another embodiment, binding polypeptide-toxin fusion proteins or antibody-toxin fusion proteins can be used for targeted cell or tissue inhibition or ablation (for instance, to treat cancer cells or tissues). Alternatively, if the binding polypeptide has multiple functional domains (i.e., an activation domain or a counter-receptor binding domain, plus a targeting domain), a fusion protein including only the targeting domain may be suitable for directing a detectable molecule, a cytotoxic molecule or a complementary molecule to a cell or tissue type of interest. In instances where the fusion protein including only a single domain includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/cytotoxic molecule conjugates.

Alternatively, pG6b receptor binding polypeptides or antibody fusion proteins described herein can be used for enhancing in vivo killing of target tissues by directly stimulating a pG6b receptor-modulated apoptotic pathway, resulting in cell death of hyperproliferative cells expressing pG6b-comprising receptors.

### 10. Therapeutic Uses of Polypeptides Having pG6b Activity or Antibodies to pG6b

Amino acid sequences having soluble pG6b activity can be used to modulate the immune system by binding pG6b counter-receptors, and thus, preventing the binding of pG6b counter-receptor with endogenous pG6b receptor. pG6b antagonists, such as anti-pG6b antibodies, can also be used to modulate the immune system by inhibiting the binding of pG6b counter-receptor with the endogenous pG6b receptor. Accordingly, the present invention includes the use of proteins, polypeptides, and peptides having pG6b activity (such as soluble pG6b polypeptides, pG6b polypeptide fragments, pG6b analogs (e.g., anti-pG6b anti-idiotype antibodies), and pG6b fusion proteins) to a subject which lacks an adequate amount of this polypeptide, or which produces an excess of pG6b counter-receptor. pG6b antagonists (e.g., anti-pG6b antibodies) can be also used to treat a subject which produces an excess of either pG6b counter-receptor or pG6b. Suitable subjects include mammals, such as humans.

For example, in certain aspects, pG6b antagonists are useful for the treatment of cancer. Without intending to be bound to any particular mechanism of action, it is contemplated that as a negative regulator of T lymphocyte activity, pG6b acts to inhibit one or more stimulatory signals necessary to fully activate T cells, thereby potentially contributing to poor immunogenicity of certain tumors. Accordingly, blockade of an inhibitory pG6b-induced signal, such as with an antagonistic anti-pG6b antibody, can be useful for increasing a host immune response against tumor cells in the treatment of cancer. Such treatment of cancer with blockade of another negative regulator of T cell activity, CTLA-4 (also a CD28 family member), has been previously shown (see, e.g., Leach et al., Science 271:1734-1736, 1996), and the use of blocking anti-CTLA-4 mAbs for treatment of cancer is currently undergoing clinal trials.

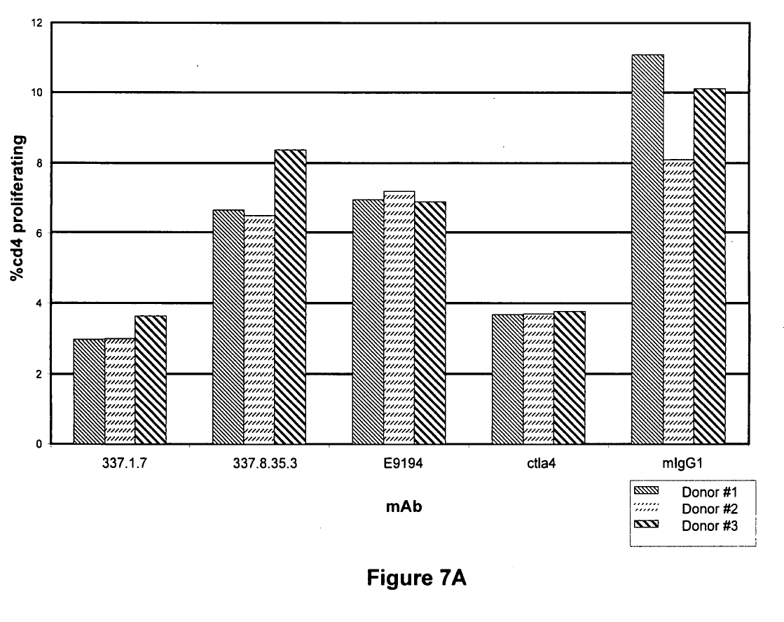

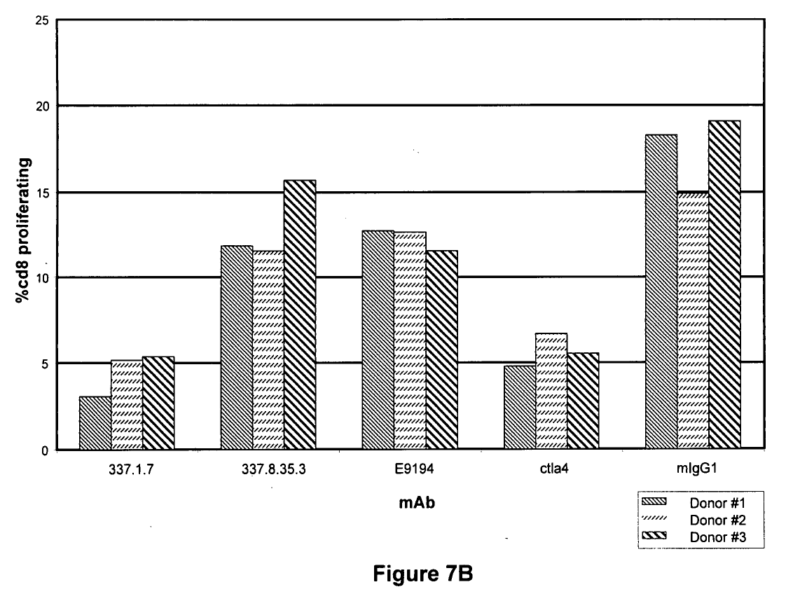

Accordingly, in specific aspects, antibodies useful for providing a blocking effect in vivo include anti-pG6b antibodies of the present invention that show inhibitory activity against T cells when covalently coupled to beads in vitro. In this respect, the present inventors found that certain anti-pG6b monoclonal antibodies exhibited in vitro inhibitory activity against T cells when covalently coupled to beads, similar to, and in some cases approximately equal to, inhibitory activity observed for anti-CTLA-4 mAb. (See Example 5 and FIGS. 7, 8, and 9.) Exemplary antibodies exhibiting such activity include antibodies produced by the hybridomas of clone designation numbers 337.1.4, 337.3.3, 337.6.5.1, and 337.8.35.3. It has been shown that anti-CTLA-4 antibodies having such inhibitory activity against anti-CD3 and anti-CD3/anti-CD28-induced T cell activation when such CTLA-4 mAb is presented under cross-linking conditions (such as, e.g., immobilization on beads) also enhance T cell activation via blockade under certain conditions where the anti-CTLA-4 mAb is presented in a non-cross-linked form. (See, e.g., Krummel and Allison, J. Exp. Med. 182:459-465, 1995.) Thus, the in vitro results observed with respect to covalently coupled anti-pG6b antibodies are consistent with a negative regulatory role for pG6b in T cell activation, and further indicate that such antibodies can act to block inhibitory pG6b activity in vivo to so as to enhance positive T cell co-stimulation, such as to increase a host immune response against cancer.

Other antibodies of the invention are also useful for increasing host immune responses, such as against cancer, and include, for example, anti-pG6b antibodies exhibiting enhancement of anti-CD3/anti-CD28-induced T cell activation when presented to T cells in soluble form.

In other aspects, agonistic pG6b polypeptides and anti-pG6b antibodies are useful for down-regulating an immune response by inducing pG6b-mediated inhibition of T cell activation. Accordingly, in certain embodiments, such polypeptides and antibodies are useful in the treatment of autoimmune or inflammatory disease, including, for example, psoriasis, atopic dermatitis, inflammatory skin conditions, psoriatic arthritis, arthritis, endotoxemia, asthma, inflammatory bowel disease (IBD), colitis, and other inflammatory conditions disclosed herein.

Accordingly, in some variations, the present invention is in particular a method for treating psoriasis by administering agents are in vivo agonists of pG6b. In some embodiment, the agonists of pG6b are anti-pG6b antibodies that bind pG6b so as to mimic or augment the interaction of pG6b and a pG6b counter-receptor. Such agonists can be administered alone or in combination with other established therapies such as lubricants, keratolytics, topical corticosteroids, topical vitamin D derivatives, anthralin, systemic antimetabolites such as methotrexate, psoralen-ultraviolet-light therapy (PUVA), etretinate, isotretinoin, cyclosporine, and the topical vitamin D3 derivative calcipotriol. Moreover, such agonists can be administered to individual subcutaneously, intravenously, or transdermally using a cream or transdermal patch that contains the antagonist. If administered subcutaneously, the agonist can be injected into one or more psoriatic plaques. If administered transdermally, the agonists can be administered directly on the plaques using a cream, ointment, salve, or solution containing the agonist.

Agonists to pG6b can also be administered to a person who has asthma, bronchitis or cystic fibrosis or other inflammatory lung disease to treat the disease. The agonists can be administered by any suitable method including intravenous, subcutaneous, bronchial lavage, and the use of inhalant containing the antagonist.

Thus, particular embodiments of the present invention are directed toward use of anti-pG6b antibodies as pG6b agonists in inflammatory and immune diseases or conditions such as psoriasis, psoriatic arthritis, atopic dermatitis, inflammatory skin conditions, rheumatoid arthritis, inflammatory bowel disease (IBD), Crohn's Disease, diverticulosis, asthma, pancreatitis, type I diabetes (IDDM), pancreatic cancer, pancreatitis, Graves Disease, autoimmune disease, sepsis, organ or bone marrow transplant; inflammation due to endotoxemia, trauma, surgery or infection; splenomegaly; graft versus host disease; and where inhibition of inflammation, immune suppression, reduction of proliferation of hematopoietic, immune, inflammatory or lymphoid cells, macrophages, T-cells (including Th1 and Th2 cells), suppression of immune response to a antigen, or other instances where enhancement of a pG6b-induced inhibitory signal is desired.

Moreover, antibodies or binding polypeptides that bind pG6b polypeptides described herein, and pG6b polypeptides themselves are useful to:

(1) Block, inhibit, reduce, antagonize or neutralize signaling via pG6b in the treatment of acute inflammation, inflammation as a result of trauma, tissue injury, surgery, sepsis or infection, and chronic inflammatory diseases such as asthma, inflammatory bowel disease (IBD), chronic colitis, splenomegaly, rheumatoid arthritis, recurrent acute inflammatory episodes (e.g., tuberculosis), and treatment of amyloidosis, and atherosclerosis, Castleman's Disease, asthma, and other diseases associated with the induction of acute-phase response.

(2) Block, inhibit, reduce, antagonize or neutralize signaling via pG6b in the treatment of autoimmune diseases such as IDDM, multiple sclerosis (MS), systemic Lupus erythematosus (SLE), myasthenia gravis, rheumatoid arthritis, and IBD to prevent or inhibit signaling in immune cells (e.g. lymphocytes, monocytes, leukocytes) via pG6b (Hughes C et al., J. Immunol. 153: 3319-3325, 1994). Alternatively antibodies, such as monoclonal antibodies (MAb) to pG6b, can also be used as an antagonist to deplete unwanted immune cells to treat autoimmune disease. Asthma, allergy and other atopic disease may be treated with an MAb against, for example, soluble pG6b soluble receptors to inhibit the immune response or to deplete offending cells. Blocking, inhibiting, reducing, or antagonizing signaling via pG6b, using the polypeptides and antibodies of the present invention, may also benefit diseases of the pancreas, kidney, pituitary and neuronal cells. IDDM, NIDDM, pancreatitis, and pancreatic carcinoma may benefit. pG6b may serve as a target for mAb therapy of cancer where an antagonizing MAb inhibits cancer growth and targets immune-mediated killing. (Holliger and Hoogenboom, Nature Biotech. 16: 1015-1016, 1998). Mabs to soluble pG6b may also be useful to treat nephropathies such as glomerulosclerosis, membranous neuropathy, amyloidosis (which also affects the kidney among other tissues), renal arteriosclerosis, glomerulonephritis of various origins, fibroproliferative diseases of the kidney, as well as kidney dysfunction associated with SLE, IDDM, type II diabetes (NIDDM), renal tumors and other diseases.

(3) Agonize, enhance, increase or initiate signaling via pG6b in the treatment of autoimmune diseases such as IDDM, MS, SLE, myasthenia gravis, rheumatoid arthritis, and IBD. Anti-pG6b neutralizing and monoclonal antibodies may signal lymphocytes or other immune cells to differentiate, alter proliferation, or change production of cytokines or cell surface proteins that ameliorate autoimmunity. Specifically, modulation of a T-cell response may deviate an autoimmune response to ameliorate disease (Smith et al., J. Immunol. 160:4841-4849, 1998). Similarly, agonistic anti-pG6b monoclonal antibodies may be used to signal, deplete and deviate immune cells involved in rheumatoid arthritis, asthma, allergy and atopoic disease. Signaling via pG6b may also benefit diseases of the pancreas, kidney, pituitary and neuronal cells. IDDM, NIDDM, pancreatitis, and pancreatic carcinoma may benefit. pG6b may serve as a target for MAb therapy of pancreatic cancer where a signaling MAb inhibits cancer growth and targets immune-mediated killing (Tutt, A L et al., J Immunol. 161: 3175-3185, 1998). Similarly renal cell carcinoma may be treated with monoclonal antibodies to pG6b-comprising soluble receptors of the present invention.

Soluble pG6b polypeptides described herein can be used to bind, block, inhibit, reduce, antagonize or neutralize pG6b activity, either singly or together, in the treatment of autoimmune disease, atopic disease, NIDDM, pancreatitis and kidney dysfunction as described above. A soluble form of pG6b may be used to promote an antibody response mediated by Th cells and/or to promote the production of IL-4 or other cytokines by lymphocytes or other immune cells.

Moreover, inflammation is a protective response by an organism to fend off an invading agent. Inflammation is a cascading event that involves many cellular and humoral mediators. On one hand, suppression of inflammatory responses can leave a host immunocompromised; however, if left unchecked, inflammation can lead to serious complications including chronic inflammatory diseases (e.g., psoriasis, arthritis, rheumatoid arthritis, multiple sclerosis, inflammatory bowel disease and the like), septic shock and multiple organ failure. Importantly, these diverse disease states share common inflammatory mediators. The collective diseases that are characterized by inflammation have a large impact on human morbidity and mortality. Therefore it is clear that molecules that are intimately involved in the costimulation and/or inhibition of immune responses, such as pG6b, its counter-receptor, and anti-pG6b antibodies, could have crucial therapeutic potential for a vast number of human and animal diseases, from asthma and allergy to autoimmunity and septic shock.

1. Arthritis

Arthritis, including osteoarthritis, rheumatoid arthritis, arthritic joints as a result of injury, and the like, are common inflammatory conditions which would benefit from the therapeutic use of anti-inflammatory proteins, such as pG6b polypeptides of the present invention. For example, rheumatoid arthritis (RA) is a systemic disease that affects the entire body and is one of the most common forms of arthritis. It is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

Rheumatoid arthritis (RA) is an immune-mediated disease particularly characterized by inflammation and subsequent tissue damage leading to severe disability and increased mortality. A variety of cytokines are produced locally in the rheumatoid joints. Numerous studies have demonstrated that IL-1 and TNF-alpha, two prototypic pro-inflammatory cytokines, play an important role in the mechanisms involved in synovial inflammation and in progressive joint destruction. Indeed, the administration of TNF-alpha and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2(2):135-149, 2002). One of those mediators could be pG6b or its counter-receptor, and as such a molecule that binds or inhibits pG6b activity, such as pG6b polypeptides, or anti-pG6b antibodies or binding partners, could serve as a valuable therapeutic to reduce inflammation in rheumatoid arthritis, and other arthritic diseases.

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol 89:9784-788, 1992; Myers et al., Life Sci. 61:1861-78, 1997; and Wang et al., Immunol. 92:8955-959, 1995).

2. Endotoxemia

Endotoxemia is a severe condition commonly resulting from infectious agents such as bacteria and other infectious disease agents, sepsis, toxic shock syndrome, or in immunocompromised patients subjected to opportunistic infections, and the like. Therapeutically useful of anti-inflammatory proteins, such as pG6b polypeptides and antibodies of the present invention, could aid in preventing and treating endotoxemia in humans and animals. pG6b polypeptides, anti-IL22RA antibodies, or anti IL-22 antibodies or binding partners, could serve as a valuable therapeutic to reduce inflammation and pathological effects in endotoxemia.

Lipopolysaccharide (LPS) induced endotoxemia engages many of the proinflammatory mediators that produce pathological effects in the infectious diseases and LPS induced endotoxemia in rodents is a widely used and acceptable model for studying the pharmacological effects of potential pro-inflammatory or immunomodulating agents. LPS, produced in gram-negative bacteria, is a major causative agent in the pathogenesis of septic shock (Glausner et al., Lancet 338:732, 1991). A shock-like state can indeed be induced experimentally by a single injection of LPS into animals. Molecules produced by cells responding to LPS can target pathogens directly or indirectly. Although these biological responses protect the host against invading pathogens, they may also cause harm. Thus, massive stimulation of innate immunity, occurring as a result of severe Gram-negative bacterial infection, leads to excess production of cytokines and other molecules, and the development of a fatal syndrome, septic shock syndrome, which is characterized by fever, hypotension, disseminated intravascular coagulation, and multiple organ failure (Dumitru et al. Cell 103:1071-1083, 2000).

These toxic effects of LPS are mostly related to macrophage activation leading to the release of multiple inflammatory mediators. Among these mediators, TNF appears to play a crucial role, as indicated by the prevention of LPS toxicity by the administration of neutralizing anti-TNF antibodies (Beutler et al., Science 229:869, 1985). It is well established that lug injection of E. coli LPS into a C57B1/6 mouse will result in significant increases in circulating IL-6, TNF-alpha, IL-1, and acute phase proteins (for example, SAA) approximately 2 hours post injection. The toxicity of LPS appears to be mediated by these cytokines as passive immunization against these mediators can result in decreased mortality (Beutler et al., Science 229:869, 1985). The potential immunointervention strategies for the prevention and/or treatment of septic shock include anti-TNF mAb, IL-1 receptor antagonist, LIF, IL-10, and G-CSF.

The administration of anti-pG6b antibodies or other pG6b soluble and fusion proteins to these LPS-induced model can be used to evaluate the use of pG6b to ameliorate symptoms and alter the course of LPS-induced disease.

3. Inflammatory Bowel Disease (IBD)

In the United States approximately 500,000 people suffer from Inflammatory Bowel Disease (IBD) which can affect either colon and rectum (Ulcerative colitis) or both, small and large intestine (Crohn's Disease). The pathogenesis of these diseases is unclear, but they involve chronic inflammation of the affected tissues. pG6b polypeptides, anti-pG6b antibodies, or binding partners, could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss. Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids immunosuppressives (eg. azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. The most widely used model is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanies by the clinical picture of progressive weight loss (wasting), bloody diarrhea, rectal prolapse, and large bowel wall thickening (Neurath et al. Intern. Rev. Immunol 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of anti-pG6b antibodies or other pG6b soluble and fusion proteins to these TNBS or DSS models can be used to evaluate the use of pG6b to ameliorate symptoms and alter the course of gastrointestinal disease. Moreover, the results showing inhibition of T cell signaling by pG6b provide proof of concept that other pG6b antagonists, such as pG6b or antibodies thereto, can also be used to ameliorate symptoms in the colitis/IBD models and alter the course of disease.

4. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. pG6b polypeptides, anti-pG6b antibodies, or anti IL-22 and anti pG6b antibodies or binding partners, could serve as a valuable therapeutic to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

Moreover, anti-pG6b antibodies and pG6b soluble receptors of the present invention can be used in the prevention and therapy against weight loss associated with a number of inflammatory diseases described herein, as well as for cancer (e.g., chemotherapy and cachexia), and infectious diseases. For example, severe weight loss is a key marker associated with models for septicemia, MS, RA, and tumor models. In addition, weight loss is a key parameter for many human diseases including cancer, infectious disease and inflammatory disease. Anti-pG6b antibodies and pG6b antagonists such as the soluble pG6b receptors and antibodies thereto of the present invention, can be tested for their ability to prevent and treat weight loss in mice injected with pG6b andenovires described herein. Methods of determining a prophylactic or therapeutic regimen for such pG6b antagonists is known in the art and can be determined using the methods described herein.

pG6b soluble receptor polypeptides and antibodies thereto may also be used within diagnostic systems for the detection of circulating levels of pG6b or pG6b counter-receptor, and in the detection of pG6b associated with acute phase inflammatory response. Within a related embodiment, antibodies or other agents that specifically bind to pG6b soluble receptors of the present invention can be used to detect circulating receptor polypeptides; conversely, pG6b soluble receptors themselves can be used to detect circulating or locally-acting pG6b polypeptides. Elevated or depressed levels of pG6b counter-receptor or pG6b polypeptides may be indicative of pathological conditions, including inflammation or cancer. Moreover, detection of acute phase proteins or molecules such as pG6b can be indicative of a chronic inflammatory condition in certain disease states (e.g., psoriasis, rheumatoid arthritis, colitis, IBD). Detection of such conditions serves to aid in disease diagnosis as well as help a physician in choosing proper therapy.

In addition to other disease models described herein, the activity of anti-pG6b antibodies on inflammatory tissue derived from human psoriatic lesions can be measured in vivo using a severe combined immune deficient (SCID) mouse model. Several mouse models have been developed in which human cells are implanted into immunodeficient mice (collectively referred to as xenograft models); see, for example, Cattan A R, Douglas E, Leuk. Res. 18:513-22, 1994 and Flavell, D J, Hematological Oncology 14:67-82, 1996. As an in vivo xenograft model for psoriasis, human psoriatic skin tissue is implanted into the SCID mouse model, and challenged with an appropriate antagonist. Moreover, other psoriasis animal models in the art may be used to evaluate pG6b antagonists, such as human psoriatic skin grafts implanted into AGR129 mouse model, and challenged with an appropriate antagonist (see, e.g., Boyman, O. et al., J. Exp. Med. Online publication #20031482, 2004, incorporated herein by reference). Anti-pG6b antibodies that bind, block, inhibit, reduce, antagonize or neutralize the activity of pG6b are preferred antagonists, however, anti-pG6b antibodies (alone or in combination with other B7 antagonists), soluble pG6b, as well as other pG6b antagonists can be used in this model. Similarly, tissues or cells derived from human colitis, IBD, arthritis, or other inflammatory lesions can be used in the SCID model to assess the anti-inflammatory properties of the pG6b antagonists described herein.

Therapies designed to abolish, retard, or reduce inflammation using anti-pG6b antibodies or its derivatives, agonists, conjugates or variants can be tested by administration of anti-pG6b antibodies or soluble pG6b compounds to SCID mice bearing human inflammatory tissue (e.g., psoriatic lesions and the like), or other models described herein. Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. See, e.g., Zeigler et al., Lab Invest 81:1253, 2001; Zollner et al., J. Clin. Invest. 109:671, 2002; Yamanaka et al., Microbiol Immunol 45:507, 2001; Raychaudhuri et al. Br. J. Dermatol 144:931, 2001; Boehncke et al., Arch. Dermatol Res. 291:104, 1999; Boehncke et al., J. Invest. Dermatol. 116:596, 2001; Nickoloff et al., Am. J. Pathol 146:580, 1995; Boehncke et al., J. Cutan. Pathol 24:1, 1997; Sugai et al., J. Dermatol. Sci. 17:85, 1998; and Villadsen et al., J. Clin. Invest. 112:1571, 2003. Inflammation may also be monitored over time using well-known methods such as flow cytometry (or PCR) to quantitate the number of inflammatory or lesional cells present in a sample, score (weight loss, diarrhea, rectal bleeding, colon length) for IBD, paw disease score and inflammation score for CIA R A model. For example, therapeutic strategies appropriate for testing in such a model include direct treatment using anti-pG6b antibodies, other pG6b agonists (singly or together with other B7 antagonists), or related conjugates or agonists based on mimicking or augmenting the interaction pG6b with a pG6b counter-receptor, or for cell-based therapies utilizing anti-pG6b antibodies or its derivatives, agonists, conjugates or variants.

Moreover, psoriasis is a chronic inflammatory skin disease that is associated with hyperplastic epidermal keratinocytes and infiltrating mononuclear cells, including CD4⁺ memory T cells, neutrophils and macrophages (Christophers, Int. Arch. Allergy Immunol. 110:199, 1996). It is currently believed that environmental antigens play a significant role in initiating and contributing to the pathology of the disease. However, it is the loss of tolerance to self-antigens that is thought to mediate the pathology of psoriasis. Dendritic cells and CD4⁺ T cells are thought to play an important role in antigen presentation and recognition that mediate the immune response leading to the pathology. We have recently developed a model of psoriasis based on the CD4⁺ CD45RB transfer model (Davenport et al., Internat. Immunopharmacol 2:653-672). Anti-pG6b antibodies of the present invention, or soluble pG6b, are administered to the mice. Inhibition of disease scores (skin lesions, inflammatory cytokines) indicates the effectiveness of pG6b agonists in psoriasis, e.g., anti-pG6b antibodies.

5. Atopic Dermatitis.

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western countries suffers from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents. The soluble pG6b polypeptides and anti-pG6b antibodies of the present invention can be used in the treatment of specific human diseases such as atopic dermatitis, inflammatory skin conditions, and other inflammatory conditions disclosed herein.

For pharmaceutical use, the soluble pG6b or anti-pG6b antibodies of the present invention are formulated for parenteral, particularly intravenous or subcutaneous, delivery according to conventional methods. Intravenous administration will be by bolus injection, controlled release, e.g, using mini-pumps or other appropriate technology, or by infusion over a typical period of one to several hours. In general, pharmaceutical formulations will include a hematopoietic protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. When utilizing such a combination therapy, the cytokines may be combined in a single formulation or may be administered in separate formulations. Methods of formulation are well known in the art and are disclosed, for example, in Remington 's Pharmaceutical Sciences, Gennaro, ed., Mack Publishing Co., Easton Pa., 1990, which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins will commonly be administered over a period of up to 28 days following chemotherapy or bone-marrow transplant or until a platelet count of >20,000/mm³, preferably >50,000/mm³, is achieved. More commonly, the proteins will be administered over one week or less, often over a period of one to three days. In general, a therapeutically effective amount of soluble pG6b or anti-pG6b antibodies of the present invention is an amount sufficient to produce a clinically significant increase in the proliferation and/or differentiation of lymphoid or myeloid progenitor cells, which will be manifested as an increase in circulating levels of mature cells (e.g. platelets or neutrophils). Treatment of platelet disorders will thus be continued until a platelet count of at least 20,000/mm³, preferably 50,000/mm³, is reached. The soluble pG6b or anti-pG6b antibodies of the present invention can also be administered in combination with other cytokines such as IL-3, -6 and -11; stem cell factor; erythropoietin; G-CSF and GM-CSF. Within regimens of combination therapy, daily doses of other cytokines will in general be: EPO, 150 U/kg; GM-CSF, 5-15 lg/kg; IL-3, 1-5 lg/kg; and G-CSF, 1-25 lg/kg. Combination therapy with EPO, for example, is indicated in anemic patients with low EPO levels.

Generally, the dosage of administered soluble pG6b (or pG6b analog or fusion protein) or anti-pG6b antibodies will vary depending upon such factors as the patient's age, weight, height, sex, general medical condition and previous medical history. Typically, it is desirable to provide the recipient with a dosage of soluble pG6b or anti-pG6b antibodies which is in the range of from about 1 pg/kg to 10 mg/kg (amount of agent/body weight of patient), although a lower or higher dosage also may be administered as circumstances dictate.

Administration of soluble pG6b or anti-pG6b antibodies to a subject can be intravenous, intraarterial, intraperitoneal, intramuscular, subcutaneous, intrapleural, intrathecal, by perfusion through a regional catheter, or by direct intralesional injection. When administering therapeutic proteins by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, for example, Hinchcliffe and Illum, Adv. Drug Deliv. Rev. 35:199 (1999)). Dry or liquid particles comprising pG6b can be prepared and inhaled with the aid of dry-powder dispersers, liquid aerosol generators, or nebulizers (e.g., Pettit and Gombotz, TIBTECH 16:343, 1998; Patton et al., Adv. Drug Deliv. Rev. 35:235, 1999). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al, Science 269:850, 1995). Transdermal delivery using electroporation provides another means to administer a molecule having pG6b binding activity (Potts et al., Pharm. Biotechnol 10:213, 1997).

A pharmaceutical composition comprising a soluble pG6b or anti-pG6b antibody can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, e.g., Gennaro (ed.), Remington's Pharmaceutical Sciences, 19th Edition (Mack Publishing Company 1995).

For purposes of therapy, soluble pG6b or anti-pG6b antibody molecules and a pharmaceutically acceptable carrier are administered to a patient in a therapeutically effective amount. A combination of a therapeutic molecule of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response.

A pharmaceutical composition comprising pG6b (or pG6b analog or fusion protein) or anti-pG6b antibody can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol 10:239, 1997; Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)).

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see generally Bakker-Woudenberg et al., Eur. J. Clin. Microbiol. Infect. Dis. 12 (Suppl. 1):S61, 1993; Kim, Drugs 46:618, 1993; and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, e.g., Machy et al., Liposomes In Cell Biology And Pharmacology (John Libbey 1987), and Ostro et al., American J. Hosp. Pharm. 46:1576 (1989)). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368, 1985). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428, 1984). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133, 1991; Allen et al., Biochim. Biophys. Acta 1150:9, 1993).

Liposomes can also be prepared to target particular cells or organs by varying phospholipid composition or by inserting receptors or counter-receptors into the liposomes. For example, liposomes, prepared with a high content of a nonionic surfactant, have been used to target the liver (Hayakawa et al., Japanese Patent 04-244,018; Kato et al., Biol. Pharm. Bull. 16:960, 1993). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881, 1997).

Alternatively, various targeting counter-receptors can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst. 14:287, 1997; Murahashi et al., Biol. Pharm. Bull. 20:259, 1997). Similarly, Wu et al. (Hepatology 27:772, 1998) have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull 20:259, 1997). Polyaconitylated human serum albumin liposomes provide another approach for targeting liposomes to liver cells (Kamps et al., Proc. Nat'l Acad. Sci. USA 94:11681, 1997). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a counter-receptor expressed by the target cell (Harasym et al., Adv. Drug Deliv. Rev. 32:99, 1998). After plasma elimination of free antibody, streptavidin-conjugated liposomes are administered. In another approach, targeting antibodies are directly attached to liposomes (Harasym et al., Adv. Drug Deliv. Rev. 32:99, 1998).

Polypeptides and antibodies can be encapsulated within liposomes using standard techniques of protein microencapsulation (see, e.g., Anderson et al., Infect. Immun. 31:1099, 1981; Anderson et al., Cancer Res. 50:1853, 1990; Cohen et al., Biochim. Biophys. Acta 1063:95, 1991; Alving et al. “Preparation and Use of Liposomes in Immunological Studies,” in Liposome Technology, 2nd Edition, Vol. III, Gregoriadis (ed.), page 317 (CRC Press 1993); Wassef et al., Meth. Enzymol. 149:124, 1987). As noted above, therapeutically useful liposomes may contain a variety of components. For example, liposomes may comprise lipid derivatives of poly(ethylene glycol) (Allen et al., Biochim. Biophys. Acta 1150:9, 1993).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332, 1995; Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161, 1998; Putney and Burke, Nature Biotechnology 16:153, 1998; Putney, Curr. Opin. Chem. Biol. 2:548, 1998). Polyethylene glycol (PEG)-coated nanospheres can also provide carriers for intravenous administration of therapeutic proteins (see, e.g., Gref et al., Pharm. Biotechnol 10:167, 1997).

The present invention also contemplates chemically modified polypeptides having binding pG6b activity such as pG6b monomeric, homodimeric, heterodimeric or multimeric soluble receptors, and pG6b antagonists, for example anti-pG6b antibodies or binding polypeptides, or neutralizing anti-pG6b antibodies, which a polypeptide is linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

As an illustration, pharmaceutical compositions may be supplied as a kit comprising a container that comprises a polypeptide with a pG6b extracellular domain, e.g., pG6b monomeric, homodimeric, heterodimeric or multimeric soluble receptors, or a pG6b antagonist (e.g., an antibody or antibody fragment that binds a pG6b polypeptide, or neutralizing anti-pG6b antibody). Therapeutic polypeptides can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise writ en information on indications and usage of the pharmaceutical composition. Moreover, such information may include a statement that the pG6b composition is contraindicated in patients with known hypersensitivity to pG6b.

A pharmaceutical composition comprising anti-pG6b antibodies or binding partners (or Anti-pG6b antibody fragments, antibody fusions, humanized antibodies and the like), or pG6b soluble receptor, can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants (Bremer et al., Pharm. Biotechnol 10:239, 1997; Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Deliver: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

Liposomes provide one means to deliver therapeutic polypeptides to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see, generally, Bakker-Woudenberg et al., Eur. J. Clin. Microbiol. Infect. Dis. 12 (Suppl. 1):S61, 1993; Kim, Drugs 46:618, 1993; and Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, for example, Machy et al., Liposomes In Cell Biology And Pharmacology (John Libbey 1987), and Ostro et al., American J. Hosp. Pharm. 46:1576, 1989). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368, 1985). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428, 1984). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133, 1991; Allen et al., Biochim. Biophys. Acta 1150:9, 1993).

Liposomes can also be prepared to target particular cells or organs by varying phospholipid composition or by inserting receptors or counter-receptors into the liposomes. For example, liposomes, prepared with a high content of a nonionic surfactant, have been used to target the liver (Hayakawa et al., Japanese Patent 04-244,018; Kato et al., Biol. Pharm. Bull. 16:960, 1993). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull 20:881, 1997).

Alternatively, various targeting counter-receptors can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst. 14:287, 1997; Murahashi et al., Biol. Pharm. Bull. 20:259, 1997). Similarly, Wu et al., Hepatology 27:772, 1998, have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull 20:259, 1997). Polyaconitylated human serum albumin liposomes provide another approach for targeting liposomes to liver cells (Kamps et al., Proc. Nat'l Acad. Sci. USA 94:11681, 1997). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a counter-receptor expressed by the target cell (Harasym et al., Adv. Drug Deliv. Rev. 32:99, 1998). After plasma elimination of free antibody, streptavidin-conjugated liposomes are administered. In another approach, targeting antibodies are directly attached to liposomes (Harasym et al., Adv. Drug Deliv. Rev. 32:99, 1998).

Anti-pG6b neutralizing antibodies and binding partners with pG6b binding activity, or pG6b soluble receptor, can be encapsulated within liposomes using standard techniques of protein microencapsulation (see, e.g., Anderson et al., Infect. Immun. 31:1099, 1981; Anderson et al., Cancer Res. 50:1853, 1990; Cohen et al., Biochim. Biophys. Acta 1063:95, 1991; Alving et al. “Preparation and Use of Liposomes in Immunological Studies,” in Liposome Technology, 2nd Edition, Vol. III, Gregoriadis (ed.), page 317 (CRC Press 1993); Wassef et al., Meth. Enzymol 149:124, 1987). As noted above, therapeutically useful liposomes may contain a variety of components. For example, liposomes may comprise lipid derivatives of poly(ethylene glycol) (Allen et al., Biochim. Biophys. Acta 1150:9, 1993).

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer (Gombotz and Pettit, Bioconjugate Chem. 6:332, 1995; Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161, 1998; Putney and Burke, Nature Biotechnology 16:153, 1998; Putney, Curr. Opin. Chem. Biol. 2:548, 1998). Polyethylene glycol (PEG)-coated nanospheres can also provide carriers for intravenous administration of therapeutic proteins (see, e.g., Gref et al., Pharm. Biotechnol 10:167, 1997).

The present invention also contemplates chemically modified Anti-pG6b antibody or binding partner, for example anti-pG6b antibodies or pG6b soluble receptor, linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

The present invention contemplates compositions of anti-pG6b antibodies, and methods and therapeutic uses comprising an antibody, peptide or polypeptide described herein. Such compositions can further comprise a carrier. The carrier can be a conventional organic or inorganic carrier. Examples of carriers include water, buffer solution, alcohol, propylene glycol, macrogol, sesame oil, corn oil, and the like.

### 11. Production of Transgenic Mice

Nucleic acids which encode pG6b or modified forms thereof can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding a pG6b protein can be used to clone genomic DNA encoding a pG6b protein in accordance with established techniques and the genomic sequences used to generate transgenic animals that contain cells which express the desired DNA. Methods for generating transgenic animals, particularly animals such as mice or rats, have become conventional in the art and are described, for example, in U.S. Pat. Nos. 4,736,866 and 4,870,009.

Alternatively, non-human homologues of pG6b can be used to construct a “knock out” animal which has a defective or altered gene encoding a pG6b protein as a result of homologous recombination between the endogenous gene and an altered genomic DNA encoding pG6b, which is introduced into an embryonic cell of the animal. For example, cDNA encoding a pG6b protein can be used to clone genomic DNA encoding a pG6b protein in accordance with established techniques. A portion of the genomic DNA encoding a pG6b protein can be deleted or replaced with another gene, such as a gene encoding a selectable marker which can be used to monitor integration. Typically, several kilobases of unaltered flanking DNA (both at the 5′ and 3′ ends) are included in the vector. See e.g., Thomas and Capecchi, Cell 51:503, 1987. The vector is introduced into an embryonic stem cell line (e.g., by electroporation) and cells in which the introduced DNA has homologously recombined with the endogenous DNA are selected. See e.g., Li et al., Cell 69:915, 1992. The selected cells are then injected into a blastocyst of an animal (e.g., a mouse or rat) to form aggregation chimeras. See e.g., Bradley, in Teratocarcinomas and Embryonic Stem Cells: A Practical Approach, E. J. Robertson, ed. (IRL, Oxford, 1987), pp. 113-152. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the pG6b protein. It is understood that the models described herein can be varied. For example, “knock-in” models can be formed, or the models can be cell-based rather than animal models.

The invention is further illustrated by the following non-limiting examples.

### Example 1

### B7/mFc2 Expression Constructs

An expression vector, pZMP21 hB7-H1/mFc2, was prepared to express a c-terminally Fc tagged soluble version of B7-H1. A 734 base pair fragment was generated by PCR containing the extracellular domain of B7-H1 and the first two amino acids of mFc (glutamine and proline) with EcoRI and BglII sites coded on the 5′ and 3′ ends, respectively.

This PCR fragment was generated using primers zc48914 and zc48908 by amplification from a human placenta cDNA library. The PCR reaction conditions were as follows: 25 cycles of 94° C. for 1 minute, 60° C. for 1 minute, and 72° C. for 2 minutes; 1 cycle at 72° C. for 10 minutes; followed by a 4° C. soak. A 699 base pair fragment was generated by PCR containing the constant 2 and constant 3 domains of effector function minus BALB-C IgG gamma 2a (mFc2). This PCR fragment was generated using primers zc48911 and ac48915 by amplification from an expression vector containing mFc2 (mTACI/mFc2 construct #998). The PCR reaction conditions were as follows: 25 cycles of 94° C. for 1 minute, 60° C. for 1 minute, and 72° C. for 2 minutes; 1 cycle at 72° C. for 10 minutes; followed by a 4° C. soak. The 734 base pair B7-H1 fragment and the 699 base pair mFc2 fragment were purified by 1% agarose gel electrophoresis and band purification using a QiaQuick gel extraction kit (Qiagen: 28704). ⅕th and 1/25th of the total of the purified bands each for the B7-H1 and the mFc2 fragments, respectively were recombined into pZMP21 that had been linearized by BglII digestion and purified by band purification, as described above, using the yeast strain SF838-9Dalpha. Yeast that were able to grow out of uracil deficient agar plates were lysed and DNA was extracted by ethanol precipitation. 2 μl of the ligation mix was electroporated in 37 μl DH10B electrocompetent E. coli (Gibco 18297-010) according to the manufacturer's directions. The transformed cells were diluted in 400 μl of LB media and plated onto LB plates containing 100 μg/ml ampicillin. Clones were analyzed by restriction digests and positive clones were sent for DNA sequencing to confirm PCR accuracy (correct sequence=˜amaf/cbra.dir/pzmp21-hB7Hlmfc2-4322seq.seq).

The expression vector, pZMP21 hB7-H1/mfc2, described above, was then used to build a series of mFc2 soluble chimeric proteins. PG6b/mFc2 was built by PCRing a 431 base pair fragment using oligos zc 50593 and zc50595 with clonetrack #101697 as template. The resulting PCR product was band purified, as described above, and digested with EcoRI and BglII. The resulting product was again band purified. PZMP21 hB7-H1/mFc2 was also digested with EcoRI and BglII and the 9721 base pair vector backbone plus mFc2 was isolated. 1/50^{th} of the pZMP21 hB7-H1/mFc2 product was ligated to 3/50^{th} of the 431 base pair fragment using T4 DNA ligase. 2 μl of the ligation mix was electroporated in 37 μl DH10B electrocompetent E. coli (Gibco 18297-010) according to the manufacturer's directions. The transformed cells were diluted in 400 μl of LB media and plated onto LB plates containing 100 μg/ml ampicillin. Clones were analyzed by restriction digests and positive clones were sent for DNA sequencing to confirm PCR accuracy. The correct construct was designated as hpG6bZMP21

Three sets of 200 μg of the hpG6bZMP21construct were each digested with 200 units of Pvu I at 37° C. for three hours and then were precipitated with IPA and spun down in a 1.5 mL microfuge tube. The supernatant was decanted off the pellet, and the pellet was washed with 1 mL of 70% ethanol and allowed to incubate for 5 minutes at room temperature. The tube was spun in a microfuge for 10 minutes at 14,000 RPM and the supernatant was decanted off the pellet. The pellet was then resuspended in 750 μl of PF-CHO media in a sterile environment, allowed to incubate at 60° C. for 30 minutes, and was allowed to cool to room temperature. 5E6 APFDXB11 cells were spun down in each of three tubes and were resuspended using the DNA-media solution.

The DNA/cell mixtures were placed in a 0.4 cm gap cuvette and electroporated using the following parameters: 950 μF, high capacitance, and 300 V. The contents of the cuvettes were then removed, pooled, and diluted to 25 mLs with PF-CHO media and placed in a 125 mL shake flask. The flask was placed in an incubator on a shaker at 37° C., 6% CO₂, and shaking at 120 RPM.

The cell line was subjected to nutrient selection followed by step amplification to 200 nM methotrexate (MTX), and then to 500 nM MTX. Expression was confirmed by western blot.

### Example 2

### Human PG6bAvi-HIS TagpZMP21

In the effort to create the tetramer molecules an expression plasmid containing a polynucleotide encoding the extra-cellular domain of human pG6b, the Avi Tag and HIS Tag was constructed. A DNA fragment of the extra-cellular domain of human pG6b is isolated by PCR using the polynucleotide sequence:

ATGGCTGTGTTTCTGCAGCTGCTACCGCTGCTGCTCTCGAGGGCCCAAGGGAACCCTGGG GCTTCTCTGGACGGCCGCCCTGGGGACCGGGTGAATCTCTCCTGCGGAGGAGTCTCTCAT CCCATCCGCTGGGTCTGGGCACCCAGCTTCCCGGCCTGCAAGGGCCTGTCCAAAGGACGC CGACCGATCCTGTGGGCCTCTTCGAGCGGGACCCCCACCGTGCCTCCCCTCCAGCCTTTC GTCGGCCGCCTACGCTCCCTGGACTCTGGTATCCGGCGGCTGGAGCTCCTCTTGAGCGCG GGGGACTCGGGCACTTTTTTCTGCAAGGGCCGCCACGAGGACGAGAGCCGTACAGTGCT TCACGTGCTGGGGGACAGGACCTATTGCAAGGCCCCCGGGCCTACCCATGGGTCC with flanking regions at the 5′ and 3′ ends corresponding to the vector sequence and part of the Avi Tag sequence flanking the human pG6b insertion point using primers zc51120 (CCACAGGTGTCCAGGGAATTCGCAAGATGGCTGTGTTTCTGCAG) and zc51122 (CTCCACCAGATCCCTTGCGGGACCCATGGGTAGGCCC).

The PCR reaction mixture is run on a 2% agarose gel and a band corresponding to the size of the insert is gel-extracted using a QIAquick™ Gel Extraction Kit (Qiagen, Valencia, Calif.). Plasmid pZMP21 is a mammalian expression vector containing an expression cassette having the MPSV promoter, multiple restriction sites for insertion of coding sequences, a stop codon, an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae. It is constructed from pZP9 (deposited at the American Type Culture Collection, 10801 University Boulevard, Manassas, Va. 20110-2209, under Accession No. 98668) with the yeast genetic elements taken from pRS316 (deposited at the American Type Culture Collection, 10801 University Boulevard, Manassas, Va. 20110-2209, under Accession No. 77145), an internal ribosome entry site (IRES) element from poliovirus, and the extracellular domain of CD8 truncated at the C-terminal end of the transmembrane domain. Plasmid pZMP21AviHIS was digested with EcoRI and used for recombination with the PCR insert.

The recombination was performed using the BD In-Fusion™ Dry-Down PCR Cloning kit (BD Biosciences, Palo Alto, Calif.). The mixture of the PCR fragment and the digested vector in 10 μl was added to the lyophilized cloning reagents and incubated at 37° C. for 15 minutes and 50° C. for 15 minutes. The reaction was ready for transformation. 2 μl of recombination reaction was transformed into One Shot TOP10 Chemical Competent Cells (Invitrogen, Carlsbad, Calif.); the transformation was incubated on ice for 10 minutes and heat shocked at 42° C. for 30 seconds. The reaction was incubated on ice for 2 minutes (helping transformed cells to recover). After the 2 minutes incubation, 300 μl of SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was added and the transformation was incubated at 37° C. with shaker for one hour. The whole transformation was plated on one LB AMP plates (LB broth (Lennox), 1.8% Bacto™ Agar (Difco), 100 mg/L Ampicillin).

The colonies were screened by PCR using primers zc51120 and zc51122. The positive colonies were verified by sequencing. The correct construct was designated as hPG6bAviHISpZMP21.

### Example 3

### pG6b Antibodies

Rabbits were injected with pG6b-mouse-Fc2 fusion protein conjugated to BSA. Rabbits with positive serum titers to pG6b were bled and serum collected. Serum was purified by use of a pG6b-Fc2 affinity column.

For monoclonal antibodies, BALB/c mice were immunized with pG6b-mouse-Fc2 fusion protein conjugated to BSA. Mice with positive serum titers to cellular expressed human pG6b were given a prefusion boost of soluble pG6b-Fc fusion protein. Splenocytes were harvested from one high-titer mouse and fused to P3-X63-Ag8/ATCC (mouse) myeloma cells in an optimized PEG-mediated fusion protocol (Rockland Immunochemicals). Following 12 days growth post-fusion, specific antibody-producing hybridoma pools were identified using FMAT (Applied Biosystems) screening. In this assay, a receptor presenting cell line (p815-pG6b) was seeded in 96 well tissue culture plates at 100 μL/well, 5×10⁴ cells/ml (plated the day before the assay run). Serial 10-fold dilutions (in cell culture media) of the sera were prepared beginning with an initial dilution of 1:100 and ranged to 1:100,000. Duplicate samples of each dilution were then transferred to the assay plate, 5 μL/well. The secondary antibody, FMAT Blue Goat anti Mouse IgG, Fc specific, was diluted to 0.26 μg/ml in cell culture media and 50 μL/well was added to each well. The plate was incubated in the dark (wrapped in foil) for 4 hours at room temperature. The plate was read on the FMAT 8200 Cellular Detection System.

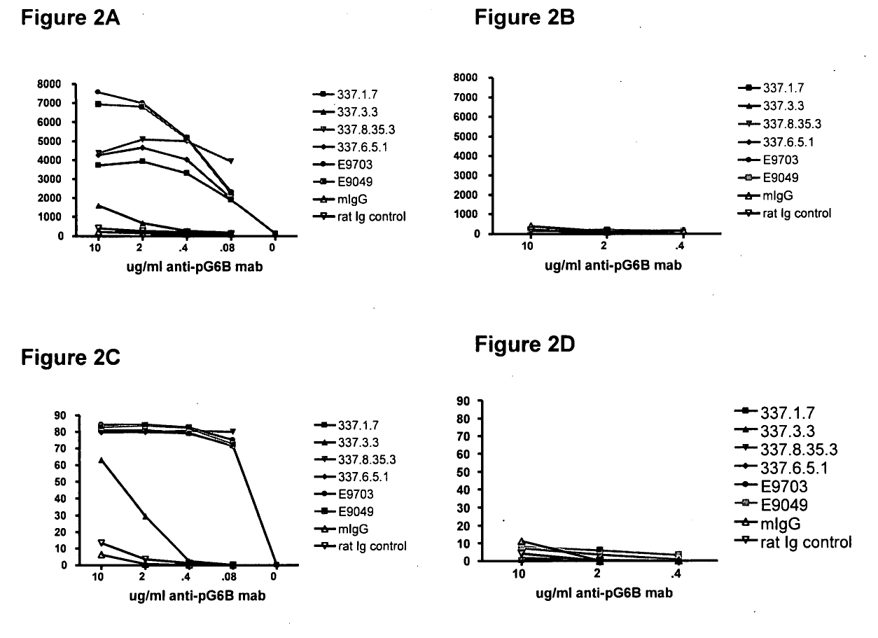

To check for cross-reactivity, the samples were also checked against wild-type p815 cells. Hybridoma pools positive to the specific antibody target only were analyzed further for ability to bind via FACS analysis to p815/pG6b cells but not parental p815 cells as antibody target. (See FIGS. 2A-2D.)

Hybridoma pools yielding a specific positive result in the FMAT assay and positive results in the FACS assay were cloned at least two times by limiting dilution.

The following masterwells and clones were harvested and purified for use in assays: masterwells 337.1, 337.2, 337.3, 337.4, 337.5, 337.6, and 337.7; and clones 337.8, 337.1.7, 337.3.3, 337.6.5, and 337.8.35

### Example 4

### Expression on Human PBMNC

In order to culture the cells, blood from normal in-house donors was separated on a ficol gradient, and the PBMNC interface collected and washed in PBS. The cells were counted and plated in 96 well round bottom plates at 2×10⁵ cells/well in 200 ul culture medium with either LPS at 100 ng/ml or with anti-CD3+anti-CD28 mabs (50 ng/ml and 1 ug/ml respectively). Some cells were reserved for the time 0 timepoint. Cells were collected for staining at times 24, 48 and 72 hours.

At each timepoint, cells in 96 well plates are spun, the media flicked out, and a combination of fluor-conjugated antibodies to surface lineage markers added in 50 ul Facs staining buffer (CD56-A488, CD19-PE, CD45RA-Cychrome, CD45RO-PE-Cy7, CD4-A405, CD8-A700, and CD14-A750). The combination included either mab anti-pG6b (337.8.35) coupled to A647 dye, or a control mab similarly coupled. In some experiments, the binding of mab anti-pG6b was competed with 20 fold (g/g) excess pG6b receptor. Each condition was stained in triplicate wells. Cells were incubated with a stain combo for 30 minutes on ice, then are washed 1.5× with Facs buffer and fixed with 2% paraformaldehyde, 100 ul/well, for 10 minutes, at room temp. Plates were spun, the paraformaldehyde flicked out, and cells resuspended in 200 ul Facs buffer and stored at 4° C. foil-covered until they were read on the LSRII.

The LSRII data was analyzed using FacsDiva software. FSC X SSC dot plots were used to determine a viable cell population gate. Viable cells were then analyzed for anti-pG6b binding using dot plots of anti-pG6b-A647 vs specific lineage markers.

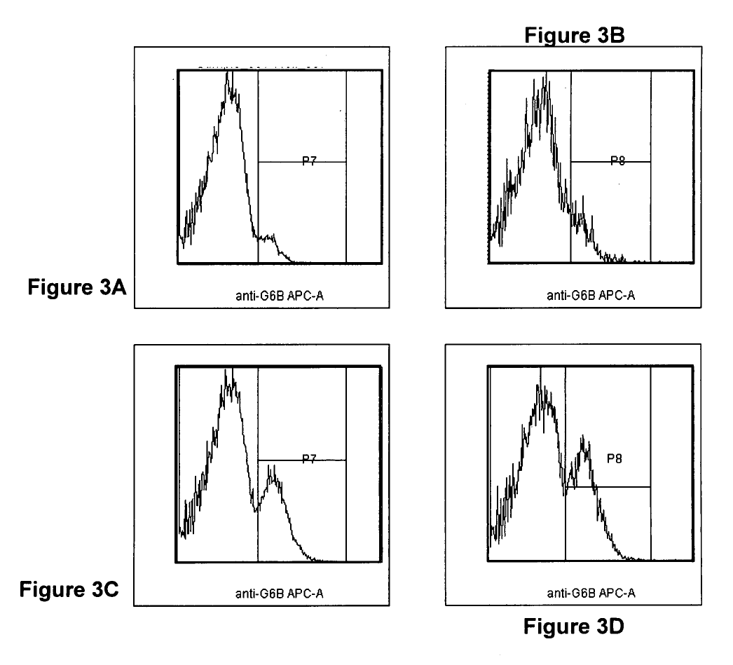

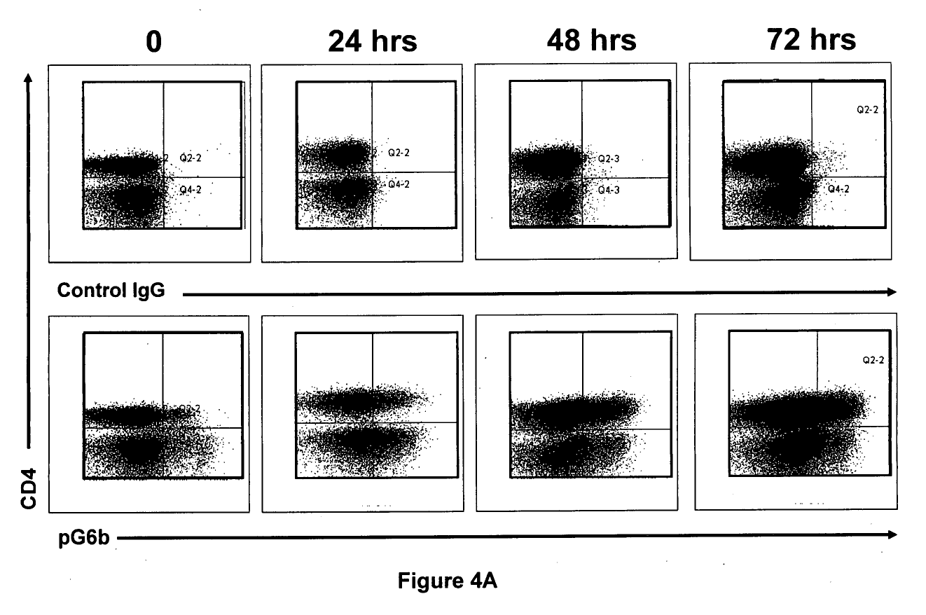

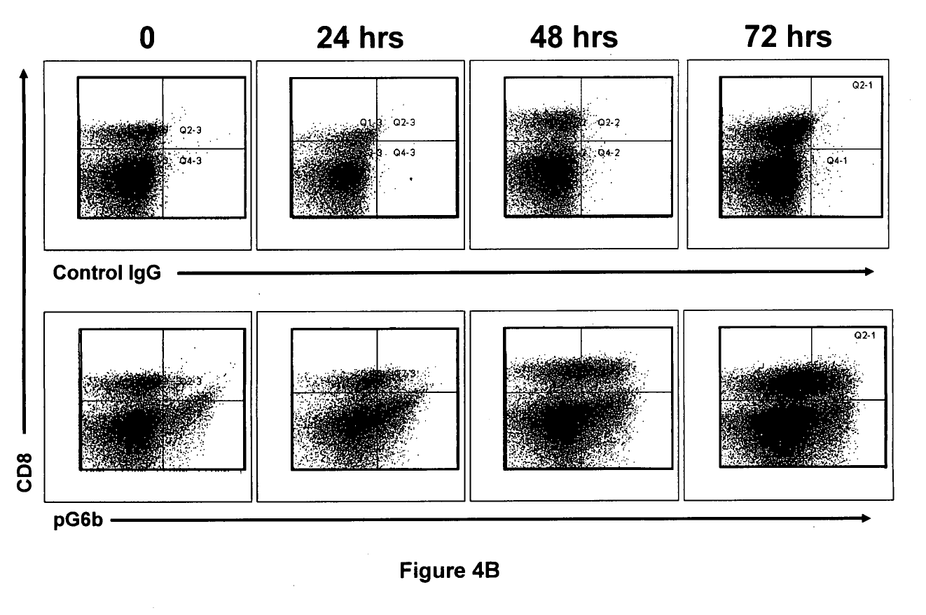

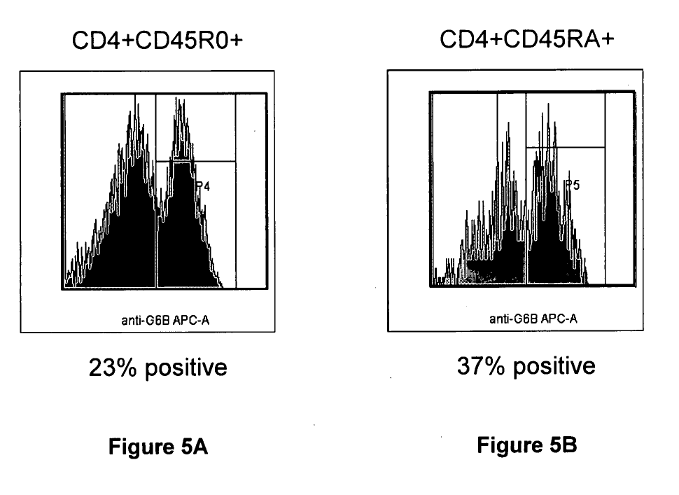

The results indicated that pG6b is expressed on resting CD4⁺ and CD8⁺ cells (see FIGS. 3A-3D) and that expression is upregulated with activation on CD4⁺ and CD8⁺ cells (see FIGS. 4A and 4B). There is no detectable binding on CD 19⁺ and there is no competable binding to CD14⁺ or CD11c cells. Expression of pG6b was higher on naïve T cells relative to memory T cells. (See FIGS. 5A and 5B.)

### Example 5

### T-Cell Proliferation is Modulated by pG6b Antibodies

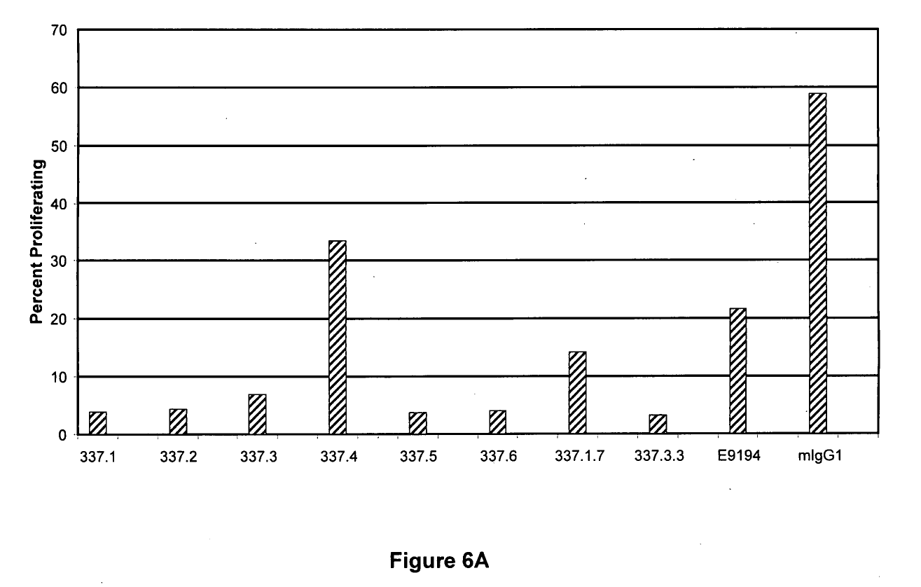

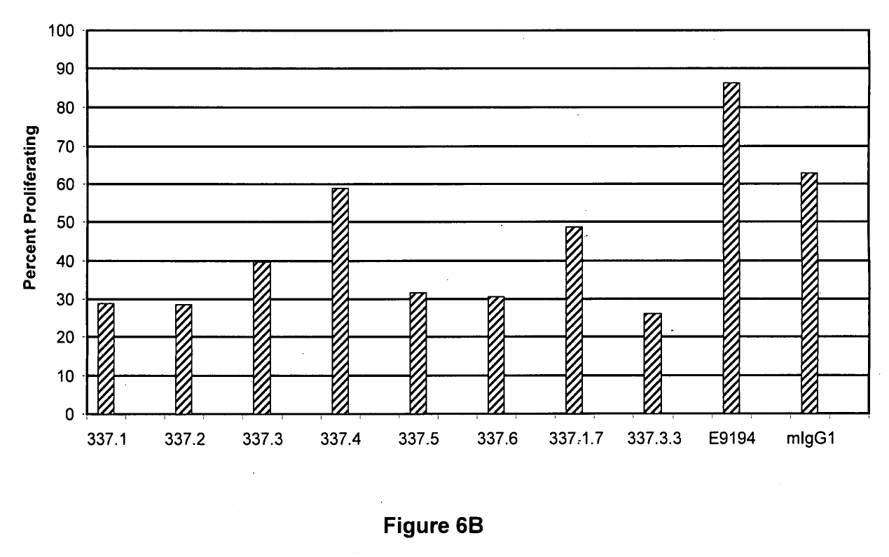

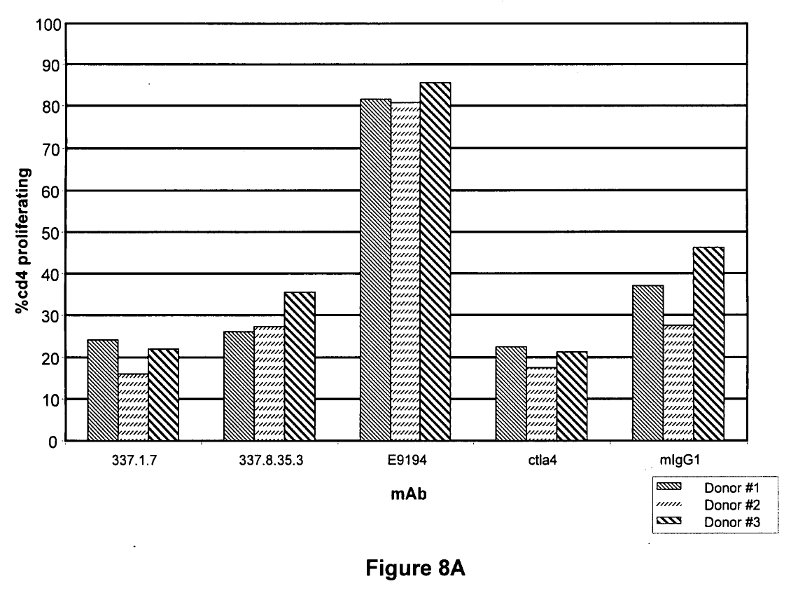

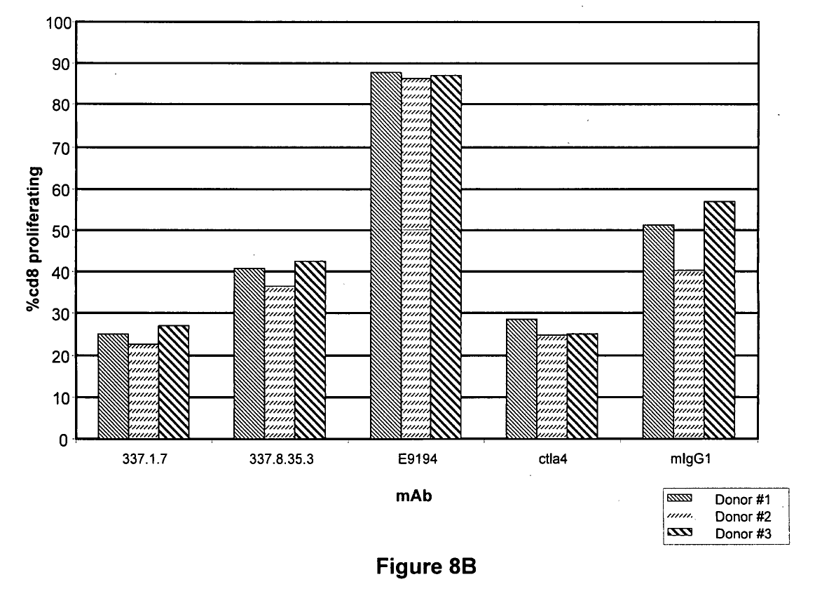

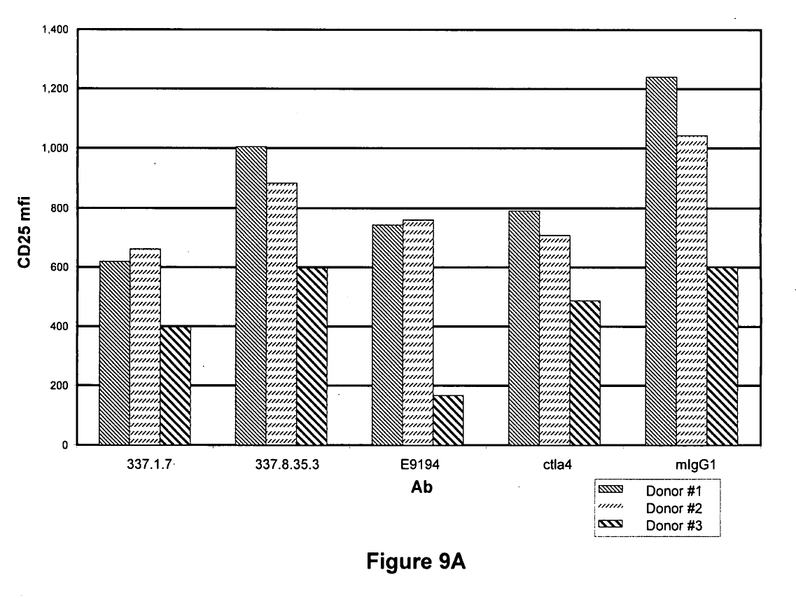

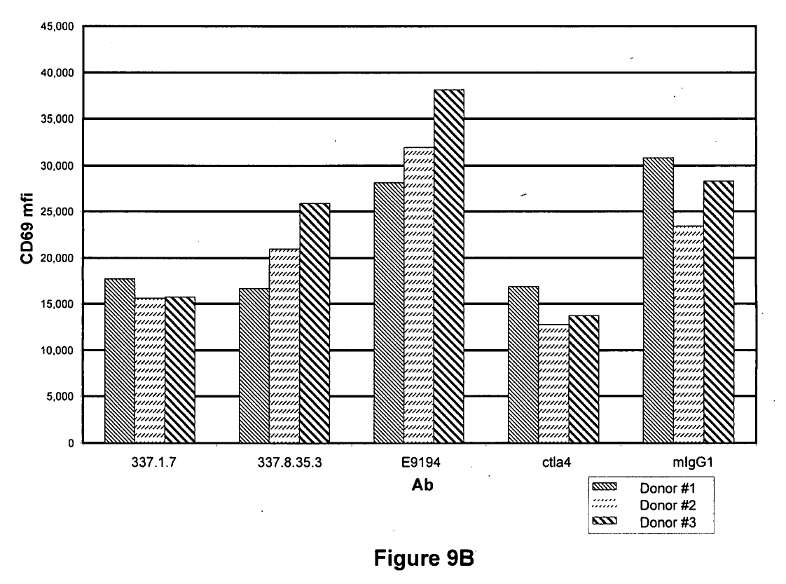

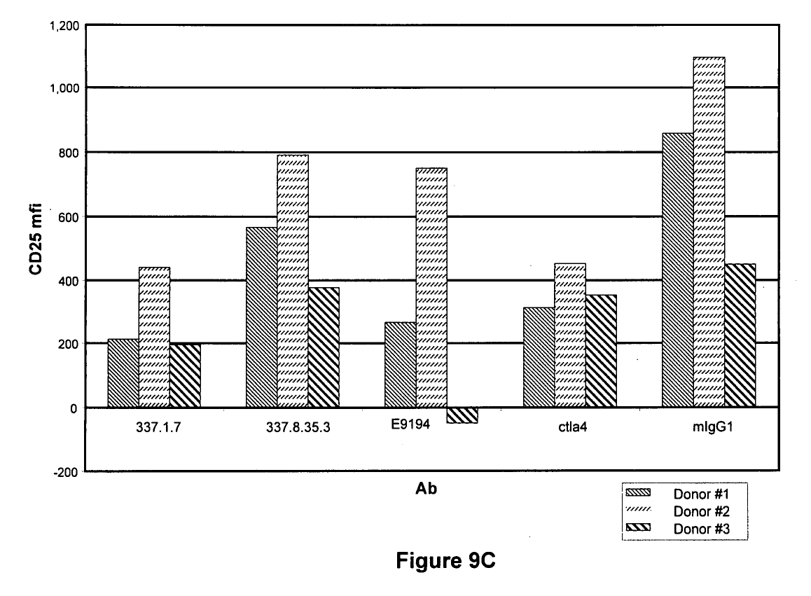

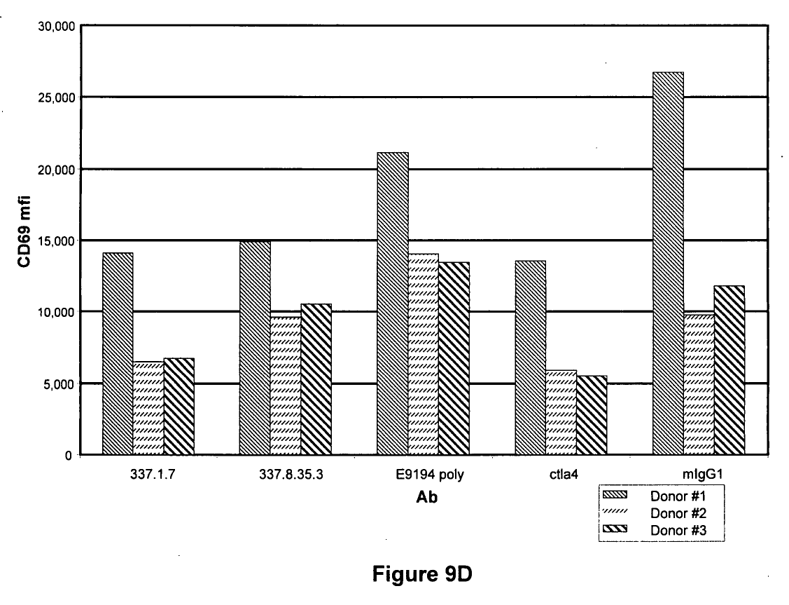

The proliferation of purified CD4⁺ and CD8⁺ T cells from human peripheral blood mononuclear cells (PBMC) was inhibited by antibody to pG6b in vitro. An antibody to CD3 (BD Biosciences 555329) mimicked T cell antigen recognition. Engagement of CD3 and the T cell receptor (TcR) by antibody provided a signal to proliferate in vitro. This signal was enhanced or inhibited by additional signals. Antibodies to pG6b, covalently coupled to tosylactivated 4.5μ beads (Dynal 140.13), inhibited the anti-CD3-induced proliferation of T cells in vitro by 50-90%. (See FIGS. 6A, 6B, 7A, and 7B.) The addition of co-stimulatory anti-CD28 (BD Biosciences 555725) partially overcame the inhibitory effect of anti-pG6b. (See FIGS. 8A and 8B.) Moreover, anti-pG6b inhibited the expression of the early activation markers CD69 and the IL-2 receptor CD25. (See FIGS. 9A-9D.)

Antibodies were coupled to Dynabeads M-450 Tosylactivated, (Dynal Biotech ASA, Oslo, Norway) as follows: 50 μl (2×10⁷ beads) per sample was washed once with 1 ml 0.1M sodium phosphate buffer, pH 7.4-8.0 in a 2.0 ml eppendorf tube. The tube was placed in a magnet for 1 minute and the supernatant was removed. The beads were resuspended in the original volume using the sodium phosphate buffer. 2.5 μg of anti-CD3 (BD Biosciences 555329) and 10 μg of anti-pG6b, mouse IgG (R&D Systems) or anti-CTLA-4 (R&D Systems: clone #48815, catalog number MAB325) were combined with 50 μl washed beads in 2.0 ml eppendorf tubes. A beads only (no antibody) control was included. The tubes were placed on a Clay Adams Nutator mixer (Bectin-Dickinson, Franklin Lakes, N.J.) at room temperature for 48 hours. The tubes were then placed in a magnet for 1 minute and the supernatant was removed. The coated beads were then washed 4 times with 1 ml PBS (without Ca₂⁺ and Mg₂⁺), 0.1% BSA (w/v) and 2 mM EDTA, pH 7.4.

Tosylactivated beads were used as a solid phase platform to present anti-CD3 and anti-pG6b to T cells. Human PBMC from healthy volunteers were collected by Ficoll-Paque (GE Healthcare) density gradient. In certain experiments, CD4 and CD8 were co-purified from PBMC by magnetic bead columns (Miltenyi Biotec). T cells were labeled with CFSE (Invitrogen) to assess proliferation by flow cytometry. 1×10⁵ CFSE-labeled T cells and 1×10⁵ beads were plated per well in RPMI “complete” media (10% FCS, 2 mM L-Glutamine, 1 mM Na-Pyruvate, 0.1 mM NEAA, 0.05 mM β-ME). Cultures were maintained for 1 day to assess early activation markers or 3 days to assess proliferation in humidified incubators at 5% CO₂. Proliferation of CD4⁺ and CD8⁺ cells was measured on an LSRII (Becton Dickinson) by gating on individual cell populations and measurement of CFSE dilution. In specific experiments, anti-CD28 (BD Biosciences 555725) was added in solution at a final concentration of 1 μg/ml.

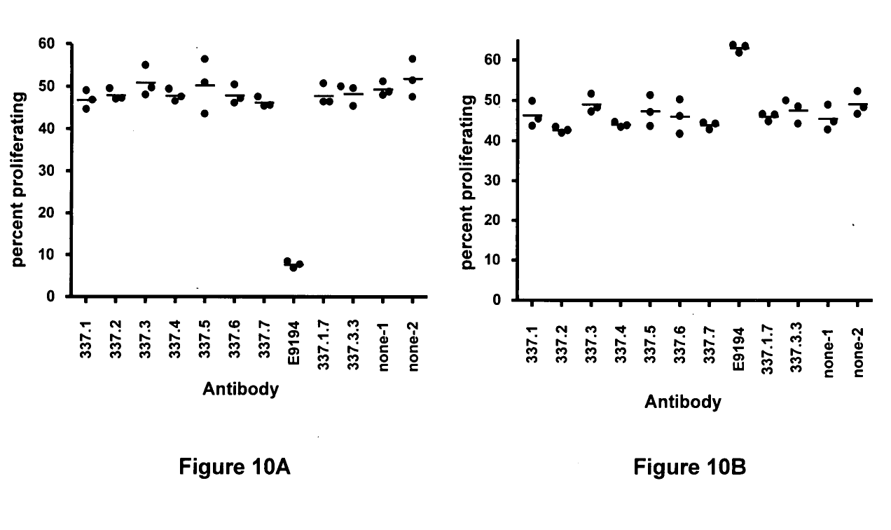

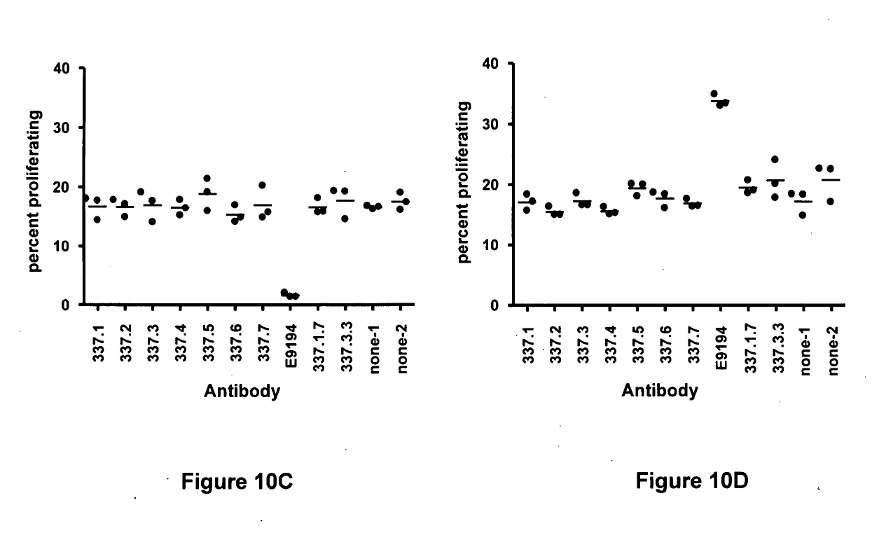

In separate experiments using soluble antibodies, anti-pG6b polyclonal sera inhibited the response of human CD4⁺ and CD8⁺ cells stimulated with soluble anti-CD3. (See FIGS. 10A and 11B.) Human PBMC were prepared from healthy volunteers and collected by Ficoll-Paque (GE Healthcare) density gradient. CD4⁺ and CD8⁺ were co-purified from PBMC by magnetic bead columns (Miltenyi Biotec). T cells were labeled with CFSE (Invitrogen) to assess proliferation by flow cytometry. 1×10⁵ CFSE-labeled T cells and 100 ng/ml anti-CD3 were plated per well. Where indicated, anti-pG6b, mouse IgG or anti-CTLA-4 were added at 1 μg/ml final concentration. Cultures were maintained for 3 days to assess proliferation in humidified incubators at 5% CO₂. Proliferation of CD4s and CD8s was measured on an LSRII (Becton Dickinson) by gating on individual cell populations and measurement of CFSE dilution. In specific experiments, anti-CD28 (R&D Systems) was added in solution at a final concentration of 1 μg/ml. Inclusion of anti-CD28 to the cultures containing polyclonal anti-pG6b resulted in an increased proliferative response above that observed with anti-CD3 alone. (See FIGS. 10B and 11B).

Both the inhibitory and stimulatory effects of anti-pG6b were not donor specific in that 3/3 donors tested all displayed similar activity.

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

What is claimed is:

1. An isolated anti-pG6b monoclonal antibody that competes for binding to the extracellular domain of human pG6b (SEQ ID NO:3) with an antibody selected from:
(a) the antibody produced by the hybridoma of clone designation number 337.1.4 (ATCC Patent Deposit Designation PTA-8730);
(b) the antibody produced by the hybridoma of clone designation number 337.3.3 (ATCC Patent Deposit Designation PTA-8731);
(c) the antibody produced by the hybridoma of clone designation number 337.6.5.1 (ATCC Patent Deposit Designation PTA-8728); and
(d) the antibody produced by the hybridoma of clone designation number 337.8.35.3 (ATCC Patent Deposit Designation PTA-8729).

2. The anti-pG6b antibody of claim 1, wherein the anti-pG6b antibody comprises
a heavy chain variable region comprising CDRs H1, H2, and H3 of the antibody selected from (a), (b), (c), and (d); and
a light chain variable region comprising CDRs L1, L2, and L3 of the antibody selected from (a), (b), (c), and (d).

3. The anti-pG6b antibody of claim 2, wherein the anti-pG6b antibody comprises
the heavy chain variable region of the antibody selected from (a), (b), (c), and (d); and
the light chain variable region of the antibody selected from (a), (b), (c), and (d).

4. The anti-pG6b antibody of claim 7, wherein the anti-pG6b antibody is the antibody selected from (a), (b), (c), and (d).

5. The anti-pG6b antibody of claim 2, wherein the anti-pG6b antibody is a humanized antibody.

6. The anti-pG6b antibody of claim 1, wherein the anti-pG6b antibody is a single-chain antibody.

7. A method for enhancing a T cell-mediated host immune response to a target antigen in a subject, the method comprising:
administering to the subject a soluble anti-pG6b antibody, wherein said antibody is an isolated anti-pG6b antibody as in claim 1, and wherein said administration is effective to enhance a T cell-mediated host immune response to the target antigen in the subject.

8. The method of claim 7, wherein said antibody is an isolated anti-pG6b antibody as in any one of claims 2 to 6.

9. A method for treating cancer in a subject, the method comprising:
administering to the subject a soluble anti-pG6b antibody, wherein said antibody is an isolated anti-pG6b antibody as in claim 1, and wherein said administration is effective to increase a host immune response against tumor cells in the subject.

10. The method of claim 9, wherein said antibody is an isolated anti-pG6b antibody as in any one of claims 2 to 6.

11. A hybridoma selected from the group consisting of:
(a) the hybridoma of clone designation number 337.1.4 (ATCC Patent Deposit Designation PTA-8730);
(b) the hybridoma of clone designation number 337.3.3 (ATCC Patent Deposit Designation PTA-8731);
(c) the hybridoma of clone designation number 337.6.5.1 (ATCC Patent Deposit Designation PTA-8728); and
(d) the hybridoma of clone designation number 337.8.35.3 (ATCC Patent Deposit Designation PTA-8729).


SEQUENCE LISTING (7 sequences)

SEQ ID NO: 1 - DNA, 726 bp, Homo sapiens
  CDS (1)...(726)
atggctgtgtttctgcagctgctaccgctgctgctctcgagggcccaagggaaccctggggcttctctggacggccgccctggggaccgggtgaatctctcctgcggaggagtctctcatcccatccgctgggtctgggcacccagcttcccggcctgcaagggcctgtccaaaggacgccgaccgatcctgtgggcctcttcgagcgggacccccaccgtgcctcccctccagcctttcgtcggccgcctacgctccctggactctggtatccggcggctggagctcctcttgagcgcgggggactcgggcacttttttctgcaagggccgccacgaggacgagagccgtacagtgcttcacgtgctgggggacaggacctattgcaaggcccccgggcctacccatgggtccgtgtatccccagctcctgatcccgctgctgggcgctgggttggtgctcggactgggagctttgggcctggtctggtggctgcacaggcgcctgcccccgcaaccgattcgaccactccctagatttgctccacttgtgaaaaccgagccccagaggccagtaaaggaggaagagcccaagattccaggggacctggaccaggaaccgagcctgctctatgcggatctggaccatctagccctcagcaggccccgccggctgtccacagcggaccctgctgatgcctccaccatctatgcagttgtagtttga

SEQ ID NO: 2 - protein, 241 aa, Homo sapiens
MAVFLQLLPLLLSRAQGNPGASLDGRPGDRVNLSCGGVSHPIRWVWAPSFPACKGLSKGRRPILWASSSGTPTVPPLQPFVGRLRSLDSGIRRLELLLSAGDSGTFFCKGRHEDESRTVLHVLGDRTYCKAPGPTHGSVYPQLLIPLLGAGLVLGLGALGLVWWLHRRLPPQPIRPLPRFAPLVKTEPQRPVKEEEPKIPGDLDQEPSLLYADLDHLALSRPRRLSTADPADASTIYAVVV

SEQ ID NO: 3 - protein, 142 aa, Homo sapiens
MAVFLQLLPLLLSRAQGNPGASLDGRPGDRVNLSCGGVSHPIRWVWAPSFPACKGLSKGRRPILWASSSGTPTVPPLQPFVGRLRSLDSGIRRLELLLSAGDSGTFFCKGRHEDESRTVLHVLGDRTYCKAPGPTHGSVYPQ

SEQ ID NO: 4 - DNA, 699 bp, Mus musculus
  CDS (1)...(699)
atggccttggtcctgccgctgctgcctttgttgctctcaaaggtccaggggaatcccgaggtttctttggagggcagccctggggaccgggtgaatctctcctgcataggggtctccgaccccacccgctgggcttgggcgcctagtttcccagcatgcaagggcttgtctaaagggcgccgtccgatcttgtgggcctcgacgagagggaccccaactgtgctccagcatttctctggccgcctgcgttccctggacaatggtatcaagcggctagagctgctgctgagcgccggggattctggaacctttttctgcaaaggacgccacgagaatgagagtcgcacagtgcttcaagtgttaggggacaaggcaggttgccggcctgcaggatctacccacgggtacgagtatcccaaggtcctgattccgctcctgggcgttgggcttgtgctgggactgggagtcgcgggcgtggtctggcggcggcgcagttctgtccctccctcccacatagctccagtcataaatgctgagccacagaggcctttagaacaggagtccaagatctcaggccacctggaccaggagccgagcctgcactacgctgatctggaccactctgtcctcgggaggcaccgccggatgtccacagtggtttctggtgatgcctccactgtctatgcggttgtagtataa

SEQ ID NO: 5 - protein, 232 aa, Mus musculus
MALVLPLLPLLLSKVQGNPEVSLEGSPGDRVNLSCIGVSDPTRWAWAPSFPACKGLSKGRRPILWASTRGTPTVLQHFSGRLRSLDNGIKRLELLLSAGDSGTFFCKGRHENESRTVLQVLGDKAGCRPAGSTHGYEYPKVLIPLLGVGLVLGLGVAGVVWRRRSSVPPSHIAPVINAEPQRPLEQESKISGHLDQEPSLHYADLDHSVLGRHRRMSTVVSGDASTVYAVVV

SEQ ID NO: 6 - protein, 140 aa, Mus musculus
MALVLPLLPLLLSKVQGNPEVSLEGSPGDRVNLSCIGVSDPTRWAWAPSFPACKGLSKGRRPILWASTRGTPTVLQHFSGRLRSLDNGIKRLELLLSAGDSGTFFCKGRHENESRTVLQVLGDKAGCRPAGSTHGYEYPK

SEQ ID NO: 7 - protein, 138 aa, Homo sapiens
MAVFLQLLPLLLSRAQGNPGASLDGRPGDRVNLSCGGVSHPIRWVWAPSFPACKGLSKGRRPILWASSSGTPTVPPLQPFVGRLRSLDSGIRRLELLLSAGDSGTFFCKGRHEDESRTVLHVLGDRTYCKAPGPTHGS